(19) United States
(12) Patent Application Publication
(10) Pub. No.: US 2006/0040894 A1
(43) Pub. Date: Feb. 23, 2006

(54) Compositions and methods using hyaluronic acid

(75) Inventors: William L. Hunter, Vancouver (CA); David M. Gravett, West Vancouver (CA); Philip M. Toleikis, Vancouver (CA); John K. Jackson, Vancouver (CA)

Correspondence Address: DARBY & DARBY P.C., P. O. BOX 5257, NEW YORK, NY, 10150-5257, US

(73) Assignees: Angiotech International AG, Zug (CH); University of British Columbia, Vancouver (CA)

(21) Appl. No.: 11/205,287

(22) Filed: Aug. 15, 2005

Related U.S. Application Data
(60) Provisional application No. 60/601,214, filed on Aug. 13, 2004.
(60) Provisional application No. 60/601,218, filed on Aug. 13, 2004.

(51) Int. Cl. A61K 31/728; A61K 31/28

(52) U.S. Cl. 514/54; 514/495

(57) ABSTRACT

Compositions and devices including hyaluronic acid and a compound that inhibits degradation of hyaluronic acid, and methods of making and using same.

### CROSS-REFERENCE TO RELATED APPLICATION

This application claims the benefit of U.S. Provisional Patent Application Nos. 60/601,214 and 60/601,218, both filed on Aug. 13, 2004, which provisional applications are incorporated herein by reference in their entirety.

### BACKGROUND

1. Technical Field

The present invention relates generally to pharmaceutical compositions, devices and methods, and more specifically, to compositions, devices and methods related to enhancing the duration and activity of implanted hyaluronic acid materials.

2. Description of Related Art

Hyaluronic acid (HA) is a ubiquitous material found naturally in many body tissues including synovial joint fluid, vitreous humor in the eye, cartilage, blood vessels, extracellular matrix, skin and the umbilical cord. Retention of water is one of the most important biological functions of hyaluronic acid, second only to providing nutrients and removing waste from cells that do not have a direct blood supply, such as cartilage cells. The ability of HA to bind water gives structure, to tissue, lubricates and cushions moveable parts of the body, such as joints (e.g., knee) and muscles, and contributes to the skin's volume.

The ability of hyaluronic acid to act as a lubricant and to provide structural support has led to its use in a wide variety of medical applications, including, for example, ophthalmology, soft tissue augmentation (e.g., HA implants for use in plastic and reconstructive surgery), wound care, viscosupplementation of joints (e.g., intra-articular injections), bone regeneration, adhesion prevention, drug delivery, cell preservation, surface coatings, and moisturizing agents. A particular advantage of HA over other types of biomaterials (e.g., collagen) is that since HA is part of the natural extracellular matrix, the body does not produce an immunogenic (allergic) response to HA-based implants.

Hyaluronic acid, however, has a relatively limited lifetime when implanted into the body. The durability of the implant in vivo can be compromised by the activity of various degradative enzymes, such as hyaluronidase. Hyaluronidase refers, in general, to hydrolytic enzymes, such as hyaluronate lyase and hyaluronoglucuronidase, which can catalyze the cleavage of internal glycosidic bonds of certain acid mucopolysaccharides found in animal connective tissues (e.g., sodium hyaluronic acid and sodium chondroitin sulphate A and C). For example, hyaluronoglucosamimidase catalyzes the hydrolysis of random β-1,4 linkages between N-acetylglucosamine and D-glucuronic acid residues in hyaluronic acid. It also hydrolyzes chondroitin, chondroitin 4- and 6-sulphates, and dermatan sulphate. Hyaluronoglucuronidase catalyzes the hydrolysis of β-1,3 linkages between glucuronic acid and N-acetylglucosamine residues in HA. Hyaluronate lyase catalyzes the fragmentation of HA via an elimination reaction in which the bond from N-acetylflucosamine to glucuronate is broken and a double bond introduced. As a result of enzymatic breakdown of HA in the body after implantation, the functional activity of HA in the body after administration is limited. Because of this, medical procedures utilizing HA as an implant (especially, for example, cosmetic enhancement or tissue bulking agents) often require repeat administration on a regular basis. For example, HA-based dermal implant and viscosupplementation treatment must be repeated every 6 to 9 months.

The present invention addresses shortcomings associated with hyaluronic acid and the use thereof in medical applications, and provides other related advantages.

### BRIEF SUMMARY

Briefly stated, the present invention provides compositions, devices, and methods for prolonging the activity of hyaluronic acid-based implants. Hyaluronic acid-based implants are used to provide structure, support, and lubrication in a variety of medical procedures including, for example, dermal injections for cosmetic purposes (to reduce wrinkles, scars, contour defects), intra-articular injections to relieve joint pain, vascular “plugs” to produce hemostasis following vascular puncture procedures, and “bulking agents” to treat urinary incontinence, fecal incontinence and gastro-esophageal reflux.

In one aspect, the present invention provides compositions that combine hyaluronic acid and an inhibitory compound (i.e., inhibitor), where the inhibitory compound can inhibit the activity of hyaluronidase. HA compositions containing such compounds are not broken down by the body as quickly and can be used to produce a hyaluronic acid-based implant with enhanced durability and longevity in vivo.

A variety of inhibitory compounds are described within the context of the present invention. In separate embodiments, each of the inhibitory compounds described herein is capable of inhibiting degradation of hyaluronic acid. In certain embodiments, the inhibitory compounds inhibit the enzyme-induced degradation of hyaluronic acid by a hyaluronidase. In one aspect, the present invention provides a composition comprising hyaluronic acid and a gold compound, wherein the gold compound (e.g., organo-gold compound) inhibits degradation of hyaluronic acid. The composition may further comprise a polymer. In one aspect, the gold compound is aurothiomalate or sodium aurothiomalate. In another aspect, the gold compound is auranofin. In another aspect, the gold compound is gold sodium thiosulphate. In another aspect, the present invention provides a composition comprising hyaluronic acid and indomethacin or an analogue or derivative thereof, wherein the indomethacin inhibits degradation of hyaluronic acid. In another aspect, the present invention provides a composition comprising hyaluronic acid and a sulphate-containing polysaccharide, wherein the sulphate-containing polysaccharide inhibits degradation of hyaluronic acid. The composition may further comprise a polymer. The sulphate-containing polysaccharide may be, e.g., a fucan such as fucoidan or an analogue or derivative thereof; dextran sulphate or an analogue or derivative thereof; or heparin or an analogue or derivative thereof. In another aspect, the present invention provides a composition comprising hyaluronic acid and a polymer, wherein the polymer inhibits degradation of hyaluronic acid. In one aspect, the polymer is a diblock copolymer. In one aspect, the polymer comprises lactic acid residues having the structure (—O—CH(CH₃)—CO—). In another aspect, the polymer comprises ethylene oxide residues having the structure (—OCH₂CH₂—). In another aspect, polymer comprises poly(lactic acid)-co-poly(ethylene glycol) (PLA-PEG). In another aspect, the polymer comprise poly(L-lactic acid)-co-methoxypoly(ethylene glycol) (MePEG-PLLA) (60:40). In another aspect, the polymer comprises poly(lactic-co-glycolic acid)-co-poly(ethylene glycol) (PLGA-PEG). In another aspect, the polymer comprises poly(caprolactone)-co-poly(ethylene glycol) (PCL-PEG). In another aspect, the polymer is a sorbitan ester or a copolymer of ethylene oxide and propylene oxide polymers. The polymer may be a blend of polymers. In one aspect, the polymer is a blend of poly(lactic acid)-co-poly(ethylene glycol) (PLA-PEG) and poly(L-lactic acid)-co-methoxypoly(ethylene glycol) (MePEG-PLLA). In another aspect, the HI is an octylphenol ethoxylate. In yet another aspect, the present invention provides compositions that combine hyaluronic acid and co-solvent type molecules, where these agents inhibit the activity of hyaluronidase and the in vivo degradation of HA. In one aspect, the present invention provides a composition comprising hyaluronic acid and a member selected from polyethylene glycol, propylene glycol, or carboxymethylcellulose (CMC), wherein the member inhibits degradation of hyaluronic acid. In still another aspect, a composition is provided comprising hyaluronic acid and an HI, wherein the HI is Vitamin C, aescin, tranilast, traxanox, hederageenin, guanidine hydrochloride, L-arginine, norlignane, urolithin B, liquirtigenin, baicalein, isoliquiritigenin, disodium cromoglycate (DSCG), chrysin-7-sulphate, sodium flavonone-7-sulphate, sodium-5-hydroxyflavone-7-sulphate, 1-(2-hydroxy-4,6-dimethoxyphenyl)-3-(4-methoxyphenyl)propenone, 1-(2-hydroxy-4,6-dimethoxyphenyl)-3-(4-chlorophenyl)propenone, 7-fluoro-4′-hydroxyflavone-4′-chloro-4,6-dimethoxychalcone, luteolin, morin, myricetin, phenylbutazone, oxypnebutanone, fenoprofen, myocrisin, phosphorylated hesperidin, echinacea, rosmaric acid, sulfonated beta-(1,4)-galacto-oligosaccharides (n=2-6) with degrees of sulfonation from 0.2 to 1; flavanoids such as condensed tannin, tannic acid, kaempferol, quercetin, apeginin; and sulfonated compounds such as sulfonated neomycin, sulfonated planetose, sulphated hydrochinone diglalctoside, or sulphated 2-hydroxy phenyl monolactobioside; and silibin, phloretin, taxifolin, diadzein (4′,7-dihydroxyisoflavone), tectorigenin (4′,7-dihydroxy-6-methoxyisoflavone, chrysin-7-sulphate, 4′-chloro-4,6-dimethoxychalcone, diphenylacrylic acid, diphenylpropionic acid, 3-(4-trifluoromethyl-phenyl)-3-phenylpropionic acid, 3-(4-trifluoromethyl-phenyl)-3-phenylpropionic acid, or indole-2-carboxylic acid; and the composition optionally comprises a polymer. Any of the described compositions may further include a gold compound, wherein the gold compound (e.g., an organo-gold compound or aurothiomalate or an analogue or derivative thereof) inhibits degradation of hyaluronic acid. Compounds that inhibit the degradation of hyaluronic acid by hyaluronidase may be identified using the Hyaluronic Acid Viscometry Assay provided in Example 1 or the GPC Molecular Weight Assay provided in Example 22. In one aspect, a composition is provided that comprises hyaluronic acid and a compound selected from aurothiomalate, indomethacin, fucoidan, dextran sulphate, heparin, polyethylene glycol, propylene glycol, carboxymethylcellulose (CMC), or analogues and derivatives thereof, wherein the viscosity of the composition is 50% or greater of the viscosity of an hyaluronic acid control, wherein the viscosities are measured using the Hyaluronic Acid Viscometry Assay. In another aspect, a composition is provided that comprises hyaluronic acid and a compound selected from octylphenol ethoxylate, sorbitan esters, or copolymers of ethylene oxide and propylene oxide polymers, wherein the viscosity of the composition is 50% or greater of the viscosity of an hyaluronic acid control, wherein the viscosities are measured using the Hyaluronic Acid Viscometry Assay. In another aspect, a composition is provided that comprises hyaluronic acid and a polymer selected from polymers comprising lactic acid residues having the structure (—O—CH(CH₃)—CO—), polymers comprising ethylene oxide residues having the structure (—OCH₂CH₂—), poly(lactic acid)-co-poly(ethylene glycol) (PLA-PEG), poly(L-lactic acid)-co-methoxypoly(ethylene glycol) (MePEG-PLLA) (60:40), poly(lactic-co-glycolic acid)-co-poly(ethylene glycol) (PLGA-PEG), poly(caprolactone)-co-poly(ethylene glycol) (PCL-PEG), or blends thereof, wherein the viscosity of the composition is 50% or greater of the viscosity of an hyaluronic acid control, wherein the viscosities are measured using the Hyaluronic Acid Viscometry Assay. In yet another aspect, a composition is provided that comprises hyaluronic acid and an HI selected from Vitamin C, aescin, tranilast, traxanox, hederageenin, guanidine hydrochloride, L-arginine, norlignane, urolithin B, liquirtigenin, baicalein, isoliquiritigenin, disodium cromoglycate (DSCG), chrysin-7-sulphate, sodium flavonone-7-sulphate, sodium-5-hydroxyflavone-7-sulphate, 1-(2-hydroxy-4,6-dimethoxyphenyl)-3-(4-methoxyphenyl)propenone, 1-(2-hydroxy-4,6-dimethoxyphenyl)-3-(4-chlorophenyl)propenone, 7-fluoro-4′-hydroxyflavone-4′-chloro-4,6-dimethoxychalcone, luteolin, morin, myricetin, phenylbutazone, oxypnebutanone, fenoprofen, myocrisin, phosphorylated hesperidin, echinacea, rosmaric acid, sulfonated beta-(1,4)-galacto-oligosaccharides (n=2-6) with degrees of sulfonation from 0.2 to 1; flavanoids such as condensed tannin, tannic acid, kaempferol, quercetin, apeginin; and sulfonated compounds such as sulfonated neomycin, sulfonated planetose, sulphated hydrochinone diglalctoside, or sulphated 2-hydroxy phenyl monolactobioside; and silibin, phloretin, taxifolin, diadzein (4′,7-dihydroxyisoflavone), tectorigenin (4′,7-dihydroxy-6-methoxyisoflavone, chrysin-7-sulphate, 4′-chloro-4,6-dimethoxychalcone, diphenylacrylic acid, diphenylpropionic acid, 3-(4-trifluoromethyl-phenyl)-3-phenylpropionic acid, 3-(4-trifluoromethyl-phenyl)-3-phenylpropionic acid, or indole-2-carboxylic acid, wherein the viscosity of the composition is 50% or greater of the viscosity of an hyaluronic acid control, wherein the viscosities are measured using the Hyaluronic Acid Viscometry Assay. In yet another aspect, a composition is provided that comprises hyaluronic acid and a compound selected from the group consisting of: heparin (sodium salt), sodium aurothiomalate, carboxymethylcellulose, dextran sulphate, fucoidan, and analogues and derivatives thereof, wherein the molecular weight of the hyaluronic acid is more than about 10%, or more than about 25%, or more than about 50%, or more than about 75%, or more than about 90% of the molecular weight of an hyaluronic acid control, wherein the molecular weights are measured using the GPC Molecular Weight Assay.

In certain aspects, the composition may include two or more HI's. In yet other aspects, the composition includes one or more HI's, wherein one or more of the HI's have an additional therapeutic effect. For example, the HI may also reduce inflammation of tissue at the treatment site (e.g., chrisotherapeutic compounds), may have anticoagulant effects, or may have antiproliferative effects

In still other aspects, the present invention provides compositions composed of a hyaluronidase inhibitor combined with a drug-delivery vehicle (carrier) to provide a sustained release of the agent at the site of HA implantation. In one aspect, the carrier is a polymer. The polymer may be biodegradable or non-biodegradable. In one aspect, the polymer comprises a carbohydrate such as starch, cellulose, and dextran. In another aspect, the polymer comprises a protein such as collagen, gelatin, fibrinogen, and albumin. In another aspect, the polymer comprises a polyester (e.g., poly (D,L lactide), poly (D,L-lactide-co-glycolide), or poly (glycolide)). In another aspect, the polymer comprises poly(ε-caprolactone), poly (hydroxybutyrate), poly (alkylcarbonate), a poly(anhydride), or a poly (orthoester). In another aspect, the polymer comprises an ethylene vinyl acetate copolymer (EVA), silicone rubber, a polyurethane, or an acrylic polymer or copolymer. In one aspect, the polymeric carrier comprises poly(ethylene glycol). In another aspect, the polymeric carrier comprises a 4-armed thiol PEG and a 4-armed NHS PEG and may, optionally, further comprise collagen or a collagen derivative, such as methylated collagen.

In another aspect, compositions are provided that include hyaluronic acid and an HI (e.g., heparin (sodium salt), sodium aurothimalate, carboxy methyl cellulose, dextran sulphate, fucoridan, and analogues and deviations thereof), wherein the HI is contained in a microparticle. The microparticles may be dispersed or contained in a liquid, semi-solid, or solid HA implant to facilitate sustained release of the HI from the composition. In certain embodiments, the HI-loaded microparticles are contained within an HA film or mesh). In other embodiments, the HI-loaded microparticles are dispersed within a liquid or semi-solid form of HA. In certain embodiments, the HI-loaded microparticles are dispersed or incorporated homogeneously within the HA implant.

In yet another aspect, the composition may further comprise a ceramic such as β-tricalcium phosphate, hydroxyapatite, calcium carbonate, calcium sulphate, calcium phosphate, bone, and demineralized bone. In one aspect, the composition may further comprise a bone morphogenic protein (e.g., BMP-2, BMP-3, BMP-4, BMP-5, BMP-6, or BMP-7) a growth factor (e.g., fibroblast growth factor (FGF), transforming growth factor (TGF), or platelet-derived growth factor (PDGF)).

Any of the compositions described herein may further include an anesthetic (e.g., prilocaine, lidocaine, or benzocaine) and/or may be provided in a sterile form.

In other aspects, the present invention provides methods wherein the HA-hyaluronidase inhibitor compositions described herein may be utilized for a variety of clinical indications, including for example: as a dermal implant for cosmetic applications; for viscosupplementation in joints; as a medical device to augment bone growth; as an implant in spinal fusion surgery; as a surgical sling, mesh, or patch; as an implant for the treatment of periodontal disease (e.g., as a dental implant); as a skin graft (e.g., for the development of artificial skin); as a corneal shield; as a tissue bulking agent for the treatment of urinary incontinence, fecal incontinence, or gastro-esophageal reflux; as a surgical adhesion barrier; or as a glaucoma drainage device. In one aspect, the present invention provides a method for augmenting bone or replacing lost bone, comprising, delivering to a patient in need thereof at a desired location a composition as described herein. In another aspect, the present invention provides a method for reducing pain associated with post-surgical scarring, comprising infiltrating an area surrounding a nerve during a surgical procedure with a composition as described herein. In another aspect, the present invention provides a method for preventing surgical adhesions, comprising delivering to a patient in need thereof at a desired location a composition as described herein. In another aspect, the present invention provides a method for the repair or augmentation of skin or tissue, comprising injecting into the skin or tissue of a patient in need thereof a composition as described herein. The injection may be, e.g., into the lips or into the skin on the face. In another aspect, the present invention provides a method for maintaining volume in eye fluid during ocular surgery, comprising delivering to the inside of an eye during an ocular surgery a composition as described herein. The ocular surgery may be, for example, cataract extraction surgery, intraocular lens implantation, retinal reattachment, phacoemulsification surgery, corneal transplantation or glaucoma filtering surgery. In another aspect, the present invention provides a method for reducing pain associated with osteoarthritis, comprising injecting into a joint of a patient in need thereof a composition as described herein. In another aspect, the present invention provides a method of treating gastroesophageal reflux disease comprising injecting a composition as described herein into the vicinity of the lower esophageal sphincter of a patient. In another aspect, the present invention provides a method for treating or preventing urinary incontinence, comprising administering to a patient in need thereof a composition as described herein, such that the urinary incontinence is treated or prevented. The composition may be administered, for example, periurethrally or transurethrally. In another aspect, the present invention provides a method of treating or preventing fecal incontinence comprising injecting a composition as described herein into the vicinity of the anal sphincter of a patient, such that the fecal incontinence is treated or prevented.

The present invention provides medical implants that comprise a bulking agent. The bulking agent comprises hyaluronic acid and a compound that inhibits degradation of the hyaluronic acid (e.g., aurothiomalate, indomethacin, propylene glycol, heparin, dextran sulphate, fucoidan, and carboxymethyl cellulose). These medical implants may be formulated, e.g., for the management of GERD, fecal incontinence, and urinary incontinence.

In another aspect, medical devices are provided that comprises a medical implant and an inhibitory compound that inhibits degradation of hyaluronic acid. In certain embodiments, medical devices are provided that include an implant that is coated with a composition that includes hyaluronic acid and the inhibitory compound. In one aspect, the present invention provides a medical device, comprising a medical implant, wherein the implant is coated with a composition comprising hyaluronic acid and a gold compound, wherein the gold compound inhibits degradation of hyaluronic acid. In another aspect, the present invention provides a medical device, comprising a medical implant, wherein the implant is coated with a composition comprising hyaluronic acid and indomethacin or an analogue or derivative thereof, wherein the indomethacin inhibits degradation of hyaluronic acid. In another aspect, the present invention provides a medical device, comprising a medical implant, wherein the implant is coated with a composition comprising hyaluronic acid and a sulphate-containing polysaccharide, 5 wherein the sulphate-containing polysaccharide inhibits degradation of hyaluronic acid. The sulphate-containing polysaccharide may be, for example, a fucan such as fucoidan or an analogue or derivative thereof, or dextran sulphate or an analogue or derivative thereof, or heparin or an analogue or derivative thereof. In another aspect, the present invention provides a medical device, comprising a medical implant, wherein the implant is coated with a composition comprising hyaluronic acid and a polymer, wherein the polymer inhibits degradation of hyaluronic acid. In one aspect, the polymer comprises lactic acid residues having the structure (—O—CH(CH₃)—CO—). In another aspect, the polymer comprises ethylene oxide residues having the structure (—OCH₂CH₂—). In another aspect, the polymer comprises poly(lactic acid)-co-poly(ethylene glycol) (PLA-PEG). In another aspect, the polymer comprise poly(L-lactic acid)-co-methoxypoly(ethylene glycol) (MePEG-PLLA) (60:40). In another aspect, the polymer comprises poly(lactic-co-glycolic acid)-co-poly(ethylene glycol) (PLGA-PEG). In another aspect, the polymer comprises poly(caprolactone)-co-poly(ethylene glycol) (PCL-PEG). In another aspect, the polymer is selected from the group consisting of sorbitan esters and copolymers of ethylene oxide and propylene oxide polymers. In another aspect, the polymer is a blend of polymers such as a blend of poly(lactic acid)-co-poly(ethylene glycol) (PLA-PEG) and poly(L-lactic acid)-co-methoxypoly(ethylene glycol) (MePEG-PLLA). In another aspect, the present invention provides a medical device, comprising a medical implant, wherein the implant is coated with a composition comprising hyaluronic acid and a compound such as polyethylene glycol, propylene glycol, an octylphenol ethoxylate, or carboxymethylcellulose (CMC), wherein the compound inhibits degradation of hyaluronic acid. In another aspect, a composition that includes an inhibitory compound (i.e., a hyaluronidase inhibitor) as described herein may further comprise a gold compound (e.g., aurothiomalate), wherein the gold compound inhibits degradation of hyaluronic acid.

These and other aspects of the present invention will become evident upon reference to the following detailed description and drawings.

### BRIEF DESCRIPTION OF THE DRAWINGS

FIG. 1 is a bar graph showing the effect of heparin, aurothiomalate, and indomethacin on the enzyme induced degradation of hyaluronic acid (% viscosity of solution relative to HA control).

FIG. 2 is a bar graph showing the effect of dextran sulphate, fucoidan, heparin, propylene glycol, and indomethacin on enzyme degradation of hyaluronic acid (% viscosity of solution relative to HA control).

FIG. 3 is a bar graph showing the effect of TRITON X-100 on HA degradation by hyaluronidase after overnight incubation.

FIG. 4 is a bar graph showing the effect of various compounds on HA degradation by hyaluronidase.

FIG. 5 is a bar graph showing HA degradation in the presence of heparin sodium salt and hyaluronidase (100 units/ml after 15 hours incubation).

FIG. 6 is a bar graph showing HA degradation in the presence of sodium aurothiomalate and hyaluronidase (100 units/ml after 15 hours incubation).

FIG. 7 is a bar graph showing HA degradation in the presence of CMC and hyaluronidase (100 units/ml after 15 hours incubation).

FIG. 8 is a bar graph showing HA degradation in presence of dextran sulphate and hyaluronidase (100 units/ml after 15 hours incubation).

FIG. 9 is a bar graph showing HA degradation in presence of fucoidan and hyaluronidase (100 units/ml after 15 hours incubation).

### DETAILED DESCRIPTION

Prior to setting forth the invention, it may be helpful to an understanding thereof to set forth definitions of certain terms that will be used hereinafter.

“Hyaluronic acid” or “HA” as used herein refers to all forms of hyaluronic acid that are described or referenced herein, including those that have been processed or chemically or physically modified, as well as hyaluronic acid that has been cross-linked (for example, covalently, ionically, thermally or physically). HA is a glycosaminoglycan composed of a linear chain of about 2500 repeating disaccharide units. Each disaccharide unit is composed of an N-acetylglucosamine residue linked to a glucuronic acid. The molecule can be of variable lengths (i.e., different numbers of repeating disaccharide units and different chain branching patterns) and can be modified at several sites (through the addition or subtraction of different functional groups) without deviating from the scope of the present disclosure.

“Hyaluronidase Inhibitor” or “HI” as used herein refers to a compound that directly or indirectly alters or inhibits the ability of hyaluronidase or other hydrolytic enzyme to hydrolyze hyaluronic acid. “HI” also refers to any molecule that prevents, reduces or extends the time required for the in vivo breakdown of HA, regardless of its specific mechanism of action. Examples of HI's include kaempferol, sulphated β-(1,4)-tetragalactoside, sulphated neomycin, luteolin, myricetin, phloretin, quercetin, sylibin, liquiritigenin, tranilast, baicalein, traxanox, isoliquiritigenin, disodium cromoglycaye, sodium flavonone-7-sulphate, and sodium-5-hydroxyflavone-7-sulphate, gold-containing compounds, indomethacin, sulphated polysaccharides, pharmaceutical co-solvents, non-ionic surfactants, diblock copolymers and carboxymethylcellulose. A variety of compounds and copolymers suitable for use as HI's are described in detail herein.

“Analogue” as used herein refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biological activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diastereomers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

“Derivative” as used herein refers to a chemically or biologically modified version of a chemical compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, the derivative may be more hydrophilic or it may have altered reactivity as compared to the parent compound.

Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH).

The term “derivative” also refers to all solvates, for example hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, lactic acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example, by combining a compound with an inorganic or organic acid or base in a solvent or diluent, or from other salts by cation exchange or anion exchange.

Other types of derivatives include conjugates and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs may be found in, for example, Fleisher et al., Advanced Drug Delivery Reviews 19 (1996) 115; Design of Prodrugs, H. Bundgaard (ed.), Elsevier, 1985; or H. Bundgaard, Drugs of the Future, 16 (1991) 443.

“Inhibit” as used herein refers to an alteration, reduction or abrogation, directly or indirectly, in the activity of an enzyme (e.g., hyaluronidase) relative to a control that is statistically, biologically, or clinically significant.

Any concentration ranges recited herein are to be understood to include concentrations of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. As used herein, the term “about” means±15% of a particular value, range or structure. As used herein, the terms “include” and “comprise” are used synonymously.

As used herein, the terms “average” or “mean” include the arithmetic mean as well as any appropriate weighted averages such as are used in the expression of polymeric molecular weight or particle size distributions.

Various references are set forth herein which, for example, describe in more detail certain procedures or compositions (e.g., compounds, proteins, etc.). These references, including patents and articles, are incorporated by reference in their entirety. It should also be noted that when a PCT application is referred to, it is also understood that the underlying or cited U.S. applications are also incorporated by reference herein in their entirety.

I. Hyaluronic Acid

Hyaluronic acid is a natural substance that is found in the extracellular matrix of many tissues including synovial joint fluid, the vitreous humor of the eye, cartilage, blood vessels, skin and the umbilical cord. Commercial forms of hyaluronic acid having a molecular weight of approximately 1.2 to 1.5 million Daltons (Da) are extracted from rooster combs and other animal sources. Other sources of HA include HA that is isolated from cell culture/fermentation processes. Lower molecular weight HA formulations are also available from a variety of commercial sources.

In general, there are many commercial sources of HA products suitable for use in the present invention, i.e., there are many commercially available HA products to which HI may be added according to the present invention. Examples include commercial compositions for the treatment of osteoarthritis, for viscosupplementation, as ophthalmic viscoelastic products, for facial aesthetics (dermal) and as vesicoureteral reflux implants (bulking agents). More specific details about these and other products are provided below.

HA-containing materials for the intra-articular treatment of pain and other symptoms of osteoarthritis include the following materials. SYNVISC from Genzyme Biosurgery (Ridgefield, N.J.) is an elastoviscous fluid containing hylan [a derivative of sodium hyaluronate (hyaluronan)] polymers derived from rooster combs. ORTHOVISC from Anika Therapeutics (Woburn, Mass.) is a highly purified, high molecular weight, high viscosity injectable form of HA intended to relieve pain and to improve joint mobility and range of motion in patients suffering from osteoarthritis (OA) of the knee. ORTHOVISC is injected into the knee to restore the elasticity and viscosity of the synovial fluid. HYVISC is a high molecular weight, injectable HA product developed by Anika Therapeutics currently being used to treat osteoarthritis and lameness in racehorses. Other HA-based viscosupplementation products for the treatment of osteoarthritis which may be combined with an HI according to the present inventino include HYALGAN from Medexus, Inc. (Canada), SUPARTZ from Seikagaku Corp. (Japan), SUPLASYN from Bioniche Life Sciences, Inc. (Canada), ARTHREASE from DePuy Orthopaedics, Inc. (Warsaw, Ind.), and DUROLANE from Q-Med AB (Sweden).

Viscoelastic solutions of HA have also been used to treat ocular conditions, e.g., as a vitreous substitute during cataract extraction surgery, intraocular lens implantation, retinal reattachment, phacoemulsification surgery, corneal transplantation, and glaucoma surgery. AMVISC and AMVISC PLUS (both from Anika Therapeutics, Inc.) and OCUCOAT (Bausch & Lomb) are high molecular weight, viscoelastic and injectable HA solutions used to maintain eye shape and protect delicate tissues during cataract removal, corneal transplant and glaucoma surgery. HA-based ophthalmic viscoelastic products include PROVIS, VISCOAT, DUOVISC, and CELLUGEL from Alcon Laboratories; HEALON, HEALON G, and HEALON 5 from Pharmacia & Upjohn, VITRAX from Allergan; BIOLON from Bio-Technology General; STAARVISC from Anika Therapeutics/Staar Surgical; SHELLGEL from Anika Therapeutics/Cytosol Opthalmics; and UNIVISC from Novartis.

HA-based products can also be used as bulking agents. Here the material is injected into a tissue to restore volume, provide support and restore function—typically to “bulk” the tissue surrounding an incontinent sphincter. HA-based bulking agents are used in the treatment of urinary incontinence, fecal incontinence and gastro-esophageal reflux; all conditions where leakage occurs as a result of an inefficient or damaged sphincter muscle. A representative example of a HA-based vesicoureteral reflux (urinary incontinence) product for use in the present invention is DEFLUX from Q-Med/Priority Healthcare.

Hyaluronic acid products are also used to prevent adhesions following a variety of surgical procedures. Adhesions are connections or bridges of scar tissue that occur between adjacent tissues that are damaged during surgery. Adhesion scar tissue can impair normal anatomical function and can lead to innumerable clinical problems including pain, bowel obstruction, infertility and nerve root entrapment. The INCERT family of bioabsorbable, cross-linked hyaluronic acid (HA) products is designed to be placed between adjacent tissues during surgery to act as a barrier to inhibit the formation of scar tissue. Other HA-based surgical adhesion products include GYNECARE INTERGEL (LifeCore), and SEPRAFILM adhesion barriers from Genzyme Biosurgery, Inc. (Cambridge, Mass.).

Other HA products include implants made for use during orthopedic surgery for the purpose of filling deficits, preventing scarring, providing tissue support and accelerating healing. For example, OSSIGEL is a viscous formulation of hyaluronic acid (HA) and basic fibroblast growth factor (bFGF) designed to accelerate bone fracture healing (Orquest, Inc.).

Perhaps the most rapidly growing area of use of HA products in medicine is in body augmentation (e.g., facial), wrinkle treatments and other cosmetic or aesthetic applications. In cosmetic procedures, HA is typically injected into the subcutaneous tissue to fill in skin depressions and defects in order to reduce the appearance of lines or other unwanted marks. Manufactured synthetic hyaluronic gels commercially available for this purpose include HYLAFORM (also known as HYLAN B from Genzyme Biosurgery; RESTYLANE and PERLANE (from Q-Med AB, Sweden). MACROLANE (Q-Med) is a product in development for breast augmentation.

Other applications that utilize HA-containing materials include drug delivery, cancer therapy, and the treatment of interstitial cystitis. Examples of HA-containing materials for use in drug delivery and which may be combined with an HI include Hyaluronic Induced Targeting (HIT) (SkyePharma (UK)) and NASHA gel (Q-Med). Topical formulations, such as SOLARESE and SOLARASE from Meditech (Australia), are topical gels used in the treatment of skin cancer. HA-based materials, which are commercially available for use in the treatment of interstitial cystitis, include CYSTISTAT (Bioniche Life Sciences, Inc.), a sterile sodium hyaluronate solution for the temporary replacement of the glycosaminoglycan (GAG) layer on the bladder epithelium.

Thus, both refined HA, and compositions containing HA, are readily available on the commercial market for a wide variety of clinical indications. These HA-containing materials are examples of the HA-containing materials that may be used in the present invention as the source of HA.

II. Hyaluronidase Inhibitors

A variety of compounds can be used to inhibit or reduce the enzymatic degradation of HA in vivo and are suitable for use in the practice of this invention. For example, compounds can be combined with HA to produce an HA implant that resists degradation and has prolonged activity in a variety of clinical indications.

A hyaluronidase inhibitor (HI) may be used to inhibit the degradation of hyaluronic acid in vivo at concentrations in the micro- to millimolar range. These compounds may be delivered simultaneously or sequentially with the hyaluronic acid. For example, the HI may be delivered to the patient simultaneously with the hyaluronic acid by incorporating the hyaluronic acid into the administered formulation. Alternatively, or in addition, these HI compounds may be administered after hyaluronic acid administration. In certain aspects, continuous exposure of target tissue to these compounds via controlled release from polymeric dosage forms of these compounds may be preferred. These compositions and other combinations are described in further detail herein.

1. Gold Compounds

A variety of gold compounds can function as inhibitors of HA breakdown in vivo and are suitable for use in the practice of this invention. Gold compounds, as used herein, include complexes in which gold is chelated or bound to one or more ligands, organo-gold compounds, inorganic gold compounds and salts thereof, and elemental (e.g., metallic) gold. The compounds may be hydrophilic, hydrophobic, amphiphilic, and may be dissolved in solution or in the form of a particle suspension (e.g., colloidal gold). At times organometallic compounds can be toxic—a person of skill in the art will know how to determine what amount to use such that these compounds will not be toxic to the subject receiving the treatment. Examples of gold compounds that can be combined with HA to produce an HA-gold implant that resists degradation and has prolonged biological activity in a variety of clinical indications are described below.

a. Gold (I) Complexes

In one aspect, the gold compound is a gold (I) complex. Gold complexes include compounds in which gold (I) is chelated, bound, complexed, or otherwise joined to one or more ligands (e.g., coordination complexes). Representative examples of such gold(I) complexes include gold (I) phosphine compounds, gold (I) phosphine or phosphate thiolates, bis-coordinated gold (I) salts, and gold (I) chelates (see, e.g., U.S. Pat. No. 5,527,779)

Gold (I) phosphines and related compounds have the general formula: R₃PAuX, wherein R is alkyl (e.g., methyl, ethyl, isopropyl, or n-butyl), aryl, or heterocyclic or a substituted derivative thereof, and X is halogen. Representative examples of gold (I) phosphine compounds include, for example, triphenylphosphine complexes (Ph₃PAuCl) and Et₃PAuCl.

Another example has the general formula R₃PAuX, wherein X is imidazole or X is a 2-thiazolinyl, thio-2-benzimazolyl or 2-benzoxazolylthio-moiety.

Other examples of gold (I) phosphine compounds include NSC652537, NSC652539, 2-coordinate triphenyl phospine gold (I) complexes with AuSP and AuNP cores (see, e.g., Nomiya, K., et al. J. Inorg. Biochem. 2003; 95(2-3): 208-20), complexes containing mono- and diphosphine derivatives, e.g., chlorotriphenylphosphine-1,3-bis(diphenylphosphine)propanegold(I) (see, e.g., Caruso, F. J. Med. Chem. 2003; 46(9): 1737-42), triphenylphospine complexes having nitrogen containing heterocycles, such as pyrazole and imadazole (see, e.g., Nomiya, K., et al.; J. Inorg. Biochem. 2000 March; 78(4): 363-70), chloro(triethylphosphine)gold(I) (TEPAu) (Et₃PAuCl) and phosphonate complexes (and phosphine reaction products), and tetrakis((trishydroxymethyl)phosphine) gold(I) chloride (see, e.g., Pillarsetty, N., et al. J. Med. Chem. 2003; 46(7): 1130-1132).

Examples of related compounds include trialkyl phosphite gold compounds having the general formula (RO)₃PAuX and thiocyanate gold complexes having the formulae: R₃PAuSCN and (RO)₃PAuSCN, wherein R is alkyl (e.g., methyl, ethyl), aryl (e.g., phenyl), or heterocyclic and may be substituted or unsubstituted, and X is halogen. Gold (I) phosphine (or phosphite) thiolates include those compounds having the general formula: R₃PAuSR, wherein R is alkyl (e.g., ethyl), alkoxyl, or phenyl, and R′ is H, alkyl, aryl, or heterocyclic and may be substituted or unsubstituted. For example, R′ may be a substituted carbohydrate moiety resulting in compounds having the general structure: 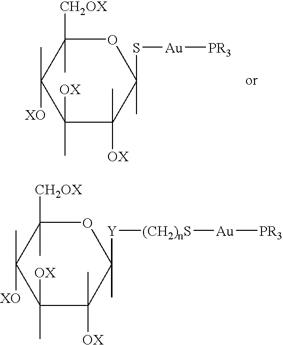
wherein X is H, acetyl, or formyl; Y is O or S; and n is 1-12.

An example of a gold phosphine compound is auranofin (1-Thio-β-D-glucopyranose-2,3,4,6-tetraacetato-S)(triethylphosphine)gold), which is known to possess anti-inflammatory and anti-rheumatic properties.

Other examples of phosphine or phosphite Au(I) thiolates include: 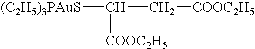
and phosphine or phosphate Au(I) complexes including derivatives of thioalcohols, thioacids, and thiophenols, and those having the general formula R₃PAuX, wherein X is 2-thiazolinyl, thio-2-b enzimazolyl, and 2-benzoxazolylthio-, those having the general formula: R₃PAu)₂ S, and large ring chelates compounds such as the following also may be used 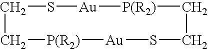
wherein R is H, alkyl, aryl, or heterocyclic and may be substituted or unsubstituted.

Other examples of gold chelates include the large ring gold chelates described in Weinstock et al., J. Med. Chem. 17(1): 139-140, 1974.

Examples of bis-coordinated gold (I) salts include those having the following general formulae: [R₃PAuPR₃]⁺X⁻; [R₂SAuSR₂]⁺X⁻; [RC₅H₄NAuNC₅H₄R]⁺X⁻; and [R₃PAuNC₅H₄R]⁺X⁻, wherein R is alkyl, aryl or heterocyclic and can be either substituted or unsubstituted; and X is halide, ClO₄, BF₄ or any monovalent or divalent anion known in the art.

Representative examples of gold (I) chelates have the following formula: 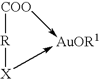
wherein R is any suitable bridging moiety and may be substituted or unsubstituted alkyl, aryl or heterocyclic; X is O, N or SO₂ NR₂ and R¹ is H, alkyl, aryl or heterocyclic and may be substituted or unsubstituted. For example, R may be C₆H₄, X is O and R¹ is C₂H₅.

b. Organo-Gold Compounds

In another aspect, the gold compound is an organo-gold compound. A variety of organo-gold compounds, including those described herein, may be used in the present compositions.

In one aspect, the organo-gold compound may possess anti-inflammatory and anti-rheumatic properties (e.g., also referred to as chrisotherapeutic compounds). Representative examples of chrisotherapeutic gold compounds include auranofin (described above), gold thiopolypeptide, and aurothiomalate and sodium aurothiomalate (MYOCRISIN; butanedioic acid, mercapto-, monogold(1+) sodium salt (9CI)) (see J. Reprod. Fertil. 1980; 60(2):461-7), which has the following general structure:

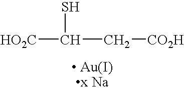

Other examples of organo-gold compounds include aurothioglucose (1-Thio-D-glucopyranosato-O2,S1)gold and derivatives thereof, such as bis(thioglucose) gold (I), bis(thiomalate) gold (I), and Aurate(1-){[3-[[2-propenylamino)thioxomethyl]imino] benzoate(2-)]-, sodium}] (NSC617746 sodium salt), and gold(I) complexes including pyridine derivatives, such as NSC689418 and NSC689419, imidazole derivatives, such as clotrimazole and ketoconazole (see, e.g., Navarro, N., et al. Inorg. Chem. 2001; 40(27): 6879-84), NSC652538, and dinuclear gold(I) dithiophosphonate complexes (see, e.g., Maspero, A., et al. Inorg. Chem. 2003; 42(17): 5311-9).

c. Gold (III) Complexes

In another aspect, the gold compound is a gold (III) complex. Representative examples of gold (III) complexes include cholylglycinato complexes, such as chlorobischolylglycinatogold(III) (see, e.g., Carrasco et al., J. Inorg. Biochem. 84(3-4): 287-92, 2001), tri- and tetradentate phosphinothiolate complexes (e.g., Ortner et al., Inorg. Chem. 39(13): 2801-6, 2000), and complexes with ethylenediamine, diethylenetriamine, tetraazacylotetradecane, 2, 2; -bipyridine, 6-(1,1-dimethylbenzyl)-2,2′-bipyridine), cyclam, phenanthroline, terpyridine ligands (see, e.g., Messori et al., J. Med. Chem. 43(19): 3541-8, 2000 and Marcon et al., Eur. J. Biochem. 270(23): 4655-61, 2003).

d. Inorganic Gold Compounds

In another aspect, the gold compound is an inorganic gold compound. Representative examples of inorganic gold compounds include gold III and IV chloride (AuCl₃ and AuCl₄, respectively) and gold salts, such gold (II) chloride, hydrochloride, sodium gold(III) chloride, and gold sodium thiosulphate.

e. Gold Particles

In another aspect, the gold is in the form of particles. Metallic gold particles having an average size of below about 50 nm may be referred to as “colloidal gold”. Colloidal gold preparations, which are generally in the form of liquid suspensions, include gold particles that range from about 0.5 nm to about 40 nm or less than about 10 nm, or about 1 nm to about 3 nm. Colloidal gold particles may be prepared using methods known to those skilled in the art and are commercially available.

Gold particles may be functionalized or non-functionalized. Functionalized gold particles may be conjugated to compounds such as, e.g., oligonucleotides, lipids, peptides, proteins, enzyme inhibitors, antibodies, or other compounds having a suitable reactive moiety.

A variety of functionalized and non-functionalized gold particles are available from Nanoprobes, Inc. (Yaphank, N.Y.). Positively and negatively charged NANOGOLD particles (1.4 nm) may be used in the present compositions. The positive NANOGOLD has multiple amines on its surface, whereas negative NANOGOLD has multiple carboxyl groups. Functionalized NANOGOLD particles may be conjugated to a variety of biological compounds. For example, monomaleimido NANOGOLD may be used to covalently label Fab′, IgG, proteins or peptides containing cysteine, and other molecules with sulfhydryls. A similar product, monomaleimido undecagold (also from Nanoprobes, Inc.) has a core of 11 gold atoms only 0.8 nm in diameter with single maleimide group, for selectively labeling thiols (—SH). Mono-Sulfo-NHS-NANOGOLD includes a sulfo-N-hydroxysuccinimide ester (sulfo-NHS) that reacts with primary amines for covalent attachment to a protein, lipid, peptide, modified oligonucleotide or other amine-containing molecule. A similar product based on the undecagold compound is also available (Mono-Sulfo-NHS-Undecagold). NANOGOLD particles are also available with a primary amine attached for other crosslinking reactions (e.g., covalent attachment to the carbohydrate moiety of a glycoprotein). Cationic gold particles resulting from the conjugation of colloidal gold particles with poly-L-Lysine are available from Energy Beam Services (Agawam, Mass.) under the trade name BIOSITE.

Gold compounds such as, for example, aurothiomalate, may be used to inhibit the degradation of hyaluronic acid. These compounds may be delivered simultaneously or sequentially with the hyaluronic acid. For example, the gold compound may be delivered to the patient simultaneously with the hyaluronic acid by incorporating the gold compound into the administered formulation. Alternatively, or in addition, these compounds may be administered after hyaluronic acid administration. In certain aspects, continuous exposure of target tissue to gold compounds via controlled release from polymeric dosage forms of these compounds may be preferred.

2. Polysaccharides

In one aspect, the hyaluronidase inhibitor may be a polysaccharide or a sulphated (i.e., sulphate-containing) polysaccharide or an analogue or derivative of a sulphated polysaccharide. Representative examples of polysaccharides include alginic acids, pectins, and glycosaminoglycans (see, e.g., Biosci. Biotechnol. Biochem. 1997; 61(6):1030-2, J. Enzyme Inhib. Med. Chem. 2002; 17(3): 183-6). Representative examples of sulfonated compounds include sulfonated β-(1,4)-galacto-oligosaccharides (n=2-6) with degrees of sulfonation from 0.2 to 1, sulfonated neomycin, O-sulfonted HA (see, e.g., Arch. Biochem. Biophys. 1999; 370(2): 176-82), sulfonated planetose, sulphated hydrochinone diglalctoside, sulphated 2-hydroxy phenyl monolactobioside. Representative examples of sulphated polysaccharides include heparin/heparan sulphate (see, e.g., Arch. Biochem. Biophys. 1999; 370(2): 176-82; Matrix Biol. 2002; 21(1):31-7), dextran sulphate, and fucans (e.g., fucoidan).

Dextran sulphate is a polyanion that is freely soluble in water, which can interact with cations and polycations. Dextran sulphate, therefore, is capable of binding to various membranes, particularly those having a positive charge. Dextran sulphate has been reported to have a variety of clinical uses. Dextran sulphate and derivatives have been shown to inhibit cancer cell growth (Bittoun P., Carbohydrate Research 1999 (3-4) p 247-255); to have anticoagulant effects (Mauray S., 1998 J Biomat. Sci. Poly ed. 1998 9 4 p 373-87); to prevent the formation of syncytia or clumping of white blood cells which occurs in AIDS patients; and can act as a stabilizer (pharmaceutical excipient) for sensitive natural ingredients.

Heparin is a heterogeneous group of straight-chain anionic mucopolysaccharides called glycosaminoglycans having anticoagulant properties. Its principal active component is a glycosaminoglycan composed of D-glucuronic acid and D-glucosamine (both sulphated) in a 1,4-α linkage having molecular weight of about 6000-20,000, depending on the method of preparation and the source. Different heparin samples may have varying levels of N- and O-sulphation within the hexosamine and hexuronic acid residues. Heparin (including derivatives thereof) is widely used as an anticoagulant in numerous vascular scenarios in which blood clotting may be an issue (e.g., open heart surgery and dialysis).

Fucans (including fucoidan) are high molecular weight, sulphated polysaccharides extracted from brown seaweeds. These compounds have multiple inhibitory actions in vivo and in vitro including anti-thrombin, anti-proliferative, anti-complement, anti-cancer and anti-neutrophil migration effects (Riou D et al, Anticancer Research, 16 (3A): 1213-1218, 1996; Itoh, Anticancer Research 13 (6A): 2045-2052, 1993; Nishiro et al., Thromb. Res. 62: 765-773, 1991; Blondin et al., Mol. Immunol. 31: 247-253, 1994; Patankar et al., J. Biol. Chem. 268: 21770-21776, 1993. Fucoidan also has been marketed as a health food and has been proposed as a cosmetic or dermal agent (see, e.g., JP 01031707 and JP 01085905).

Sulphated polysaccharides such as heparin, heparan sulphate, dextran sulphate, and fucoidan may be used to inhibit the degradation of hyaluronic acid. These compounds may be delivered simultaneously or sequentially with the hyaluronic acid. For example, the sulphated polysaccharide may be delivered to the patient simultaneously with the hyaluronic acid by incorporating the sulphated polysaccharide into the administered formulation. Alternatively, or in addition, these compounds may be administered after hyaluronic acid administration. In certain aspects, continuous exposure of target tissue to sulphated polysaccharides via controlled release from polymeric dosage forms of these compounds may be preferred.

3. Copolymers of PLA PLGA and Other Materials

In another aspect, the hyaluronidase inhibitor (HI) may be a polymeric material. The polymer may be a homopolymer or a copolymer (e.g., a diblock or triblock copolymer). In one aspect, the polymer may be a homopolymer such as poly(lactic acid) (PLA). In another aspect, the polymer is a diblock copolymer. A variety of diblock copolymers function as inhibitors of HA breakdown in vivo and are suitable for use in the practice of this invention.

Examples of diblock copolymers that can be combined with HA to produce an HA-diblock copolymer implant that resists degradation and has prolonged biological activity in a variety of clinical indications include diblock copolymers of lactic acid and/or glycolic acid, and poly(ethylene glycol). In one aspect, the copolymer may include lactic acid residues having the structure (—O—CH(CH₃)—CO—), residues of ethylene oxide having the structure (—OCH₂CH₂—), residues of glycolic acid (—O—CH₂—CO—), or residues of caprolactone (—O—(CH₂)₅—CO—). For example, the diblock copolymer may be poly(lactic acid)-co-poly(ethylene glycol) (PLA-PEG); poly(lactic-co-glycolic acid)-co-poly(ethylene glycol) (PLGA-PEG); and poly(caprolactone)-co-poly(ethylene glycol) (PCL-PEG). In one aspect, the polymer is a copolymer having a 60:40 ratio of methoxy poly(ethylene glycol) and poly(L-lactic acid) (MePEG-PLLA). In all of these copolymers, methoxy poly(ethylene glycol) (MePEG) may be substituted for PEG. In one aspect, the copolymer is poly(L-lactic acid)-co-methoxy poly(ethylene glycol) (PLA-MePEG).

The monomers within the diblock copolymer may be arranged randomly in the chain or may be chains of individual polymers linked together. Such linked copolymers frequently are manufactured using a combination of a hydrophobic polymer and a hydrophilic polymer. For example, PLA which is hydrophobic in nature may be used in combination with poly(ethylene glycol), which is hydrophilic in nature. The resulting amphipathic copolymer will contain both hydrophilic and hydrophobic zones. Such molecules are frequently utilized in the pharmaceutical industry as they may associate through either zone with drugs molecules to modify their behavior. Amphipathic copolymers also may be used as blending agents with other polymers to modify the overall behavior of the main polymer. Such properties may vary the ability of the diblocks to form micelles and solubilize non-water soluble drugs or to plasticize rigid polymers like PLGA so they are more biocompatible and may release encapsulated drug more rapidly (see, e.g., Liggins, R. T., et al., Advanced Drug Delivery Reviews (2002) 54, p 191-202; Kwon G., et al. (1995) 16, p 295-309; and Jackson, J. K., et al. (2004), Int. Journal of Pharmaceutics (in press)).

In one aspect, the HI is a copolymer of poly(ethylene oxide) or poly(ethylene glycol). The structure of PEG and PEO are the same, with PEG usually referring to polymers of less than 20,000 molecular weight and PEO referring to polymers with larger molecular weights. Considerable research effort has focused on combining poly(ethylene glycol) (PEG) or poly(ethylene oxide) (PEO) with PLA or PLGA to produce a copolymer that includes the hydrophilic and biocompatible nature of PEG and the degradable properties of PLGA. Depending on the composition of the copolymer, the characteristics of the resulting polymer can be varied from hydrophilic to hydrophobic and from non-degradable to degradable.

Some of the earliest work in developing block copolymers of PLA₁₀₀ and poly(ethylene oxide) involved varying the amount of PEO in the copolymer, such that the equilibrium water content of the polymer matrix may reach more than 60% (Cohn and Younes in 1988). For these particular copolymers, the lactic acid portion ranged from 20 to 84 mol % and the PEO chains had MW ranging from about 600 to 6000. Other work with random block copolymers of PLA and PEG has evaluated the degradation behavior of these materials and their utility as microparticles for drug delivery. Degradation rates appear to be strongly dependent on the PEG content, with partially degraded PLA segments sometimes being solubilized by attached PEG before they may otherwise have been released from the bulk polymer.

In one aspect, the copolymer includes a PEG or PEO central block and PLA chains at either end. These polymers may be prepared by starting with a PEG segment of a given length and then polymerizing the PLA while using the PEG as the initiator for the polymerization reaction. The length of the PEG block as well as the length of the PLA₁₀₀ blocks may have an effect on water absorption and degradation of these copolymers. A series of papers by Kissel have explored the synthesis of these triblock materials, in vitro degradation, drug delivery, in vitro biocompatibility, in vivo biocompatibility, as well as the microenvironment of PLA-PEO-PLA microparticles during degradation. The biocompatibility studies have shown that PLA-PEO-PLA polymers show very similar and minimal adverse tissue reactions. Drug delivery studies that compared in vitro delivery of BSA from microparticles prepared from PLA₁₀₀-PEO-PLA₁₀₀ and PLA₅₀-GA₅₀-PEO-PLA₅₀-GA₅₀ polymers showed that the PLAGA-containing polymers exhibited fairly continuous release while PLA-containing polymers had two phases of release more typical of PLAGA microparticles. Release studies of cytochrome C and FITC-dextran from PLA₅₀-GA₅₀-PEO-PLA₅₀-GA₅₀ microparticles also showed continuous release in vitro.

In another aspect, the HI includes monomeric units derived from ε-caprolactone. For example, the HI may be a copolymer of 6-caprolactone with PLA or PGA. A number of research groups have investigated copolymers of PLA or PGA with 6-caprolactone. In one study, a series of 66 different terpolymers of DL-lactide, glycolide, and ε-caprolactone to determine the degradation rates and other properties of cast films (Sawhney and Hubbell). They found that the longest degradation times were for polymers with a 2:1:7 ratio of glycolide lactide 6-caprolactone and the fastest degradation for polymers with a 6:3:1 ratio. The physical properties of copolymers of lactide and ε-caprolactone have been found to vary from hard to rubbery as the ε-caprolactone content increased from 5 to 20 wt %. Porous copolymers with 50% ε-caprolactone content have been evaluated as implants for meniscal tissue regeneration in the knee joint. The polymers showed a bulk degradation behavior, and, during degradation, separated into a crystalline phase containing mainly L-lactide and an amorphous phase composed mainly of ε-caprolactone.

In yet another aspect, the copolymer includes monomeric units derived from glycine, p-hydroxybenzoic acid and p-hydroxycinnamic acid, or aspartic acid. For example, the copolymer may be a copolymer including monomeric units derived from glycine, p-hydroxybenzoic acid and p-hydroxycinnamic acid, or aspartic acid and PLA or PGA. These materials are biodegradable materials having degradation and release properties that differ from PLA or PGA alone.

These diblock copolymers (for example, poly caprolactone-co-PEG or PLA-PEG) may be used to inhibit the degradation of hyaluronic acid by simply blending or dissolving these agents into the administered hyaluronic acid formulation. Alternatively they may be administered after the hyaluronic acid was administered.

The polymers and copolymers (e.g., diblock copolymers) described above may be used to inhibit the degradation of hyaluronic acid. These compounds may be delivered simultaneously of sequentially with the hyaluronic acid. For example, the polymer may be delivered to the patient simultaneously with the hyaluronic acid by incorporating the hyaluronic acid into the administered formulation. Alternatively, or in addition, these compounds may be administered after hyaluronic acid administration. In certain aspects, continuous exposure of target tissue to these compounds via controlled release from polymeric dosage forms of these compounds may be preferred.

4. Pharmaceutical Excipients

In another aspect, the HI may be a pharmaceutical excipient. As used herein, a pharmaceutical excipient refers to an additive that is used to convert pharmacologically active compounds into dosage forms suitable for administration to patients.

Excipients may be used to improve bioavailability and bioequivalence of pharmaceutical agents. The excipients used in formulating dosage forms include, without limitation, fillers, binders, disintegrating agents, lubricants, coatings, solvents, suspending agents, and dyes. These excipients are useful in that they have high degrees of biocompatibility so they perform their role in improving the formulation characteristics of drugs without inducing any unwanted toxicity in patients.

Examples of excipients suitable for use as HI's include carboxymethylcellulose (CMC) sodium, selected triblock polymers of propylene oxide and ethylene oxide (a series of these compounds are commercially available under the tradenames PLURONIC and PLURONIC R from BASF Corporation, Mount Olive, N.J.), and polyethylene glycol (PEG).

Polyethylene glycol has been used in a variety of physical forms depending on the application and the desired delivery form. Solid PEGs are useful as water-soluble ointment bases. In aqueous vehicles, PEGs can be used to adjust viscosity and consistency. When used in conjunction with other emulsifiers, PEGs can act as emulsion stabilizers. Liquid PEGs are used as water-miscible vehicles for the contents of soft gelatin capsules. The aqueous solubility or dissolution characteristics of poorly soluble compounds can be enhanced by making solid dispersions with an appropriate PEG. Higher molecular weight PEGs can enhance the effectiveness of tablet binders and impart plasticity to granules. When used for thermoplastic granulations, a mixture of the powdered constituents with 10 to 15% PEG 6000 is heated to 70-75° C. The mass becomes paste-like and forms granules if stirred while cooling. The technique is useful for dosage forms such as lozenges when prolonged disintegration is required. PEG's have been used as plasticizers in film coatings. Solid grades can be used alone for film coating tablets, and can be useful as hydrophilic polishing materials. They are widely used as plasticizers in conjunction with film-forming polymers. The presence of PEGs, especially the liquid grades, in films tends to increase their water permeability, and may reduce protection against low pH in enteric coating films. PEGs are useful as plasticizers in micro-encapsulated products to avoid rupture of the coating film when microcapsules are compressed into tablets. Grades of PEG 6000 and above can be used as lubricants, particularly in soluble tablets. The lubricant action is not as good as that of magnesium stearate, and stickiness may develop if the material becomes too warm during compression. An anti-adherent effect is also exerted, again subject to avoidance of over-heating.

In another aspect, the pharmaceutical excipient is a sorbitan ester (SPAN), such as SPAN 20, SPAN 40, and SPAN 85 (Adolor Corporation, Exton, Pa.).

In yet another aspect, the pharmaceutical excipient is a polysorbate compound such as a TWEEN (ICI Americas Inc., Bridgewater, N.J.) which are typically used as oil-in-water emulsifying agents and in the preparation of emulsions, creams, ointments and suppository bases. TWEEN's are polyoxyethylene derivatives of sorbitan esters. The presence of polyoxyethylene chains makes these derivatives hydrophilic. Polysorbates are well-tolerated when taken orally, with very low levels of toxicity, and practically irritation-free topically.

In yet another aspect, the pharmaceutical excipient is propylene glycol, which is widely used as a solvent, extractant, and preservative.

Pharmaceutical excipients such as, but not limited to, propylene glycol, carboxymethylcellulose, PLURONIC, and SPAN may be used to inhibit the degradation of hyaluronic acid. These compounds may be delivered simultaneously of sequentially with the hyaluronic acid. For example, the polymer may be delivered to the patient simultaneously with the hyaluronic acid by incorporating the hyaluronic acid into the administered formulation. Alternatively, or in addition, these compounds may be administered after hyaluronic acid administration. In certain aspects, continuous exposure of target tissue to these compounds via controlled release from polymeric dosage forms of these compounds may be preferred.

5. Other Hyaluronidase Inhibitors

Other exemplary compounds that can be combined with HA to produce an HA implant that resists degradation and has prolonged activity in a variety of clinical indications include flavinoids, anti-inflammatory agents and surfactants, or any combination thereof. The listed compound categories are not mutually exclusive—compounds may fall under more than one category, as is known in the art (e.g., glychyrrhizin may be both a favonoid and an anti-inflammatory agent).

In one aspect, the HI may be a flavonoid. Flavonoids are polyphenolic compounds that are ubiquitous in nature and are categorized, according to chemical structure, into flavonols, flavones, flavanones, isoflavones, catechins, anthocyanidins, chalcones, and neoflavonoids. Flavonoids are known to be found in higher plants, such as fruits and vegetables, and in beverages (e.g., tea, coffee, beer, wine, fruit drinks). Exemplary flavonoids useful as an HI as described herein include condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone, or the like. See, e.g., Matrix Biol. 2002, 21(1):31-7; Biol Reprod. 1997, 56(6):1383-9; Experientia 1991, 47(11-12):1196-200; Biochem Pharmacol. 1990; 40(2):397-491.

In another aspect, the HI may be a phenolic compound. Representative phenolic compounds include diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B.

In another one aspect, the HI may be an anti-inflammatory agent, which may be steroidal or non-steroidal. Representative anti-inflammatory agents include indomethacin (see, e.g., Matrix Biol. 2002; 21(1):31-7), aescin, traxanox, salicylates (see, e.g., Matrix Biol. 2002; 21(1):31-7), eicosatrienoic acid (see, e.g., J. Enzyme Innib. Med. Chem. 2002; 17(3):183-6), glychyrrhizin (see, e.g., Biol. Pharm. Bull. 1997; 20(9): 973-7); agents that modulate allergic reactions such as disodium cromoglycate (DSCG), tranilast, liquiritigenin, isoliquiritigenin, baicalein (see, e.g., Chem. Pharm. Bull. 1992; 40(6):1439-42; Toxicon. 2003; 42:635-646); sodium polystyrene sulfonate (N-PSS) (see, e.g., J. Androl. 2000; 21(6):862-75); saccharic acid (see, e.g., J. Enzyme Inhib. Med. Chem. 2003; 18(4):377-382); chondroitin sulphate A-derived oligosaccharide (ChSAO) (see, e.g., Biol. Reprod. 2005; 72(4): 1061), phenylbutazone, oxyphenbutazone, γ-linolenic acid, fenoprofen, or the like.

In one aspect, the HI may be indomethacin. Indomethacin is a non-steroidal, anti-inflammatory, analgesic, and antipyretic agent used in the management of rheumatoid arthritis, osteoarthritis, and gout. In yet another aspect, the HI may be a surfactant such as tetradecyl sodium sulphate (see J. Reprod. Fertil. 1983; 68(2):257-63), or octylphenol ethoxylate, sold under the trade name TRITON X-100 (Dow Chemical Co., Midland Mich.) and which is a non-ionic surfactant. Indomethacin and TRITON-X 100 may be used to inhibit the degradation of hyaluronic acid in vivo. These compounds may be delivered simultaneously of sequentially with the hyaluronic acid. For example, the polymer may be delivered to the patient simultaneously with the hyaluronic acid by incorporating the hyaluronic acid into the administered formulation. Alternatively, or in addition, these compounds may be administered after hyaluronic acid administration. In certain aspects, continuous exposure of target tissue to these compounds via controlled release from polymeric dosage forms of these compounds may be preferred.

Other exemplary compounds that can be combined with HA to produce an HA implant that resists degradation and has prolonged activity in a variety of clinical indications include ascorbic acids, such as Vitamin C or L-ascorbic acid 6-hexadecanoate (J. Biol. Chem. 2004, 279(44):45990-97); saponins (see, e.g., J. Enzyme Inhib. Med. Chem. 2002; 17(3):183-6) such as hederagenin; cysteamine; echinacea; rosmaric acid; guanidine hydrochloride; L-arginine.

III. Formulations

The compositions of the present invention can be prepared in a variety of ways. For example, an HI, such as aurothiomalate or fucoiden, can be dissolved or suspended directly into an HA solution. If the compound is stable in the HA solution, the composition containing the HA and the compound can be prepared in a single application apparatus. If the compound is not stable in the HA solution for a significant length of time, the composition can be made as a two-component system in which the components are mixed immediately prior to use.

The sulphated polysaccharides (such as dextran sulphate, heparin and fucoidan and analogues or derivatives thereof) are generally water soluble and may be co-administered as a solution with hyaluronic acid. In some cases, these materials may be administered as solids at the time of hyaluronic acid application. Delivery in a solid form may be practical when delivery occurs in the presence of an exposed surgical site. Alternatively, these compounds may be injected or applied in suspension in a non-aqueous injection vehicle (carrier). The injection may occur before, at the same time, or after the hyaluronic acid administration.

Pharmaceutical excipients such at polyethylene glycol, propylene glycol, SPAN, and PLURONICs, as well as the diblock copolymers and TRITON X-100 are generally water soluble and may be co-administered as a solution with hyaluronic acid. In some cases these material may be administered as solids at the time of hyaluronic acid application in the case for example of exposed surgical sites. Alternatively they may be injected or applied in suspension in a non aqueous injection vehicle. This injection may occur at the before, at the same time or after the hyaluronic acid administration. One particular advantage of the pharmaceutical excipients as well as the diblock copolymers and TRITON X-100 is that they themselves may make excellent injection vehicles for hyaluronic acid whereby the hyaluronic acid is dissolved or suspended in these agents for injection of application to the appropriate site. For example the diblock copolymers based on PCL-PEG are waxy materials at room temperature and gentle warming allows them to become viscous liquids which may easily be injected into the body.

In one aspect, the hyaluronidase inhibitors may be placed in a carrier. The carrier may serve as a vehicle for delivery of the HI composition to a desired location and may impart other desirable properties to the composition (e.g., hydrophilicity, bioavailability, viscosity, and the like).

Representative examples of carriers include both polymeric and non-polymeric carriers (e.g., liposomes or vitamin-based carriers), which may be either biodegradable or non-biodegradable. Representative examples of biodegradable polymers include albumin, gelatin, starch, cellulose, dextrans, polysaccharides, fibrinogen, poly(esters) (e.g., poly (D,L lactide), poly (D,L-lactide-co-glycolide), poly (glycolide), poly(ε-caprolactone), copolymers and blends thereof), poly (hydroxybutyrate), poly (alkylcarbonate), poly(anhydrides) and poly (orthoesters) (see generally, Illum, L., Davids, S. S. (eds.) “Polymers in controlled Drug Delivery” Wright, Bristol, 1987; Arshady, J., Controlled Release 17:1-22 (1991); Pitt, Int. J. Pharm 59:173-196 (1990); Holland et al., J. Controlled Release 4:155-0180 (1986)). Representative examples of non-biodegradable polymers include block copolymers based on ethylene oxide and propylene oxide (i.e., copolymers of ethylene oxide and propylene oxide polymers), such as the family of PLURONIC polymers available from BASF Corporation (Mount Olive, N.J.), EVA copolymers, silicone rubber, poly(methacrylate) based and poly(acrylate) based polymers. In certain embodiments, the polymers may be poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of caprolactone and/or lactic acid, and/or glycolic acid with polyethylene glycol or methoxypolyethylene glycol and blends thereof.

Polymeric carriers (polymers) may be fashioned in a variety of forms, including for example, rod-shaped devices, pellets, slabs, or capsules (see, e.g., Goodell et al., Am. J. Hosp. Pharm. 43:1454-1461 (1986); Langer et al., “Controlled release of macromolecules from polymers”; in Biomedical polymers, Polymeric materials and pharmaceuticals for biomedical use, Goldberg, E. P., Nakagim, A. (eds.) Academic Press, pp. 113-137, 1980; Rhine et al., J. Pharm. Sci. 69:265-270 (1980); Brown et al., J. Pharm. Sci. 72:1181-1185 (1983); and Bawa et al., J. Controlled Release 1:259-267 (1985)). These hyaluronidase inhibitors may be linked by occlusion in the matrices of the polymer, bound by covalent linkages, or encapsulated in microcapsules. Within certain embodiments of the invention, the hyaluronidase inhibitor containing compositions are provided in non-capsular formulations such as microspheres (ranging from nanometers to micrometers in size), pastes, gels, threads of various size, films, meshes, and sprays. In certain embodiments, the composition is in a form that is suitable for injection into a desired location in a patient.

In certain emdodiments, the hyaluronidase inhibitor-containing compositions of the present invention (which, within certain embodiments comprise one or more hyaluronidase inhibitor compound and a polymeric carrier) are fashioned in a manner appropriate to the intended use. Within certain aspects of the present invention, the composition should be biocompatible, and release one or more hyaluronidase inhibitor compounds over a period of several days to months. For example, in one aspect of the invention, “quick release” or “burst” hyaluronidase inhibitor-containing compositions are provided that release greater than 10%, 20%, or 25% of a hyaluronidase inhibitor compound over a period of 7 to 10 days. Such “quick release” compositions should, within certain embodiments, be capable of releasing hyaluronidase-inhibiting levels of a desired hyaluronidase inhibitor compound. Within other embodiments, “low release” hyaluronidase inhibitor-containing compositions are provided that release less than 5% (w/v) of a hyaluronidase inhibitor compound over a period of 7 to 10 days. Further, hyaluronidase inhibitor-containing compositions of the present invention should preferably be stable for several months and capable of being produced and maintained under sterile conditions.

Within certain aspects of the present disclosure, hyaluronidase inhibitor-containing compositions may be fashioned in any size ranging from about 0.050 nm to about 500 μm, depending upon the particular use. For example, when used for the purpose of cosmetic tissue augmentation (as discussed below), it is generally preferable to fashion the hyaluronidase inhibitor-containing composition in microspheres or microparticles having an average diameter of between about 0.1 to about 100 μm, preferably between about 0.5 and about 50 μm, and most preferably, between about 1 and about 25 μm. Alternatively, such compositions may also be applied as a solution in which the hyaluronidase inhibitor compound is solubilized in a micelle. The composition of the micelles may be polymeric in nature. For example, polymeric micelles may include a copolymer of MePEG and poly(D,L-lactide). Alternatively, such compositions may also be applied as a solution in which the HI is encapsulated in a liposome (see above). In certain other aspects, the HI is not encapsulated (e.g., contained) in a liposome. Alternatively, such compositions may also be applied as a solution in which the hyaluronidase inhibitor compound is encapsulated (e.g., contained) in the oil phase of an emulsion or microemulsion.

In one aspect, HA may be combined with a secondary carrier, which may be a polymer or non-polymer, that comprises one or more HI's. The secondary carrier may take a variety of forms and may provide for sustained and controlled release of the HI from the composition.

In one aspect, the secondary carrier is in the form of microparticles. “Microparticle,” as used herein refers, to one or a plurality of discrete solid particles which have a regular or irregular shape. Microparticles generally have a diameter (i.e., the distance spanning the widest point, or points, of the microparticle) of not more than about 500 μm. Nanoparticles typically have a diameter of less than about 500 nm.

Microparticles may be made from a variety of polymers, which may be bioresorbable or non-bioresorbable. “Bioresorbable” as used herein refers to the property of a composition or material being able to be cleared from a body after administration to a human or animal. Bioresorption may occur by one or more of a variety of means, such as, for example, dissolution, oxidative degradation, hydrolytic degradation, enzymatic degradation, metabolism, clearance of a component, its breakdown product, or its metabolite through routes such as, for example, the kidney, intestinal tract, lung or skin. Degradative mechanisms for bioresorption are collectively termed “biodegradation”.

In another aspect, the microparticles are in the form of microspheres. “Microsphere” as used herein refers to a microparticle that is essentially spherical in shape. Microspheres may be spherical, elliptoid or have a shape which approximates such a spherical or elliptoid shape, and may be smooth or have disruptions such as cracks or dimples. Microspheres typically have a mean diameter between about 500 nm and about 500 μm. In certain embodiments, the microparticles have a preferred average diameter of at least about 200 nm or 500 nm, 1 μm, 5 μm, 10 μm, 20 μm, 50 μm or 100 μm, 150 μm, 250 μm, 500 μm, 1000 μm, 2500 μm or 5000 μm, the optimal size being determined by the desired drug release properties and the application. In certain embodiments, the microparticles have a preferred average diameter of not more than about 200 nm or 500 nm, 1 μm, 5 μm, 10 μm, 20 μm, 50 μm or 100 μm, 150 μm, 250 μm, 500 μm, 1000 μm, 2500 μm or 5000 μm, the optimal size being determined by the desired drug release properties and the application.

In certain embodiments, microparticles are formed from one or more types of synthetic polymers. The synthetic polymer may be a polyester, which includes the residues of one or more of the monomers selected from lactide, lactic acid, glycolide, glycolic acid, ε-caprolactone, γ-caprolactone, hydroxyvaleric acid, hydroxybutyric acid, O-butyrolactone, γ-butyrolactone, gamma-valerolactone, γ-decanolactone, δ-decanolactone, trimethylene carbonate, 1,4-dioxane-2-one and 1,5-dioxepan-2one. The polyester may further include a residue having a chemical formula [—OC₆H₄COOH]. The polyester may include, poly(L-lactide) (PLLA), or poly(DL-lactide) (PDLLA), or poly(glycolide), or poly(DL-lactide-co-glycolide) (PLGA), poly(ε-caprolactone), poly(6-decanolactone), poly(6-valerolactone), or poly(lactic acid) (PLA). In other aspects, the polymer may include a polyether, such as a polyether that includes a residue of polyethylene glycol (PEG) or a copolymer thereof (e.g., PLA-block-PEC; or PLGA-block-PEG, or polypropylene oxide-block-PEG). In other aspects, the polymer may include a biologically derived polymer, such as, for example, a polysaccharide (e.g., chitosan, cellulose, alginate, or a derivative thereof).

The microparticles may be made from degradable synthetic polymers. Degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, P-butyrolactone, γ-butyrolactone, γ-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X (where X in a polyalkylene oxide (e.g, poly(ethylene glycol, poly(propylene glycol) and block copolymers of poly(ethylene oxide) and poly(propylene oxide) (e.g., PLURONIC and PLURONIC R series of polymers) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, O-butyrolactone, γ-butyrolactone, γ-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

In other aspects, the polymer used to prepare a microparticle may be non-biodegradable, such as poly(methylmethacrylate), poly(styrene), or poly(divinylbenzene).

The HI may be present in the microparticle at a concentration ranging from about 0.0001% to greater than 90% (weight of drug/weight of microparticle), depending on the type of HI and the type of polymers used to prepare the microparticle. In separate aspects, the HI may be present in the microparticle at a concentration of 0.0001% to 0.001%, 0.001% to 0.01%, or 0.01% to 0.1%, or 0.1% to 0.1%, or 1% to 10%, or 10% to 25%, or 25% to 50%, or 50% to 75%, or 75% to 85%, or greater than 90% (weight of drug/weight of microparticle).

Microparticles within the scope of this invention may have a wide range of release characteristics depending on the composition and the particular use. Microparticles may be prepared to provide sustained release of an HI over a period of several hours (e.g., 1 hour, 2 hours, 4 hours, 8 hours, 12 hours or 24 hours) to days (e.g., 1 day, 2 days, 3 days, 7 days, or 14 days) to months (e.g., 1 month, 2 months, 3 months, 6 months or 12 months) to years (e.g., up to 1 year, 2 years, 3 years). Release profiles may be characterized in terms of the initial rate, time for 50%, 90% or 100% drug release, or by appropriate kinetic models such as zero-order, first order, diffusion controlled (e.g., square-root of time, Higuchi model) kinetics, or by the number of distinct phases of release rate (e.g., monophasic, biphasic, or triphasic). The release profile may be characterized by the extent of its burst (initial) phase. The burst phase may result in little or large amounts of drug release and consequently microparticles may be defined as “low” or “high” burst systems. For example, low burst systems may release as little as about 30, 20,10 or even 5 or 1% of the total amount loaded in the initial phase of release. High burst systems may release at least about 50, 60, 70 or even 100% of the total amount of drug in the burst phase. The duration of the burst phase is dependant on the overall intended duration of the release profile. For microparticles intended to release all of the loaded drug within hours, the burst phase may occur over several minutes (e.g., 1 to 30 minutes). For microparticles intended to release over several days, the burst phase may on the order of hours (e.g., 1 to 24 hours). For microparticles intended to release over several weeks, the burst phase may be from several hours to several days (e.g., 12 hours to 7 days). An exemplary release profile describing a microparticles release characteristics may be a low burst microsphere, releasing less than 10% in the first 24 hours, followed by a phase of approximately zero-order release and a gradual reduction in rate after 5 days, until all of the drug is depleted.

A variety of methods are known in the art for preparing microparticles. Commonly used methods, which may be adapted to incorporate an HI into a microparticle include: (a) phase separation followed by solvent evaporation in dispersions such as o/o, w/o, o/w or w/o/w (o=oil, w=water), (b) use of super critical fluids (c) coacervation, (d) melt dispersions, (e) spray drying, (f) spray congealing, or (g) suspension coating. Representative examples of methods for preparing microparticles are disclosed in, e.g., U.S. Pat. Nos. 4,652,441; 5,100,669; 4,438,253 and 5,665,428.

In certain embodiments, the microparticles may be subjected to a process of lyophilization, comprising lyophilization of the liquid microparticle-containing composition to create a lyophilized powder. The powder may be combined directly with the hyaluronic acid. Alternatively, the lyophilized powder may be reconstituted with water or other aqueous media prior to combination with the hyaluronic acid.

The hyaluronidase inhibitor-containing compositions of the present invention may also be prepared in a variety of “paste” or gel forms. For example, within one embodiment of the invention, HI compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.).

Methods for incorporating hyaluronidase inhibitor compounds into a polymeric carrier are described in more detail below in the Examples.

The compositions of the present invention may, in addition to containing hyaluronic acid and a hyaluronidase inhibitor compound, also contain a bioactive hydrophobic compound. In one aspect, the composition contains hyaluronic acid, a hyaluronidase inhibitor compound, a carrier (polymer or non-polymer), and a bioactive hydrophobic compound. Within further aspects of the present invention, carriers are provided which are adapted to contain and release a hydrophobic compound. In certain embodiments, the carrier is a polymer. The carrier containing the hydrophobic compound may optionally be in combination with a carbohydrate, protein or polypeptide. Within certain embodiments, the carrier contains or comprises regions, pockets, or granules containing one or more hydrophobic compounds. For example, within one embodiment of the invention, hydrophobic compounds may be incorporated within a matrix that contains the hydrophobic compound, followed by incorporation of the matrix within a polymeric carrier. A variety of matrices can be utilized in this regard, including for example, carbohydrates and polysaccharides such as starch, cellulose, dextran, methylcellulose, and hyaluronic acid, proteins or polypeptides such as albumin, hyaluronic acid and gelatin. Within alternative embodiments, hydrophobic compounds may be contained within a hydrophobic core, and this core contained within a hydrophilic shell. For example, as described below in the Examples, indomethacin may be incorporated into a hydrophobic core (e.g., of the poly D,L lactic acid-PEG or MePEG aggregate) which has a hydrophilic shell.

Within certain aspects of the present invention, hyaluronidase inhibitor-containing compositions may be fashioned in such a manner that the hyaluronidase inhibitor compound is covalently attached to the hyaluronic acid used in the specific application. The hyaluronidase inhibitor compound can be attached directly to the hyaluronic acid or through a linker molecule (e.g., poly(ethylene glycol)). Once the conjugate is introduced or applied to the desired site, the hyaluronidase inhibitor compound may inhibit hyaluronidase while still attached to the hyaluronic acid.

The hyaluronidase inhibitor may be present in the composition in an amount effective to inhibit the degradation of hyaluronic acid by a hyaluronidase. The amount of HI in the composition depends on the type and potency of HI and the type and loction of the HA implant, the desired dose, as well as various other factors. For example, the HI may be present in the composition (e.g., a composition in a fluid or semi-solid form) in separate embodiments at a concentration of about 0.1 mg/ml or less, or about 0.1 mg/ml to 0.25 mg/ml, or about 0.25 mg/ml to 0.5 mg/ml, or about 0.5 mg/ml to 1 mg/ml, or about 1 mg/ml to 5 mg/ml, or about 5 mg/ml to 10 mg/ml, or about 10 mg/ml to 25 mg/ml, or about 25 mg/ml to 100 mg/ml, or about 100 mg/ml to 250 mg/ml, or about 250 mg/ml to 350 mg/ml, or about 350 mg/ml to 500 mg/ml.

For certain HI's (e.g., compounds having a defined MW) the amount of HI present in the composition may be expressed in terms of molarity. For example, the HI may be present in the composition in separate embodiments at a concentration of about 1 mM or less (e.g., about 1 μm to 10 μm, or about 10 μm to 100 μm, or about 100 μm to 1 mM), or about 1 mM to 2.5 mM, or about 2.5 mM to 5 mM, or about 5 mM to 10 mM, or about 10 mM to about 25 mM, or about 25 mM to 50 mM, or about 50 mM to 100 mM, or about 100 mM to 250 mM, or about 250 mM to about 350 mM. In separate embodiments, the HI may be present in the composition at a concentration of less than about 50% (by weight), or less than about 25%, or less than about 10%, or less than about 5%, or less than about 2%, or less than about 1%, or less than about 0.5%, or less than about 0.25%, or less than about 0.1%., or less than about 0.01%, or less than about 0.001%, or less than about 0.0001%.

For certain types of HA compositions (e.g., materials in the form of films and meshes), the concentration of HI may be expressed in terms of area. For example, in separate embodiments, the composition may include about about 0.0001 mg to about 0.001 mg per square inch of material, or 0.001 mg to about 0.01 mg per square inch of material, 0.01 mg to about 0.1 mg per square inch of material, or about 0.1 mg to about 1 mg per square inch of material, or about 1 mg to about 5 mg per square inch of material, or about 5 mg to about 10 mg per square inch of material, or about 10 mg to about 20 mg per square inch of material, or about 20 mg to about 50 mg per square inch of material, or about 50 mg to about 100 mg per square inch of material, about 100 mg to about 250 mg per square inch of material.

The total dose of HI delivered from the HA implant may be, in separate embodiments, less than about 0.1 mg, or about 0.1 mg to 0.5 mg, or about 0.5 mg to 1 mg, or about 1 mg to 5 mg, or about 5 mg to 10 mg, or about 10 mg to 20 mg, or about 20 mg to 100 mg, or about 100 mg to 200 mg, or about 200 mg to 350 mg, or about 350 mg to 500 mg. The total dose may be expressed in terms of amount of HI delivered per volume of aqueous body fluid. For example, in separate embodiments, the total dose of HI delivered from an HA implant may range from about 0.01 mg/ml to 0.1 mg/ml, or about 0.1 mg/ml to 1 mg/ml, or about 1 mg/ml to 10 mg/ml, or about 10 mg/ml to 25 mg/ml, or about 25 mg/ml to about 100 mg/ml of aqueous body fluid.

Within certain embodiments of the invention, the compositions provided herein may be further modified in order to enhance their utility. For example, within one embodiment, compounds or factors which aid clotting (e.g., thrombin) may be added to the compositions described herein. The HA-HI composition may further include a neurotoxin such as a botulinum toxin, which is commercially available under the trade name BOTOX from Allergan, Inc. (Irvine, Calif.) and/or an anesthetic such as lidocaine, benzocaine or prilocalne. The anesthetic can further comprise a polymeric carrier, as described above, which can be used to assist the formulation of the anesthetic into the HA-HI composition and/or to modulate the release of the anesthetic from the HA-HI composition. Therapeutic agents, such as antibiotics, anti-infective agents (e.g., 5-fluorouracil), anti-inflammatory agents (e.g., steroidal and non-steroidal), pain relieving agents, anti-scarring (anti-fibrotic) agents, scarring (fibrotic) agents, may be added to the present compositions to provide additional therapeutic benefits.

In addition, the compositions may further include an additive. Representative examples of additives include solvents, antioxidants (e.g., sulfites and ascorbic acid), binders, pore formers, preservatives (e.g., paraoxybenzoic acid esters, chlorobutanol, benzylalcohol, phenethyl alcohol, dehydroacetic acid, sorbic acid, etc), bacteriostatic agents (e.g., bismuth tribromophenate, methyl hydroxybenzoate, bacitracin, ethyl hydroxybenzoate, propyl hydroxybenzoate, erythromycin, chlorocresol, benzalkonium chlorides, and the like), and bactericidal (also known as bacteriacidal) agents. A dye or other coloring agent may be added to enhance visualization of the composition. The dye or coloring agent may be either permanent or transient (e.g., methylene blue). Representative examples of dyes include these suitable for food such as those known as F. D. and C. dyes, and natural coloring agents such as grape skin extract, beet red powder, beta carotene, annato, carmine, turmeric, paprika, and the like). Other examples of agents to improve visualization of the present compositions in a clinical setting include radio-opaque or X-ray opaque materials, such as tantalum, and MRI contrast agents.

Any of the compositions described herein may be provided in a sterile form. Sterilization in this embodiment may be accomplished by a number of means accepted in the industry and listed in the USP XXII <1211>, including without limitation autoclaving, dry heat, gas sterilization, and filtration. Preferably, sterilization should be accomplished by a method that does not break down the HA or HI. Typically, sterilization is achieved by a method other than irradiation as HA tends to decompose upon exposure to y radiation. Sterilization may be maintained by what is termed aseptic processing, defined also in USP XXII <1211>. Acceptable gases used for gas sterilization include ethylene oxide. Filtration may be accomplished using a filter with suitable pore size, such as 0.22 μm, and of a suitable material, such as TEFLON. Furthermore, a sterile composition may be achieved by using a combination of these sterilization methods and optionally aseptic techniques. In certain aspects of the invention comprising microparticles greater than 200 nm in diameter, a method of sterilization other than filtration should be used since the particles would not pass easily through a 0.22 μm filter. Since not all components of certain embodiments of the invention may be conveniently sterilized by a single method, sterilization may be accomplished by sterilizing components of the embodied invention in separate steps and combining the sterilized components into the embodied composition.

IV. Clinical Application

1. HI-Loaded Hyaluronic Acid Orthopedic Implants

A variety of hyaluronic acid implants have been developed for use in orthopedic surgery as tissue filler and to serve as a scaffold for healing and repair. Hyaluronic acid, which is an important organic component of connective tissue and of cartilage, can be combined with mineral formulations, autogenous bone marrow, bone graft, and/or growth factors (such as fibroblast growth factor (FGF) or bone morphogenic proteins (BMPs)) for use as a tissue substitute or a skeletal repair product. Typical applications include, but are not restricted to, total joint replacement surgery (e.g., artificial hips, knees, etc.), spinal fusion surgery, long bone fractures, repair of traumatic bone defects, voids, or gaps, to augment an autograph, and as a bone filler at bone graft harvesting sites. For example, OSSIGEL is a viscous formulation of hyaluronic acid (HA) and basic fibroblast growth factor (bFGF) designed to accelerate bone fracture healing (Orquest, Inc.).

Representative examples of hyaluronic acid compositions used in orthopedic procedures are described in U.S. Pat. Nos. 6,764,517; 6,514,514; 6,730,129; and 6,652,887.

In the present invention, an inhibitor of hyaluronidase is added to the hyaluronic acid-containing implant or composition, alone or in a sustained-release form, to decrease the rate of degradation of the HA and prolong the composition/implant's activity in vivo beyond that seen with HA alone (e.g., consistently longer than 2 weeks in >75% of patients and longer than 2 months in >25% of patients). In one aspect, the composition may be delivered at a desired location such at the site of a fracture or void in a bone to augment the bone or replace lost bone. The total dose of HI delivered, the rate of dose release, and the duration of drug release from the HA can be tailored to significantly prolong the activity of the hyaluronic acid implant as required. As described above, in some embodiments the HI-HA composition can be further combined with mineral formulations, autogenous bone marrow, bone graft material, and/or growth factors (such as fibroblast growth factor (FGF), transforming growth factor (TGF), platelet-derived growth factor (PDGF) or bone morphogenic proteins (BMPs)) for use as a tissue substitute or a skeletal repair product. The following compositions are ideally suited for use in this indication.

The HI may be combined with a polymer system to provide sustained release of the agent. The material suitable for delivery of a HI agent in combination with HA in orthopedic applications can be composed of a non-degradable or a degradable material. Suitable degradable materials include, but are not limited to, resorbable ceramics composed of β-tricalcium phosphate (e.g., VITOSS made by Orthovita, Inc., PROOSTEON 500R made by E-Interpore-Cross International), hydroxyapatite or Ca₁₀(PO₄)₆OH (e.g., BIOOSS made by Geistlich Biomaterials Inc., OSTEOGRAF made by Ceremed Denta Inc.), calcium carbonate or CaCO₃, calcium sulphate (e.g., OSTEOSET and ALLOMATRIX made by Wright Medical), calcium phosphate (e.g., CALCIBON made by Merck, NORIAN SRS), crosslinked materials of PEG, gelatin, collagen, GELFOAM, demineralized bone matrix, bone allografts (e.g., ALLOGRO, ORTHOBLAST, OPTEFORM, GRAFTON), polysaccharides, carbohydrates, proteins (e.g., albumin, casein, whey proteins, plant proteins, fish proteins etc), autologous bone, demineralized bone matrix, alginates, starch, cellulose derivatives (HPC etc), cellulose, cellulose esters, blends and copolymers thereof, chitosan, chitosan derivatives, polyester-polyalkylene oxide block copolymers (e.g., PLGA-PEG-PLGA, MePEG-PLGA, etc), degradable polyesters, polyanhydrides, polyorthoesters, polyphosphoesters, polyphosphazines, and cyanoacrylate polymers. Particularly useful degradable polymers for use in the practice of this invention include injectable PEG-containing formulations such as COSEAL (Angiotech Biomaterials Corp., Palo Alto, Calif.), FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL; and other low molecular weight polymers that can be excreted.

Suitable non-degradable materials for delivery of an HI in combination with HA for orthopedic applications include crosslinked compositions that comprise PVA, PVP, polyacrylamide, methyl methacrylate (MMA) and methyl methacrylate styrene (MMA-styrene) which when mixed together form polymethyl methacrylate (PMMA) or bone cement (e.g., SIMPLEX P made by Stryker Howmedica, ZIMMER REGULAR and ZIMMER LOW VISCOSITY CEMENT made by Zimmer, PALACOS made by Smith and Nephew, CMW-1 and CMW-2 made by Wright Medical, DEPUY ENDURANCE made by DePuy), synthetic cancellous bone void fillers (e.g., CORTOSS, Orthovita), pHEMA, poly(vinyl PEG), poly(styrene sulfonate), poly(acrylic acid), poly(methacrylic acid), as well as other polymers that are known in the literature to form hydrogels. Additional compositions include blends and copolymers of the agents listed above. Calcium phosphate such as basic calcium phosphate and hydroxyapatite may be used in combination with the hyaluronidase inhibitors.

It should be apparent to one of skill in the art that potentially any hyaluronidase inhibitor may be utilized alone, or in combination, in the practice of this embodiment as described above. Exemplary HI agents for use in combination with HA orthopedic procedures include sulphated polysaccharides; pharmaceutical excipients; diblock copolymers and indomethacin or gold compounds; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate. For example, many problems associated with effective implantation of orthopedic implants involve an unwanted inflammatory response. Therefore, since both indomethacin and gold compounds have anti-inflammatory properties, these compounds may serve a dual purpose in these applications.

All these compounds may be used alone, or further combined with bone morphogenic proteins and/or osteogenic growth factors (such as transforming growth factor, platelet-derived growth factor, fibroblast growth factor) as well as analogues and derivatives of the aforementioned. Examples of bone morphogenic protein include, e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242(4885); 1528-1534.

Suitable doses of these compounds may be such as to provide a steady concentration of each agent to elicit a prolonged inhibitory effect on hyaluronidase. These concentrations are approximate and may be adjusted depending on the potency of the compound and duration of effect required: aurothiomalate 10 mM and other gold compounds, indomethacin 1 mg/ml, heparin 1 mg/ml, sulphated polysaccharides 2 mg/ml, and propylene glycol, TRITON X-100, PEG, SPAN, PLURONIC L101 and carboxymethyl cellulose all at 10 mg/ml. In order to attain this concentration, a dose of approximately 10 times that required dose per ml may be needed (e.g., 10 mg indomethacin, 20 mg of sulphated polysaccharides, 100 mg of propylene glycol, TRITON X-100, PEG, SPAN, PLURONIC or carboxymethyl cellulose) in a volume that may be exposed to several ml of aqueous body fluid. So for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application as well as duration of effect required. At sites with a higher fluid turn over, more hyaluronidase inhibitor may be given. Furthermore, if the inhibitor were released in a controlled manner from a polymeric dosage form then the applied total dose may be calculated by one skilled in the art based on inhibitor release profiles, site of application, turn over of body fluid in that area and other parameters such as age and general health.

2. HI-Loaded Hyaluronic Acid Spinal Surgery Implants

Back pain is the number one cause of healthcare expenditures in the United States and accounts for over $50 billion in costs annually ($100 billion worldwide). Over 12 million people in the U.S. have some form of degenerative disc disease (DDD) and 10% of them (1.2 million) will require surgery to correct their problem.

In healthy individuals, the vertebral column is composed of vertebral bone plates separated by intervertebral discs that form strong joints and absorb spinal compression during movement. The intervertebral disc is comprised of an inner gel-like substance called the nucleus pulposus which is surrounded by a tough fibrocartilagenous capsule called the annulus fibrosis. The nucleus pulposus is composed of a loose framework of collagen fibrils and connective tissue cells (resembling fibroblasts and chondrocytes) embedded in a gelatinous matrix of glycosaminoglycans and water. The annulus fibrosus is composed of numerous concentric rings of fibrocartilage that anchor into the vertebral bodies. The most common cause of DDD occurs when tears in the annulus fibrosis create an area of localized weakness that allow bulging, herniation or sequestration of the nucleus pulposis and annulus fibrosis into the spinal canal and/or spinal foramena. The bulging or herniated disc often compresses nerve tissue such as spinal cord fibers or spinal cord nerve root fibers. Pressure on the spinal cord or nerve roots from the damaged intervertebral disc results in neuronal dysfunction (numbness, weakness, tingling), crippling pain, bowel or bladder disturbances and can frequently cause long-term disability. Although many cases of DDD will spontaneously resolve, a significant number of patients will require surgical intervention in the form of minimally invasive procedures, microdiscectomy, major surgical resection of the disc, spinal fusion (fusion of adjacent vertebral bone plates using various techniques and devices), and/or implantation of an artificial disc.

Open surgery to relieve pressure on a spinal nerve typically involves resection of a ruptured lumbar disc (and portions of the bone surrounding a spinal nerve root—known as laminectomy). The patient is placed in a modified kneeling position under general anesthesia. An incision is made in the posterior midline and the tissue is dissected away to expose the appropriate interspace; the ligamentum flavum is dissected and in some cases portions of the bony lamina are removed to allow adequate visualization. The nerve root is carefully retracted away to expose the herniated fragment and the defect in the annulus. Typically, the cavity of the disc is entered from the tear in the annulus and the loose fragments of the nucleus pulposus are removed with pituitary forceps. Any additional fragments of disc sequestered inside or outside of the disc space are also carefully removed and the disc space is forcefully irrigated to remove to remove any residual fragments. If tears are present in the dura, the dura is closed with sutures that are often augmented with fibrin glue. The tissue is then closed with absorbable sutures.

Microlumbar disc excision (microdiscectomy) can be performed as an outpatient procedure and has largely replaced laminectomy as the intervention of choice for herniated discs. A one inch incision is made from the spinous process above the disc affected to the spinous process below. Using an operating microscope, the tissue is dissected down to the ligamentum flavum and bone is removed from the lamina until the nerve root can be clearly identified. The nerve root is carefully retracted and the tears in the annulus are visualized under magnification. Microdisc forceps are used to remove disc fragments through the annular tear and any sequestered disc fragments are also removed. As with laminectomy, the disc space is irrigated to remove any disc fragments, any dural tears are repaired and the tissue is closed with absorbable sutures. It should be noted that anterior (abdominal) approaches can also be used for both open and endoscopic lumbar disc excision. Cervical and thoracic disc excisions are similar to lumbar procedures and can also be performed from a posterior approach (with laminectomy) or as an anterior discectomy with fusion.

Unfortunately, in a significant number of patients, post-surgical scarring in the tissues surrounding the nerve root exerts pressure on the nerve, causes irritation, and leads to a recurrence of pain and other neurological symptoms. To reduce the incidence of this complication, many surgeons infiltrate the area surrounding the nerve with implants composed of hyaluronic acid. The HA prevents adjacent tissues from coming into contact with the nerve and scar tissue from forming on, and ultimately constricting around, the spinal nerve. However, the HA is quickly absorbed by the body over a period of several days—often before healing is complete and allowing the scar tissue to form around the nerve.

In the present invention, an inhibitor of hyaluronidase is added to the hyaluronic acid-containing implant or composition, alone or in a sustained-release form, to decrease the rate of degradation of the HA and prolong the composition/implant's activity in vivo beyond that seen with HA alone (e.g., consistently longer than 2 weeks in >75% of patients and longer than 2 months in >25% of patients). The total dose of HI delivered, the rate of dose release, and the duration of drug release from the HA can be tailored to significantly prolong the activity of the hyaluronic acid implant as required. This would allow the barrier to function longer in vivo and reduce the likelihood of scar tissue from forming around the nerve root. An HI-HA implant may reduce the incidence of spinal surgery failure, prevent the recurrence of pain and neurological symptoms, and reduce the need to perform repeat surgical interventions to remove scar tissue.

Examples of suitable commercial HA products that may be combined with an HI for use in spinal surgery include: RESTYLANE, HYLAFORM, PERLANE, SYNVISC, SEPRAFILM, SEPRACOAT, INTERGEL, and LUBRICOAT.

Representative examples of hyaluronic acid compositions used in spinal surgery procedures are described in U.S. Pat. Nos. 6,719,797; 5,258,043; and 4,904,260.

The HI may also be combined with a polymer system to provide sustained release of the agent. The material suitable for delivery of a HI agent for the purposes of this invention can be composed of a non-degradable or a degradable material; however, a degradable material is preferred. Suitable degradable materials include, but are not limited to, crosslinked materials of PEG, gelatin, collagen, GELFOAM, bone allografts (e.g., ALLOGRO, ORTHOBLAST, OPTEFORM, GRAFTON), polysaccharides, carbohydrates, proteins (e.g., albumin, casein, whey proteins, plant proteins, fish proteins etc), autologous bone, demineralized bone matrix, alginates, starch, cellulose derivatives (HPC etc), cellulose, cellulose esters, blends and copolymers thereof, chitosan, chitosan derivatives, polyester-polyalkylene oxide block copolymers (e.g., PLGA-PEG-PLGA, MePEG-PLGA, etc), degradable polyesters, polyanhydrides, polyorthoesters, polyphosphoesters, polyphosphazines, cyanoacrylate polymers, injectable PEG-containing formulations such as COSEAL, FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL; and other low molecular weight polymers that can be excreted.

It should be apparent to one of skill in the art that potentially any hyaluronidase inhibitor may be utilized alone, or in combination, in the practice of this embodiment as described above. Exemplary HI agents for use in combination with HA in spinal surgery procedures include: heparin, aurothiomalate, dextran sulphate, fucoidan, propylene glycol; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate; as well as analogues and derivatives of the aforementioned.

Suitable doses of these compounds may be such as to provide a steady concentration of each agent to elicit a prolonged inhibitory effect on hyaluronidase. These concentrations are approximate and can be adjusted according to the potency of the compound, the duration of effect and the anticipated rate of breakdown of the hyaluronic acid dependent on the anatomical location: aurothiomalate 10 mM, indomethacin 1 mg/ml, heparin 1 mg/ml, sulphated polysaccharides 2 mg/ml, and propylene glycol, TRITON X-100, PEG, SPAN, PLURONIC L101 and carboxymethyl cellulose all at 10 mg/ml. In order to attain this concentration a dose of approximately 10 times that required dose per ml may be needed (e.g., a total weight of 10 mg indomethacin, 20 mg sulphated polysaccharides, 100 mg propylene glycol, TRITON, PEG, SPAN, PLURONIC or carboxymethyl cellulose) in a volume that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health.

3. HI-Loaded Hyaluronic Acid Surgical Adhesion Barriers

Surgical adhesion formation is a complex process in which bodily tissues that are normally separate grow or scar together. Adhesions are connections or bridges of scar tissue that occur between adjacent tissues that are damaged during surgery. Surgical trauma, as a result of tissue drying, ischemia, thermal injury, infection or the presence of a foreign body, has long been recognized as a stimulus for tissue adhesion formation. Mechanical injuries include crushing of the bowel (Choate et al., Arch. Surg. 88:249-254, 1964) and stripping or scrubbing away the outer layers of bowel wall (Gustavsson et al., Acta Chir. Scand. 109:327-333, 1955). Dividing major vessels to loops of the intestine induces ischemia (James et al., J. Path. Bact. 90:279-287, 1965) that can lead to adhesions. Foreign material that may be introduced into the area and cause adhesions includes talcum (Green et al., Proc. Soc. Exp. Biol. Med. 133:544-550, 1970), gauze sponges (Lehman and Boys, Ann. Surg 111:427-435, 1940), toxic chemicals (Chancy, Arch. Surg. 60:1151-1153, 1950), bacteria (Moin et al., Am. J. Med. Sci. 250:675-679, 1965) and feces (Jackson, Surgery 44:507-518, 1958). As a result, surgical adhesions are a major cause of failed surgical therapy and are the leading cause of bowel obstruction and infertility. Other adhesion-related complications include chronic pelvic pain, urethral obstruction and voiding dysfunction. It is estimated that post-operative adhesions occur in 60% to 90% of patients undergoing major gynecological or abdominal surgery and the estimated annual cost of treating abdominal adhesions is thought to exceed $2 billion.

Generally, adhesion formation is an inflammatory reaction in which factors are released, increasing vascular permeability and resulting in fibrinogen influx and fibrin deposition. Fibrin deposition forms a matrix that bridges abutting tissues or organs damaged by surgery or disease (e.g., inflammatory bowel disease, Crohn's disease). Under normal circumstances, most fibrin matrices between organs degrade during the healing process. However, when fibrin matrices fail to degrade, fibroblasts accumulate, attach to the matrix, deposit collagen and induce angiogenesis. The result is the formation of permanent bands of fibrous scar tissue linking organs or tissues together that should normally remain separate. If this cascade of events can be prevented within 4 to 5 days following surgery, then adhesion formation can be reduced.

Hyaluronic acid-based products have been developed to prevent or reduce adhesions following a variety of surgical procedures. Hyaluronic acid (typically sodium hyaluronate) films, gels or sprays serve to form a temporary bioresorbable barrier separating adjacent tissues (i.e., the physical presence of the barrier between two tissues prevents them from coming into direct contact with each other and scarring together during the healing process). Unfortunately, approximately 24 to 48 hours after placement, the membrane becomes hydrated and starts to get resorbed. Although this is suitable for some surgical procedures, in other procedures (or in compromised patients such as diabetics who heal more slowly) this duration of activity is insufficient and the barrier is resorbed before healing is completed—placing the patient at increased risk for forming adhesions.

In the present invention, an inhibitor of hyaluronidase is added to the hyaluronic acid-containing surgical adhesion film, gel, or spray, either alone or in a sustained-release form, to decrease the rate of degradation of the HA and prolong the composition/implant's activity in vivo beyond that seen with HA alone (e.g., consistently longer than 2 days in >90% of patients and longer than 7 days in >50% of patients). In one aspect, the HI-containing formulation is delivered to a desired location, such as the surface of a target tissue or organ, during a surgical procedure to prevent the formation of an adhesion. Examples of such surfaces representing desired locations include, but are not limited to, the inner surfaces of the reproductive tract including fallopian tubes, ovaries, and uterus, within the digits surrounding tendons, the spine, the inner muscular surface of the intraperitoneal cavity and the outer surfaces of the digestive tract including as examples the small and large intestines, stomach and surfaces of accessory organs such as kidneys, spleen, and liver.

The total dose of HI delivered, the rate of dose release, and the duration of drug release from the HA can be tailored to significantly prolong the activity of the hyaluronic acid implant as required. This would allow the barrier to function longer in vivo and reduce the likelihood of scar tissue from forming between adjacent organs or tissues. An HI-HA implant may reduce the incidence of and/or the severity of adhesions that may form following abdominal and gynecological surgery. Adhesion reduction may prevent the occurrence of pain, bowel obstruction, and infertility, and reduce the need to perform repeat surgical interventions to remove scar tissue.

Utilizing the agents, compositions and methods provided herein a wide variety of surgical adhesions and complications of surgery can be treated or prevented. Adhesion formation or unwanted scar tissue accumulation/encapsulation complicates a variety of surgical procedures. As described above, surgical adhesions can potentially complicate virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule which compromises or impairs the function of the surgical implant (e.g., breast implant, artificial joint, surgical mesh, vascular graft, dural patch, the pericardial sac). Chronic inflammation and scarring also occurs during surgery to correct chronic sinusitis or removal of other regions of chronic inflammation (e.g., foreign bodies, infections (fungal, mycobacterium)). An HI-HA implant may be used in the management of these surgical conditions as well.

Examples of suitable commercial HA products that may be combined with an HI for use in abdominal, gastrointestinal, coronary, peripheral nerve and tendon, orthopedic, gynecological and other surgeries include: RESTYLANE, HYLAFORM, PERLANE, SYNVISC, and LUBRICOAT, INTERGEL from Lifecore Biomedical, and the SEPRAFILM, SEPRAGEL, and SEPRACOAT adhesion barriers from Genzyme Biosurgery, Inc. Other HA products include implants such as OSSIGEL cross-linked HA products from Anika Therapeutics, such as ATRISOL, the INCERT family of bioabsorbable, cross-linked hyaluronic acid (HA) products, and HA derivatives such as HYALGEL-R from Genzyme Biosurgery.

Representative examples of hyaluronic acid compositions used to prevent surgical adhesions are described in U.S. Pat. Nos. 6,723,709; 6,531,147; and 6,464,970.

The HI may be combined with a polymer system to provide sustained release of the agent. The material suitable for delivery of a HI agent for the purposes of this invention can be composed of a non-degradable or a degradable material; however, a degradable material is preferred. Suitable degradable materials include, but are not limited to, crosslinked materials of PEG, gelatin, collagen, GELFOAM, bone allografts (e.g., ALLOGRO, ORTHOBLAST, OPTEFORM, GRAFTON), polysaccharides, carbohydrates, proteins (e.g., albumin, casein, whey proteins, plant proteins, fish proteins etc), autologous bone, demineralized bone matrix, alginates, starch, cellulose derivatives (e.g., HPC), cellulose, cellulose esters, blends and copolymers thereof, chitosan, chitosan derivatives, polyester-polyalkylene oxide block copolymers (e.g., PLGA-PEG-PLGA, MePEG-PLGA, etc), degradable polyesters, polyanhydrides, polyorthoesters, polyphosphoesters, polyphosphazines, cyanoacrylate polymers, injectable PEG-containing formulations such as COSEAL, FOCALSEAL, SPRAYGEL, DURASEAL or a compositions containing a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL, and other low molecular weight polymers that can be excreted.

HI-HA containing surgical adhesion barriers may be used in a variety of surgical procedures including abdominal surgery, gynecologic and pelvic surgery, spinal surgery, cardiac surgery, tendon and peripheral nerve surgery, and sinus surgery. Preferred methods of administering the HI-HA composition include direct application to the mesenteric surface as a “gel”, “suspension”, “solution”, “paste”, “film”, or “wrap” (e.g., a film, mesh, or membrane that can be wrapped around all or a portion of a body passageway, organ, or tissue surface) at the time of surgery or with endoscopic, ultrasound, CT, MRI, or fluoroscopic guidance); “coating” the HA surgical implant with an HI composition; and placement of a HI-eluting polymeric implant at the surgical site. During endoscopic procedures, the HI-HA preparation may be applied as a “spray”, via delivery ports in the endoscope, to the mesentery of the abdominal and pelvic organs manipulated during the operation.

It should be apparent to one of skill in the art that potentially any hyaluronidase inhibitor may be utilized alone, or in combination, in the practice of this embodiment as described above. Exemplary HI agents for use in combination with HA in the prevention of surgical adhesions include aurothiomalate; dextran sulphate; fucoidan; propylene glycol; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate; as well as analogues and derivatives of the aforementioned.

Suitable doses of these compounds may be such as to provide a steady concentration of each agent to elicit a prolonged inhibitory effect on hyaluronidase. These concentrations are approximate and may be adjusted depending on the potency of the compound and duration of effect required: aurothiomalate 10 mM, indomethacin 1 mg/ml, heparin 1 mg/ml, dextran sulphate at 2 mg/ml and propylene glycol, TRITON X-100, PEG, SPAN, PLURONIC L101 and carboxymethyl cellulose all at 10 mg/ml. In order to attain this concentration a dose of approximately 10 times that required dose per ml may be needed (e.g., a total weight of 10 mg indomethacin, 20 mg of sulphated polysaccharides, 100 mg of propylene glycol, TRITON, PEG, SPAN, PLURONIC or carboxymethyl cellulose) in an area or volume that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health.

4. HI-Loaded Hyaluronic Acid Cosmetic Implants

A variety of injectable hyaluronic acid products have been developed for soft tissue augmentation to correct facial scars, diminish facial lines and augment the lips. Specifically, such implants are indicated for the treatment of a variety of contour deficiencies including (but not restricted to) correction of acne scars, atrophy from disease or trauma, glabellar frown lines, nasolabial folds, or defects secondary to rhinoplasty, skin graft or other surgery and other soft tissue defects. Manufactured synthetic hyaluronic gels commercially available for this purpose include RESTYLANE and PERLANE and HYLAFORM (also known as HYLAN B) from Genzyme Corporation. Other examples of commercial HA products that may be combined with an HI for use in cosmetic injections include: ACHYAL from Meiji Seika Kaisha, Ltd. (Japan), JUVEDERM from L.E.A. Derm (France), MACDERMOL from Laboratoires O.R. GE V. MacDermol (France), and ROFILAN Hylan Gel from Rofil Medical International (Holland).

Unfortunately, repeated “touch up” procedures are often required as the implant is colonized by host connective tissue cells and inflammatory cells which produce hyaluronidase and other enzymes capable of breaking down the HA implant over time. An injectable hyaluronic acid containing a hyaluronidase inhibitor (HI), both alone or in a sustained release preparation, can result in increased durability of the implant and reduce the number of subsequent repeat injections. Although any of the previously described hyaluronidase inhibitors may be suitable for incorporation into a dermal HA injection, the following are particularly preferred: aurothiomalate, indomethacin, propylene glycol, dextran sulphate, fucoidan, hederagenin, flavonoids, agents that modulate allergic reactions, phenolic compounds, and carboxymethyl cellulose.

Regardless of the formulation utilized, administration of the HI-loaded HA injection may proceed in the following manner. A pre-loaded syringe with a fine gauge needle (30 or 32 gauge) containing the HI-HA implant material is used. The patient is placed in a sitting position with the table back slightly reclined. Topical lidocaine and/or prilocalne can be used for anesthesia. The needle is inserted at an angle to the skin and advanced into the superficial dermal tissue. A sufficient amount of implant material is extruded to repair the soft tissue contour defect. In the case of HI-loaded RESTYLANE, overcorrection (injection of more material than is ultimately needed) is required as some of the injected material dissipates in the hours following injection. HI-loaded PERLANE is typically used to correct deeper lines and is injected deeper into the dermis.

Representative examples of hyaluronic acid compositions used in cosmetic surgery injections are described in U.S. Pat. Nos. 5,633,001; 5,256,140; and 6,703,041.

The HI may be combined with a polymer system to provide sustained release of the agent as part of an HA dermal injection. The material suitable for delivery of a HI agent for the purposes of this invention can be composed of a non-degradable or a degradable material; however, a degradable material is preferred. Suitable degradable materials include, but are not limited to, crosslinked materials of PEG, gelatin, collagen, GELFOAM, polysaccharides, carbohydrates, proteins (e.g., albumin, casein, whey proteins, plant proteins, fish proteins etc), alginates, starch, cellulose derivatives (HPC etc), cellulose, cellulose esters, blends and copolymers thereof, chitosan, chitosan derivatives, polyester-polyalkylene oxide block copolymers (e.g., PLGA-PEG-PLGA, MePEG-PLGA, etc), degradable polyesters, polyanhydrides, polyorthoesters, polyphosphoesters, polyphosphazines, cyanoacrylate polymers, injectable PEG-containing formulations such as COSEAL, FOCALSEAL, SPRAYGEL, DURASEAL or compositions comprising a pentaerythritol poly(ethylene glycol)ether tetra-sulfhydryl] (4-armed thiol PEG), pentaerythritol poly(ethylene glycol)ether tetra-succinimidyl glutarate] (4-armed NHS PEG) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL, and other low molecular weight polymers that can be excreted.

The HA-HI composition may further comprise an anesthetic such as lidocaine, benzocaine or prilocalne and/or a neurotoxin such as a botulinum toxin.

It should be apparent to one of skill in the art that potentially any hyaluronidase inhibitor may be utilized alone, or in combination, in the practice of this embodiment as described above. Exemplary HI agents for use in combination with HA in cosmetic injection procedures include aurothiomalate, indomethacin, propylene glycol, carboxymethyl cellulose, dextran sulphate, fucoidan and heparin, as well as analogues and derivatives of the aforementioned.

Suitable doses of these compounds may be such as to provide a steady concentration of each agent to elicit a prolonged inhibitory effect on hyaluronidase. These concentrations are approximate and may be adjusted depending on the potency of the compound and duration of effect required: aurothiomalate 10 mM, indomethacin 1 mg/ml, heparin 1 mg/ml, sulphated polysaccharides 2 mg/ml, and propylene glycol, TRITON X-100, PEG, SPAN, PLURONIC L101, and carboxymethyl cellulose all at 10 mg/ml. In order to attain this concentration a dose of approximately 10 times that required dose per ml may be needed (e.g., a total weight of 10 mg indomethacin, 20 mg of sulphated polysaccharides, 100 mg of propylene glycol, TRITON, PEG, SPAN, PLURONIC or carboxymethyl cellulose) in an area that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health.

5. HI-Loaded HA Ocular Implants

Viscoelastic solutions of HA have been used to act as a tissue lubricant and also to maintain the volume of the eye fluid during surgery on the inside of the eye (e.g., as a vitreous substitute during cataract extraction surgery, intraocular lens implantation, retinal reattachment, phacoemulsification surgery, corneal transplantation, and glaucoma filtering surgery). AMVISC, AMVISC PLUS and OCUCOAT (Bausch & Lomb) are high molecular weight, viscoelastic and injectable HA solutions used to maintain eye shape and protect delicate tissues during cataract removal, corneal transplant and glaucoma surgery. HA-based ophthalmic viscoelastic products include PROVIS, VISCOAT, DUOVISC, and CELLUGEL from Alcon Laboratories; HEALON, HEALON G, and HEALON 5 from Pharmacia & Upjohn, VITRAX from Allergan; BIOLON from Bio-Technology General; STAARVISC from Anika Therapeutics/Staar Surgical; SHELLGEL from Anika Therapeutics/Cytosol Opthalmics; and UNIVISC from Novartis.

Representative examples of hyaluronic acid compositions used in ocular surgery are described in U.S. Pat. Nos. 5,728,405; 6,635,267; 6,465,588; and 6,242,480, 6,620,927; 5,728,405; 6,635,267; 6,465,588; and 6,242,480.

According to the present invention, differential loading of an HI into a HA ocular implant may be used for accurately controlling the dissolution rate of the ocular implant. In the present invention, an HI is added to the HA-containing implant or composition in a sustained-release form to decrease the rate of degradation of the hyaluronic acid and prolong the composition/implant's activity in vivo beyond that seen with HA alone (e.g., consistently longer than 1 month in >75% of patients). The total dose delivered, the rate of dose release, and the duration of drug release from the matrix can be tailored to significantly prolong the activity of the collagen implant as required.

The HI may be combined with a polymer system to provide sustained release of the agent as part of an HA dermal injection. The material suitable for delivery of a HI agent for the purposes of this invention can be composed of a non-degradable or a degradable material; however, a degradable material is preferred. Suitable degradable materials include, but are not limited to, crosslinked materials of PEG; gelatin, collagen, GELFOAM, polysaccharides, carbohydrates, proteins (e.g., albumin, casein, whey proteins, plant proteins, fish proteins etc), alginates, starch, cellulose derivatives (HPC etc), cellulose, cellulose esters, blends and copolymers thereof, chitosan, chitosan derivatives, polyester-polyalkylene oxide block copolymers (e.g., PLGA-PEG-PLGA, MePEG-PLGA, etc), degradable polyesters, polyanhydrides, polyorthoesters, polyphosphoesters, polyphosphazines, cyanoacrylate polymers, injectable PEG-containing formulations such as COSEAL, FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL, and other low molecular weight polymers that can be excreted.

It should be apparent to one of skill in the art that potentially any hyaluronidase inhibitor may be utilized alone, or in combination, in the practice of this embodiment as described above. Exemplary HI agents for use in combination with HA in ocular procedures include aurothiomalate; propylene glycol; dextran sulphate; fucoidan; heparin; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate, as well as analogues and derivatives of the aforementioned.

Suitable doses of these compounds may be such as to provide a steady concentration of each agent to elicit a prolonged inhibitory effect on hyaluronidase. These concentrations are approximately and may be adjusted depending on the potency of the compound and duration of effect required: aurothiomalate 10 mM, indomethacin 1 mg/ml, heparin 1 mg/ml, sulphated polysaccharides 2 mg/ml, and propylene glycol, TRITON X-100, PEG; SPAN, PLURONIC L101 and carboxymethyl cellulose all at 10 mg/ml. In order to attain this concentration a dose of approximately 10 times that required dose per ml may be needed (e.g., a total weight of 10 mg indomethacin, 20 mg sulphated polysaccharides, 100 mg propylene glycol, TRITON, PEG, SPAN, PLURONIC or carboxymethyl cellulose) in an area that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health.

6. HI-Loaded Hyaluronic Acid for Intra-Articular Injection

Osteoarthritis (OA) is a painful degenerative joint condition that affects millions of Americans. Although the exact cause of OA is unknown, possible causes include injury, age, congenital predisposition and obesity. Hyaluronic acid, which is a normal element of joint synovial fluid, lubricates the joint surface during normal activities (resting, walking) and helps prevent mechanical damage and decrease shock on the joint in high impact activities (such as running, jumping). In patients with OA, the elasticity and viscosity of the synovial fluid and the synovial hyaluronic acid concentration are reduced. It is believed that this contributes to the breakdown of the articular cartilage within the joint. Intra-articularly administered HA (typically sodium hyaluronate) penetrates the articular cartilage surface, the synovial tissue, and the capsule of the joint for a period of time after injection. By injecting hyaluronic acid into the joint (known as viscosupplementation), it is possible to partially restore the normal environment of the synovial fluid, reduce pain, and potentially prevent further damage and disability.

HA-containing materials typically are administered as an intra-articular injection (as either a single treatment or a course of repeated treatment cycles) for the treatment of painful osteoarthritis of the knee in patients who have insufficient pain relief from conservative therapies. Occasionally, other joints such as hips (injected under fluoroscopy), ankles, shoulders and elbow joints, are also injected with HA to relieve the symptoms of the disease in those particular joints. Depending upon the particular commercial product, the HA material is injected into the joint once a week for 5 to 6 consecutive weeks. When effective, patients may report that they receive symptomatic relief for a period of 6 months or more—at which time the cycle may be repeated to prolong the activity of the therapy. Despite the sustained benefit in some patients, the injected HA is rapidly cleared (removed) from the joint by the body over a period of several days. Prolonging the residence time of the HA in the joint by inhibiting its breakdown may be expected to enhance its efficacy and increase the duration of symptomatic relief.

In one aspect, the compositions of the present invention may be used for the management of osteoarthritis in animals (e.g., horses).

In the present invention, an HI is added to the intra-articular HA-containing implant or composition to decrease the rate of degradation of the HA and prolong the composition/implant's activity in vivo beyond that seen with HA alone (e.g., consistently longer than 6 months in many patients and longer than 1 year in some patients). The total dose delivered, the rate of dose release, and the duration of drug release from the matrix can be tailored to significantly prolong the activity of the HA implant as required.

Numerous commercially available HA-containing materials may be combined with an HI, including, for example, SYNVISC is an elastoviscous fluid containing hylan (a derivative of sodium hyaluronate) derived from chicken combs); ORTHOVISC, a highly purified, high molecular weight, high viscosity injectable form of HA, HYALGAN (from Fidia/Sanofi-Synthelabo); and HPS and SUPARTZ (from Seikagaku/Smith & Nephew). It should be noted that some HA products (notably HYVISC by Boehringer Ingelheim Vetmedica, St. Joseph, Mo.) are used in veterinary applications (typically in horses to treat osteoarthritis and lameness).

Representative examples of hyaluronic acid compositions used in visco-supplementation are described in U.S. Pat. Nos. 6,654,120, 6,645,945, and 6,635,287.

The HI may be combined with a polymer system to provide sustained release of the agent. Materials suitable for delivery of a HI agent in combination with HA in the management of OA include non-degradable and degradable materials; although degradable polymers are preferred. Suitable degradable materials include, but are not limited to, crosslinked materials of PEG, gelatin, collagen, GELFOAM, polysaccharides, carbohydrates, proteins (e.g., albumin, casein, whey proteins, plant proteins, fish proteins, etc.), alginates, starch, cellulose derivatives (HPC and the like), cellulose, cellulose esters, blends and copolymers thereof, chitosan, chitosan derivatives, polyester-polyalkylene oxide block copolymers (e.g., PLGA-PEG-PLGA, MePEG-PLGA, and the like), degradable polyesters, polyanhydrides, polyorthoesters, polyphosphoesters, polyphosphazines, and cyanoacrylate polymers. Particularly useful degradable polymers for use in the practice of this invention include injectable PEG-containing formulations such as COSEAL, FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL; and other low molecular weight polymers that can be excreted. Additional compositions include blends and copolymers of the agents listed above.

It should be apparent to one of skill in the art that potentially any hyaluronidase inhibitor may be utilized alone, or in combination, in the practice of this embodiment as described above. Exemplary HI agents for use in combination with HA in the management of osteoarthritis include aurothiomalate; propylene glycol; dextran sulphate; fucoidan; heparin; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate; as well as analogues and derivatives of the aforementioned. The following compositions are ideally suited for use in this indication:

Suitable doses of these compounds may be such as to provide a steady concentration of each agent to elicit a prolonged inhibitory effect on hyaluronidase. These concentrations are approximately and may be adjusted depending on the potency of the compound and duration of effect required: aurothiomalate 10 mM, indomethacin 1 mg/ml, heparin 1 mg/ml, sulphated polysaccharides 2 mg/ml, and propylene glycol, TRITON X-100, PEG SPAN, PLURONIC L101 and carboxymethyl cellulose all at 10 mg/ml. In order to attain this concentration a dose of approximately 10 times that required dose per ml may be needed (e.g., a total weight of 10 mg indomethacin, 20 mg sulphated polysaccharides, 100 mg propylene glycol, TRITON, PEG, SPAN, PLURONIC or carboxymethyl cellulose) in an area that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore if the inhibitor was released in a controlled manner from a polymeric dosage form then the applied total dose may be calculated by one skilled in the art based on inhibitor release profiles, site of application, turn over of body fluid in that area and other parameters such as age and general health.

7. HI-Loaded Hyaluronic Acid Bulking Agents for GERD

HA-based injectables are used for the management of gastroesophageal reflux disease (GERD). GERD occurs when the lower esophageal sphincter (the muscle between the stomach and the esophagus) is unable to prevent the contents of the stomach from refluxing back into the esophagus. Gastric acid and enzymes are quite corrosive to the epithelial lining of the esophagus and can cause erosions, ulceration, scarring and narrowing of the esophagus. Repetitive reflux into the esophagus can result in irreversible injury and also predisposes the patient to the development of a particular form of esophageal cancer. Injection of an HA-bulking agent into the vicinity of the lower esophageal sphincter (LES) can restore the structure of the tissue and reduce backflow into the esophagus. The HA-bulking agent is typically administered through direct injection under endoscopic vision. As occurs with virtually all HA-based procedures, the principle problem is degradation of the implant, which limits the longevity of the treatment. A repeat intervention, with either re-injection of HA (or another biomaterial such as collagen) or open surgical reinforcement of the sphincter, is required when the hyaluronic acid loses its structural integrity and can no longer maintain the LES. A representative example of a HA-based bulking agent for treatment of GERD is DEFLUX from Q-Med/Priority Healthcare. Representative examples of hyaluronic acid compositions used in GERD surgery are described in U.S. Pat. Nos. 6,736,823, 6,736,854, 6,316,011.

In the present invention, an HI is added to the HA-containing implant or composition alone, or in a sustained-release form, to decrease the rate of degradation of the hyaluronic acid and prolong the composition/implant's activity in vivo beyond that seen with HA alone (e.g., consistently longer than 6 months in >75% of patients and longer than 1 year in >35% of patients). The total dose delivered, the rate of dose release, and the duration of HI release from the matrix can be tailored to significantly prolong the activity of the HA implant as required.

The HI may be combined with a polymer system to provide sustained release of the agent. The material suitable for delivery of a HI agent in combination with HA in the management of GERD can be a non-degradable or a degradable material. Suitable degradable materials include, but are not limited to, crosslinked materials of PEG, gelatin, collagen, GELFOAM, polysaccharides, carbohydrates, proteins (e.g., albumin, casein, whey proteins, plant proteins, fish proteins etc), alginates, starch, cellulose derivatives (e.g., HPC), cellulose, cellulose esters, blends and copolymers thereof, chitosan, chitosan derivatives, polyester-polyalkylene oxide block copolymers (e.g., PLGA-PEG-PLGA, MePEG-PLGA, and the like), degradable polyesters, polyanhydrides, polyorthoesters, polyphosphoesters, polyphosphazines, and cyanoacrylate polymers. Particularly useful degradable polymers for use in the practice of this invention include injectable PEG-containing formulations such as COSEAL, FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL; and other low molecular weight polymers that can be excreted.

Suitable non-degradable materials for delivery of an HI in combination with HA for the management of GERD include crosslinked compositions that comprise PVA, PVP, polyacrylamide, methyl methacrylate (MMA) and methyl methacrylate styrene (MMA-styrene) which when mixed together form polymethyl methacrylate (PMMA) or bone cement (e.g., SIMPLEX P made by Stryker Howmedica, ZIMMER REGULAR and ZIMMER LOW VISCOSITY CEMENT made by Zimmer, PALACOS made by Smith and Nephew, CMW-1 and CMW-2 made by Wright Medical, DEPUY ENDURANCE made by DePuy), synthetic cancellous bone void fillers (e.g. CORTOSS, Orthovita, Inc.), pHEMA, poly(vinyl PEG), poly(styrene sulfonate), poly(acrylic acid), poly(methacrylic acid), as well as other polymers that are known in the literature to form hydrogels. Additional compositions include blends and copolymers of the agents listed above.

It should be apparent to one of skill in the art that potentially any hyaluronidase inhibitor may be utilized alone, or in combination, in the practice of this embodiment as described above. Exemplary HI agents for use in combination with HA in the management of GERD include aurothiomalate; propylene glycol; heparin; dextran sulphate; fucoidan; carboxymethyl cellulose; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate; as well as analogues and derivatives of the aforementioned. The following compositions are ideally suited for use in this indication:

Suitable doses of these compounds may be such as to provide a steady concentration of each agent to elicit a prolonged inhibitory effect on hyaluronidase. These concentrations are approximate and may be adjusted depending on the potency of the compound and duration of effect required: aurothiomalate 10 mM, indomethacin 1 mg/ml, heparin 1 mg/ml, sulphated polysaccharides 2 mg/ml, and propylene glycol, TRITON X-100, PEG, SPAN, PLURONIC L101 and carboxymethyl cellulose all at 10 mg/ml. In order to attain this concentration a dose of approx 10 times that required dose per ml may be needed (e.g., a total weight of 10 mg indomethacin, 20 mg of sulphated polysaccharides, 100 mg of propylene glycol, TRITON, PEG, SPAN, PLURONIC or carboxymethyl cellulose) in an area that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health

8. HI-Loaded Hyaluronic Acid Bulking Agents for Urinary Incontinence

Injectable hyaluronic acid is often used in the treatment of urinary incontinence. The embodiment described below details compositions of hyaluronidase inhibitor-loaded HA products and methods for their use in the treatment of this common medical condition.

Briefly, incontinence, or the involuntary loss of urine, is a common medical condition which affects 20% of women and 1-2% of men at some point in their lifetime. The most common form of incontinence is stress incontinence, or the inadvertent leakage of urine in response to activities that cause an increase in intra-abdominal pressure (such as sneezing, coughing, or straining). This occurs when intravesical pressure (pressure in the bladder) exceeds the pressure in the urethra, forcing urine from the bladder and into the urethra in the absence of detrusor (bladder muscle) contraction. Several conditions are thought to result in stress incontinence, including:

(1) Descent of the bladder neck and internal urethral sphincter out of the abdomen.

(2) Intrinsic urethral sphincter failure due to trauma, surgery, childbirth or malignancy.

Corrective measures are aimed principally at supporting the proximal urethral and bladder neck within the abdominal cavity by surgical or non-surgical means. A second approach involves the use of urethral bulking agents (including HA) designed to increase urethral pressure and reduce stress incontinence.

Although periurethral and transurethral HA injections have been used with success in the management of stress incontinence, the majority of cases require more than one treatment due to the limited durability of the HA implant. Utilizing a HI-loaded HA injection can sustain the activity of the implant and reduce the need for, and frequency of, subsequent periurethral and transurethral injections.

Several commercially available HA-based products are available for the management of stress incontinence. A representative example of a HA-based vesicoureteral reflux (urinary incontinence) product is DEFLUX from Q-Med/Priority Healthcare. Unfortunately, HA begins to degrade within a few weeks and degrades completely within several months. Although the percentage of patients showing improvement in their incontinence after therapy initially ranges from 58-100%, HA resorption results in the need to repeat the procedure within the above mentioned time intervals in the majority of patients. In the present invention, an HI is added to the HA-based injectable alone, or in a sustained-release form, to decrease the rate of degradation of the implant and prolong its activity in vivo beyond that seen with HA alone (i.e., consistently greater than 1 year in the majority of patients).

Representative examples of hyaluronic acid compositions used in urinary incontinence are described in U.S. Pat. Nos. 6,605,294; 6,699,471; and 6,423,332.

In the present invention, an HI is added to the HA-containing implant or composition alone, or in a sustained-release form, to decrease the rate of degradation of the hyaluronic acid and prolong the composition/implant's activity in vivo beyond that seen with HA alone (e.g., consistently longer than 6 months in >75% of patients and longer than 1 year in >35% of patients). The total dose delivered, the rate of dose release, and the duration of HI release from the matrix can be tailored to significantly prolong the activity of the HA implant as required.

The HI may be combined with a polymer system to provide sustained release of the agent. The materials suitable for delivery of an HI agent in combination with HA for the management of urinary incontinence can be a non-degradable or a degradable material. Suitable degradable materials include, but are not limited to, crosslinked materials of PEG, gelatin, collagen, GELFOAM, polysaccharides, carbohydrates, proteins (e.g., albumin, casein, whey proteins, plant proteins, fish proteins etc), alginates, starch, cellulose derivatives (HPC etc), cellulose, cellulose esters, blends and copolymers thereof, chitosan, chitosan derivatives, polyester-polyalkylene oxide block copolymers (e.g., PLGA-PEG-PLGA, MePEG-PLGA, etc), degradable polyesters, polyanhydrides, polyorthoesters, polyphosphoesters, polyphosphazines, and cyanoacrylate polymers. Particularly useful degradable polymers for use in the practice of this invention include injectable PEG-containing formulations such as COSEAL, FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL; and other low molecular weight polymers that can be excreted.

Suitable non-degradable materials for delivery of an HI in combination with HA for the management of urinary incontinence include crosslinked compositions that comprise PVA, PVP, polyacrylamide, methyl methacrylate (MMA) and methyl methacrylate styrene (MMA-styrene) which when mixed together form polymethyl methacrylate (PMMA) or bone cement (e.g., SIMPLEX P made by Stryker Howmedica, ZIMMER REGULAR and ZIMMER LOW VISCOSITY CEMENT made by Zimmer, PALACOS® made by Smith and Nephew, CMW-1 and CMW-2 made by Wright Medical, DEPUY ENDURANCE made by DePuy), synthetic cancellous bone void fillers (e.g., CORTOSS™, Orthovita), pHEMA, poly(vinyl PEG), poly(styrene sulfonate), poly(acrylic acid), poly(methacrylic acid), as well as other polymers that are known in the literature to form hydrogels. Additional compositions include blends and copolymers of the agents listed above.

a. Transurethral Technique:

Regardless of the formulation utilized, administration of an HI-loaded HA transurethral injection may proceed in the following manner. A single use, pre-loaded syringe with a fine gauge needle (23 gauge transurethral injection needle with a stabilizing cannula) containing several mls of the implant material is used. The patient is placed in the lithotomy position and 10 ml of 2% lidocaine is inserted into the urethra for anesthesia. In women, the bladder neck is visualized cystoscopically. Via the injection port of the cystoscope, the needle is inserted at the 4 o'clock position, at a sharp angle, 1-1.5 cm distal to the bladder neck, into the plane just beneath the bladder mucosa. The needle is then advanced with the cystoscope parallel to the long axis of the urethra until it lies just below the mucosa of the bladder neck. The HI-loaded HA is injected slowly into this site. The procedure is then repeated at the 8 o'clock position. Methylene blue, or other nontoxic coloring agents, can be added to the implant to assist with visualization of the injection.

b. Periurethral Injection

Periurethral injection of an HI-loaded HA injection can also be used for the treatment of incontinence. A single use, pre-loaded syringe with a fine gauge needle (periurethral injection needle) containing several mls of the implant material is used. The patient is placed in the lithotomy position, 10 ml of 2% lidocaine is inserted into the urethra for anesthesia, and the bladder neck is visualized cystoscopically (in men the urethra can also be visualized via suprapubic cystoscopic approach). The needle is inserted transvaginally or suprapubically into the area immediately adjacent and lateral to the urethra. When it reaches the appropriate position near the bladder neck (as seen cystoscopically and described above), the HI-loaded HA is injected slowly into this site. Methylene blue, or other nontoxic coloring agents, can be added to the implant to assist with visualization of the injection.

It should be apparent to one of skill in the art that potentially any hyaluronidase inhibitor may be utilized alone, or in combination, in the practice of this embodiment as described above. Exemplary HI agents for use in combination with HA in the management of urinary incontinence include aurothiomalate; propylene glycol; dextran sulphate; fucoidan; carboxymethyl cellulose; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate; as well as analogues and derivatives of the aforementioned. The following compositions are ideally suited for use in this indication:

Suitable doses of these compounds may be such as to provide a steady concentration of each agent to elicit a prolonged inhibitory effect on hyaluronidase. These concentrations are approximate and may be adjusted depending on the potency of the compound and duration of effect required: aurothiomalate 10 mM, indomethacin 1 mg/ml, heparin 1 mg/ml, sulphated polysaccharides 2 mg/ml, and propylene glycol, TRITON X-100, PEG, SPAN, PLURONIC L101 and carboxymethyl cellulose all at 10 mg/ml. In order to attain this concentration a dose of approx 10 times that required dose per ml may be needed (e.g., a total weight of 10 mg indomethacin, 20 mg of sulphated polysaccharides, 100 mg of propylene glycol, TRITON, PEG, SPAN, PLURONIC or carboxymethyl cellulose) in an area that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health.

9. HI-Loaded HA Bulking Agents for Fecal Incontinence

HA-based injectables may also be used in the local management of fecal incontinence. Fecal incontinence is a common and socially disabling condition that affects up to 11% of North American adults. Incontinence to flatus or feces can be caused by a variety of factors, but is more common in women where the anal sphincter can be damaged during child birth (especially those who have suffered a third degree vaginal tear, required forceps, had large babies, and/or experienced long labor as part of a vaginal delivery). Although the etiology of fecal incontinence is often multifactorial, causes include sphincter injury (obstetric, surgical, accidental), anorectal disease (hemorrhoids, rectal prolapse, inflammatory bowel disease, fistulas, tumors, colon resection, fecal impaction, diarrhea), congenital (spina bifida, meningocele, Hirshsprung's disease), idiopathic, or behavioral (resistance to defecation, dementia, mental retardation). Passive fecal incontinence (i.e., occurring without the patient's awareness) is primarily due to dysfunction of the internal anal sphincter, while urge fecal incontinence (the inability to voluntarily suppress defecation) is usually due to external-anal sphincter dysfunction.

Corrective measures are initially conservative or directed towards eliminating the underlying cause (if readily evident). In a significant number of patients, no defined cause can be identified and surgical repair of the internal or external anal sphincter is often attempted. Unfortunately, over 50% of these patients will not achieve a long-term successful outcome and will require another form of treatment. Those who have failed surgery, patients who do not wish to have surgery, and patients who cannot be operated on for medical reasons are all candidates for injectable sphincter augmentation. In this procedure a bulking agent, such as HA, is injected into the region around the internal or external sphincter to increase sphincter pressure and reduce fecal incontinence.

Although peri-anal-sphincter HA injections have been used with success in the management of fecal incontinence, the majority of cases require more than one treatment due to the limited durability of the HA implant. Utilizing a HI-loaded HA injection can sustain the activity of the implant and reduce the need for, and frequency of, peri-anal injections. Several commercially available HA-based products can be used in the management of fecal incontinence. A variety of HA-based bulking agents may be used in the treatment of fecal incontinence, include injectable bulking agents. A representative example of an HA-based vesicoureteral reflux product that can also be used in fecal incontinence is DEFLUX from Q-Med/Priority Healthcare, which is comprised of particles of crosslinked dextran in a solution of hyaluronic acid.

Representative examples of hyaluronic acid compositions used in fecal incontinence are described in U.S. Pat. Nos. 6,129,761 and 5,490,984.

Unfortunately, HA begins to degrade within a few weeks and degrades completely within several months. Although the percentage of patients showing improvement in their incontinence after therapy is high, HA resorption results in the need to repeat the procedure in the majority of patients. In the present invention, an HI is added to the HA-based injectable alone, or in a sustained-release form, to decrease the rate of degradation of the implant and prolong its activity in vivo beyond that seen with HA alone (i.e., consistently greater than 1 year in the majority of patients). The total dose delivered, the rate of dose release, and the duration of HI release from the matrix can be tailored to significantly prolong the activity of the HA implant as required.

The HI may be combined with a polymer system to provide sustained release of the agent. The materials suitable for delivery of an HI agent in combination with HA for the management of fecal incontinence can be a non-degradable or a degradable material. Suitable degradable materials include, but are not limited to, crosslinked materials of PEG, gelatin, collagen, GELFOAM, polysaccharides, carbohydrates, proteins (e.g., albumin, casein, whey proteins, plant proteins, fish proteins etc), alginates, starch, cellulose derivatives (HPC etc), cellulose, cellulose esters, blends and copolymers thereof, chitosan, chitosan derivatives, polyester-polyalkylene oxide block copolymers (e.g., PLGA-PEG-PLGA, MePEG-PLGA, etc), degradable polyesters, polyanhydrides, polyorthoesters, polyphosphoesters, polyphosphazines, and cyanoacrylate polymers. Particularly useful degradable polymers for use in the practice of this invention include injectable PEG-containing formulations such as COSEAL, FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL; and other low molecular weight polymers that can be excreted.

Examples of non-degradable materials for delivery of an HI in combination with HA for the management of fecal incontinence include crosslinked compositions that comprise PVA, PVP, polyacrylamide, methyl methacrylate (MMA) and methyl methacrylate styrene (MMA-styrene) which when mixed together form polymethyl methacrylate (PMMA) or bone cement (e.g., SIMPLEX P made by Stryker Howmedica, ZIMMER REGULAR and ZIMMER LOW VISCOSITY CEMENT made by Zimmer, PALACOS made by Smith and Nephew, CMW-1 and CMW-2 made by Wright Medical, DEPUY ENDURANCE made by DePuy), synthetic cancellous bone void fillers (e.g., CORTOSS, Orthovita), pHEMA, poly(vinyl PEG), poly(styrene sulfonate), poly(acrylic acid), poly(methacrylic acid), as well as other polymers that are known in the literature to form hydrogels. Additional compositions include blends and copolymers of the materials listed above.

Peri-anal-sphincter injection of HI-loaded HA is performed in the following manner. A single use, pre-loaded syringe with a fine gauge needle containing several mls of the implant material is used. Approximately 10 ml of 2% lidocaine is inserted into the perineal skin or the rectal mucosa depending upon the region of injection selected. The needle is inserted through the skin or the rectal mucosa into the submucosal plane surrounding the anal sphincter. When needle reaches the appropriate position, the HI-loaded HA is injected slowly into the site (typically, in 3 injections placed circumferentially, trans-sphincterally, entering away from the anal margin and injecting at, or just above, the dentate line) until symmetry is achieved around the anal canal. Methylene blue, or other nontoxic coloring agents, can be added to the implant to assist with visualization of the injection.

It should be apparent to one of skill in the art that potentially any hyaluronidase inhibitor may be utilized alone, or in combination, in the practice of this embodiment as described above. Exemplary HI agents for use in combination with HA in the management of fecal incontinence include aurothiomalate; carboxymethyl cellulose; dextran sulphate; fucoidan; propylene glycol; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4-methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate; as well as analogues and derivatives of the aforementioned. The following compositions are ideally suited for use in this indication:

Suitable doses of these compounds may be such as to provide a steady concentration of each agent to elicit a prolonged inhibitory effect on hyaluronidase. These concentrations are approximate and may be adjusted depending on the potency of the compound and duration of effect required: indomethacin 1 mg/ml, heparin 1 mg/ml, sulphated polysaccharides 2 mg/ml, and propylene glycol, TRITON X-100, PEG, SPAN, PLURONIC L101 and carboxymethyl cellulose all at 10 mg/ml. In order to attain this concentration a dose of approximately 10 times that required dose per ml may be needed (e.g., a total weight of 10 mg indomethacin, 20 mg sulphated polysaccharides, 100 mg propylene glycol, TRITON, PEG, SPAN, PLURONIC or carboxymethyl cellulose) in an area that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health.

In one aspect, the HI is a gold compound such as auranofin, aurothiomalate and sodium aurothiomalate, or gold sodium thiosulphate. Doses of these compounds may be such as to provide a steady concentration of each agent to elicit a prolonged inhibitory effect on hyaluronidase. The concentrations of these agents may be equal or greater than those shown in the examples, which is approximately 10 mM. In order to attain this concentration, a dose of approximately 10 times that required dose per ml may be needed (e.g., a total concentration of 10 mM aurothiomalate in an area that may be exposed to a few ml of aqueous body fluid. So, for example, if 1 ml of an HA solution was injected where the injection fluid may be exposed to perhaps 2 ml of interstitial fluid diffusing past the area then a dose of 100 mg of each of these inhibitors would be recommended to ensure attainment of a dose of 10 mg per ml for some time after. The dosing needs depend largely of the injection volume and the site of application. At sites with a higher fluid turn over, more hyaluronidase inhibitor may be given. Furthermore, if the inhibitor was released in a controlled manner from a polymeric dosage form then the applied total dose may be calculated by one skilled in the art based on inhibitor release profiles, site of application, turn over of body fluid in that area and other parameters such as age and general health.

10. HI-Loaded HA Coatings for Medical Devices

HA can be used as a coating for medical devices to enhance the biocompatibility and/or lubricity of the device surface. The HA can be coated directly onto the medical device surface or the device can be coated onto the device surface and then further modified to enhance the adhesion and/or retention of the HA on the device surface. Modifications to the HA can include crosslinking. The crosslinking can be accomplished by using a process for chemical crosslinking, ionic crosslinking, physical crosslinking or radiation-induced crosslinking. The HA coating can further comprise a hyaluronidase inhibitor such that the hyaluronidase-induced degradation of the HA coating is reduced. These HA-HI compositions can be used to coat any type medical device, including without limitation stents, catheters, electrical leads such as pacemaker leads, ocular implants, intraocular lenses, contact lenses, shunts, bypass grafts, stent-grafts, sutures, and bone fixation devices.

The HI may be combined with a polymer system to provide sustained release of the agent. The materials suitable for delivery of an HI agent in combination with HA can be a non-degradable or a degradable material. Suitable degradable materials include, but are not limited to, crosslinked materials of PEG, gelatin, collagen, GELFOAM, polysaccharides, carbohydrates, proteins (e.g., albumin, casein, whey proteins, plant proteins, fish proteins etc), alginates, starch, cellulose derivatives (HPC etc), cellulose, cellulose esters, blends and copolymers thereof, chitosan, chitosan derivatives, polyester-polyalkylene oxide block copolymers (e.g., PLGA-PEG-PLGA, MePEG-PLGA, and the like), degradable polyesters, polyanhydrides, polyorthoesters, polyphosphoesters, polyphosphazines, and cyanoacrylate polymers. Particularly useful degradable polymers for use in the practice of this invention include injectable PEG-containing formulations such as COSEAL, FOCALSEAL, SPRAYGEL, DURASEAL or a composition that includes a 4-armed thiol PEG (10K), a 4-armed NHS PEG(10K) and methylated collagen, such as described in U.S. Pat. Nos. 5,874,500; 6,051,648; 6,166,130 and 6,312,725, fibrinogen-containing formulations such as FLOSEAL or TISSEAL, REPEL or FLOWGEL; and other low molecular weight polymers that can be excreted. Suitable non-degradable materials for delivery of an HI in combination with HA include cross-linked compositions that comprise PVA, PVP, polyacrylamide, methyl methacrylate (MMA) and methyl methacrylate styrene (MMA-styrene) which when mixed together form polymethyl methacrylate (PMMA) or bone cement (e.g., SIMPLEX P made by Stryker Howmedica, ZIMMER REGULAR and ZIMMER LOW VISCOSITY CEMENT made by Zimmer, PALACOS made by Smith and Nephew, CMW-1 and CMW-2 made by Wright Medical, DEPUY ENDURANCE made by DePuy), synthetic cancellous bone void fillers (e.g., CORTOSS, Orthovita), pHEMA, poly(vinyl PEG), poly(styrene sulfonate), poly(acrylic acid), poly(methacrylic acid), as well as other polymers that are known in the literature to form hydrogels. Additional compositions include blends and copolymers of the agents listed above.

It should be apparent to one of skill in the art that potentially any hyaluronidase inhibitor may be utilized alone, or in combination, in the practice of this embodiment as described above. Exemplary HI agents for use in combination with HA in the medical device coatings include: aurothiomalate; heparin; fucoidan; dextran sulphate; propylene glycol; carboxymethylcellulose; flavonoids such as condensed tannin, tannic acid, kaempferol, quercetin, apeginin, hydrangenols from hydrangea, curcumins from the spice cumin, glychyrrhizin, isoliquiritin, glabridin, liquirtigenin, rhamnoliquirtin, neoliquirtin, licoflavonol, licoisoflavones A & B, licoisoflavone, formononetin glabrol, glabrone, glabrene, hispglabridin A, hispglabridin B, baicalein, tranilast, silybin, phloretin, taxifolin, diadzein (4′,7-dihydroxyisoflavone), tectorigenin (4′,7-dihydroxy-6-methoxyisoflavone, luteolin, xanthohumol, isoxanthohumol, genistein, naringenin, chalconaringenin, myricetin, phosphorylated hesperidin, biochanin A, morin, phloretin, silymarin, 4-phenyl-coumarin, 7-fluoro-4′-hydroxyflavone-4′-chloro-4,6-dimethoxychalcone, sodium flavonone-7-sulphate, sodium-5-hydroxyflavone-7-sulphate, 4′-chloro-4,6-dimethoxychalcone; anti-inflammatory agents such as indomethacin, aescin, traxanox, salicylates, eicosatrienoic acid, glychyrrhizin; agents that modulate allergic reactions such as disodium cromoglycate (DSCG), tranilast, liquiritigenin, isoliquiritigenin, baicalein, sodium polystyrene sulfonate (N-PSS), saccharic acid, chondroitin sulphate A-derived oligosaccharide (ChSAO), phenylbutazone, oxyphenbutazone, γ-linolenic acid, fenoprofen; phenolic compounds such as diphenylacrylic acid, diphenylpropionic acid, 3-(4-trifluoromethyl-phenyl)-3-phenylpropionic acid, 3-(4-trifluoromethyl-phenyl)-3-phenylpropionic acid, 1-(2-hydroxy-4,6-dimethoxyphenyl)-3-(4methoxyphenyl) propenone, 1-(2-hydroxy-4,6-dimethoxyphenyl)-3-(4-chlorophenyl)propenone, indole-2-carboxylic acid, norlignane, ellagitannins, and urolithin B; Vitamin C, L-ascorbic acid 6-hexadecanoate; saponins such as hederagenin; cysteamine; echinacea; rosmaric acid; guanidine hydrochloride; L-arginine; surfactants such as tetradecyl sodium sulphate, or octylphenol ethoxylate; as well as analogues and derivatives of the aforementioned.

Suitable doses of these compounds may be such as to provide a steady concentration of each agent to elicit a prolonged inhibitory effect on hyaluronidase. The concentrations of these agents may be in the micro- to millimolar range and may be adjusted depending on the potency of the compound and duration of effect required.

It should be readily evident to one of skill in the art that any of the previously described HI agents, or derivatives and analogues thereof, can be utilized to create variations of the above compositions without deviating from the spirit and scope of the invention. It should also be apparent that the HI can be utilized in a hyaluronic acid implant with or without polymer carrier and that altering the carrier does not deviate from the scope of this invention. It should also be evident that combinations of HI agents can be used to create a longer-lasting HA implant without deviating from the spirit and scope of the invention.

### EXAMPLES

### Example 1

### Inhibition of Hyaluronic Acid Degradation Using the Hyaluronic Acid Viscometry Assay

A viscometry assay (Hyaluronic Acid Visometry Assay) was used to determine the effect of various compounds on the degradation of hyaluronic acid (HA) by hyaluronidase (see, e.g., “Rheological Study on Mixtures of Different Molecular Weight Hyaluronates,” Berriaud, N., et al., Int. J. Biol. Macromol. (1994); 16 (3): p. 137-142 and “Determination of Extracellular Matrix Degradation by Free Radicals using Viscosity Measurement of Hyaluronan,” Deguine V., et al., Clinica Chimica Acta (1997); 262(1-2): p. 147-52). The time for the sample to run through the viscometer was proportional to the viscosity of HA in the sample, which, in turn, was proportional to the molecular weight of the HA. As the enzyme (hyaluronidase) breaks down HA, the molecular weight of the polymer is reduced, the viscosity of the solution drops, and the solution runs through the viscometer more rapidly. Accordingly, shorter run times indicated lower molecular weights of HA (i.e., more breakdown of the HA by hyaluronidase), whereas longer times indicated higher molecular weights of HA (i.e., less breakdown of the HA by hyaluronidase).

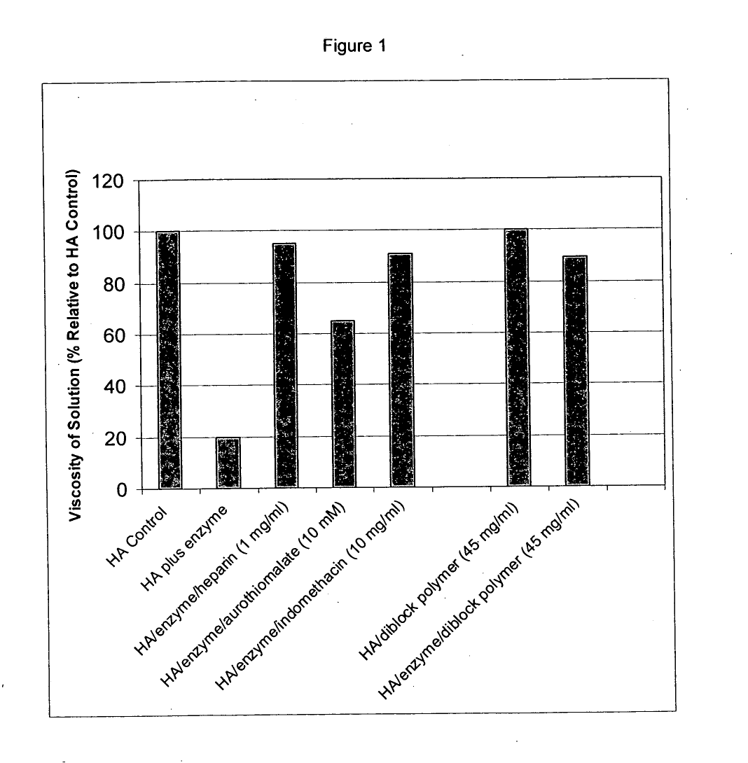

0.07% HA solutions were prepared as follows: 15 ml HA and 20 μof pH 5.5 phosphate buffer (1 M) were combined in a scintillation vial. The enzyme inhibitors (MePEG2000-PLLA (60:40) diblock copolymer, heparin, aurothiomalate, and indomethacin) were weighed into each of the vials to give the final concentrations shown in Table 1. The solutions were allowed to dissolve overnight at 37° C. The pH's were checked and adjusted to 6.0. The viscosity of the solutions was analyzed by measuring the run time through an Ubbelohde PC1 viscometer. 50 μl of hyaluronidase (4.2 mg in 620 ul of water) was added per vial (final concentration approximately 6 units/ml) and incubated overnight with shaking. A viscometer reading was recorded for each solution after overnight incubation. The results are provided in Table 1 and FIG. 1 (Note: all samples except HA control had hyaluronidase added to them). The data indicate that heparin, aurothiomalate, indomethacin and the diblock copolymer inhibit the activity of hyaluronidase, since the viscosity of the hyaluronic acid solution remains higher than that of the enzyme control.

TABLE 1

| | Viscosity of | |
|---|---|---|
| | HA solution | Time (minutes) for |
| | (% of HA | sample to run |
| Sample | Control) | through viscometer |
| HA control | 100 | 2.6 |
| HA/enzyme | 20 | 0.53 |
| HA/enzyme/heparin (1 mg/ml) | 95 | 2.46 |
| HA/enzyme/aurothiomalate (10 mM) | 65 | 1.68 |
| HA/enzyme/indomethacin (10 mg/ml) | 91 | 2.36 |
| HA/diblock polymer (45 mg/ml) | 100 | 1.93 |
| HA/enzyme/diblock polymer | 89.6 | 1.73 |
| (45 mg/ml) | | |

### Example 2

### Inhibition of Hyaluronic Acid Degradation

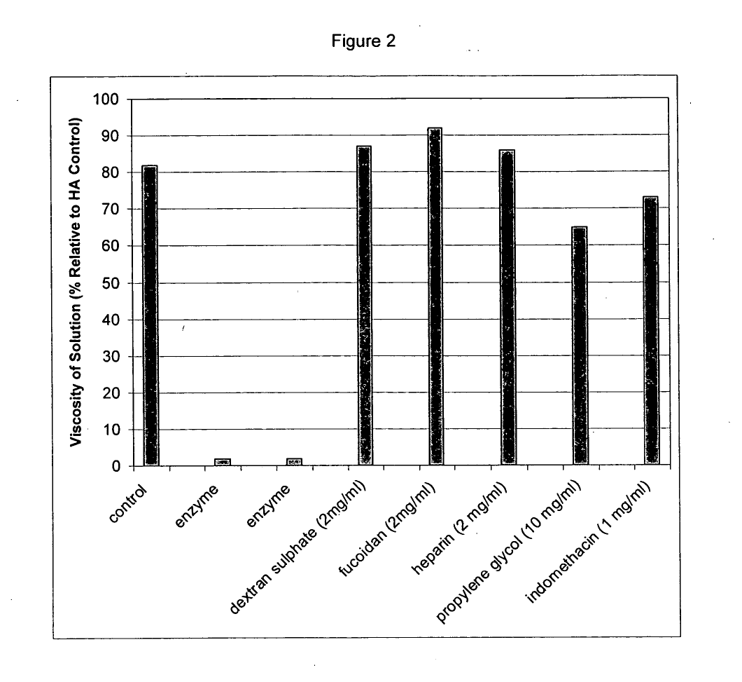

Viscometry was used to determine the effect of sulphated polysaccharides (dextran sulphate, fucoidan, and heparin), propylene glycol, and indomethacin on the degradation of HA by hyaluronidase using the procedure described in Example 1. The results are provided in Table 2 and FIG. 2. The data indicates that heparin, indomethacin, propylene glycol, dextran sulphate and fucoidan inhibit the action of hyaluronidase, since the viscosity of the hyaluronic acid solution remains higher than that of the enzyme control.

TABLE 2

| | Degradation of HA |
|---|---|
| | (% viscosity relative |
| Sample | to control t = 0) |
| HA control | 82 |
| Enzyme control | 2 |
| Enzyme control | 2 |
| Dextran sulphate (2 mg/ml) | 87 |
| Fucoidan (2 mg/ml) | 92 |
| Heparin (2 mg/ml) | 86 |
| Propylene glycol (10 mg/ml) | 65 |
| Indomethacin (1 mg/ml) | 73 |

### Example 3 INHIBITION OF HYALURONIC ACID DEGRADATION BY TRITON X-100

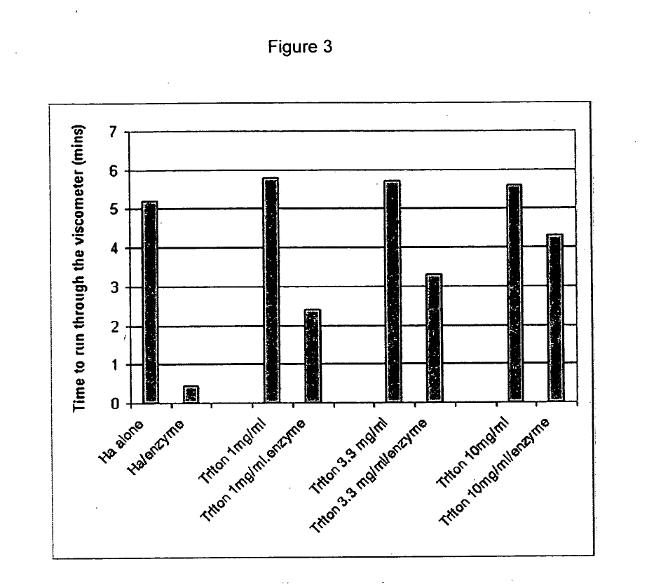

The method described in Example I was used to analyze the effect of TRITON X-100 on the inhibition of HA degradation by hyaluronidase. The results expressed in terms of time for the sample to run through the viscometer are presented in Table 3 and FIG. 3. The data indicates that TRITON X-100 inhibits the action of hyaluronidase, since the viscosity of the hyaluronic acid solution remains higher than that of the enzyme control.

TABLE 3

| | Time (minutes) for sample |
|---|---|
| Sample | to run through viscometer |
| HA control | 5.2 |
| HA/enzyme | 0.46 |
| TRITON X-100 (1 mg/ml) | 5.83 |
| TRITON X-100 (1 mg/ml)/enzyme | 2.33 |
| TRITON X-100 (3.3 mg)/ml | 5.6 |
| TRITON X-100(3.3 mg/ml)/enzyme | 3.33 |
| TRITON X-100 (10 mg/ml) | 5.46 |
| TRITON X-100 (10 mg/ml)/enzyme | 4.33 |

### Example 4

### Effect of Various Compounds on HA Degradation by Hyaluronidase

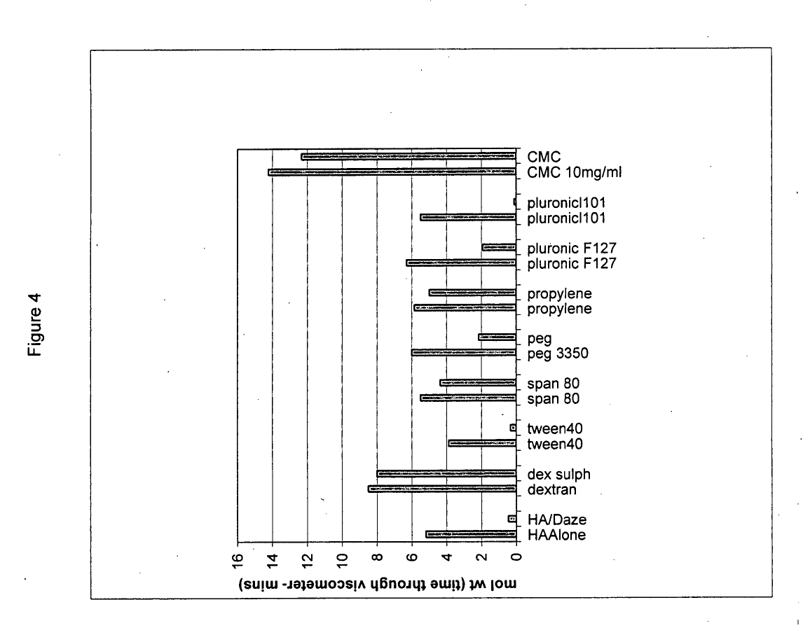

The method described in Example 1 was used to analyze the effect of dextran sulphate, TWEEN 40, SPAN 80, PEG 3350, propylene glycol, PLURONIC F127, PLURONIC L101, and carboxymethylcellulose (CMC) on the enzyme induced degradation of HA. The results are presented in Table 4 and FIG. 4. The data indicates that the agents dextran sulphate, SPAN 80, PEG, propylene glycol, PLURONIC L101 and carboxymethyl cellulose (CMC) inhibit the action of hyaluronidase, since the viscosity of the hyaluronic acid solution remains higher than that of the enzyme control. Surprisingly, the two other surfactants, TWEEN 40 and PLURONIC F 127 did not inhibit the enzyme to the same extent.

TABLE 4

| | Time (minutes) for sample to |
|---|---|
| Sample | run through viscometer |
| HA control | 5.2 |
| HA/Enzyme | 0.46 |
| Dextran sulphate (10 mg/ml) | 8.5 |
| Dextran sulphate (10 mg/ml)/enzyme | 8 |
| TWEEN 40 (10 mg/ml) | 3.88 |
| TWEEN 40 (10 mg/ml)/enzyme | 0.33 |
| SPAN 80 (10 mg/ml) | 5.5 |
| SPAN 80 (10 mg/ml)/enzyme | 4.36 |
| PEG 3350 (10 mg/ml) | 6 |
| PEG 3350 (10 mg/ml)/enzyme | 2.16 |
| Propylene glycol (10 mg/ml) | 5.85 |
| Propylene glycol (0 mg/ml)enzyme | 5 |
| PLURONIC L101 (10 mg/ml) | 6.3 |
| PLURONIC L101 (10 mg/ml)/enzyme | 1.93 |
| PLURONIC F127 (10 mg/ml) | 5.5 |
| PLURONIC F127 (10 mg/ml)/enzyme | 0.12 |
| CMC (10 mg/ml) | 14.23 |
| CMC (10 mg/ml)/enzyme | 12.33 |

### Example 5

### Dose-Response Effect of Hyaluronidase Inhibitors

Formulations of hyaluronic acid with and without various hyaluronic acid inhibitors are prepared. To test the dose-response effect of the inhibitors, hyaluronic acid formulations are made with various concentrations of inhibitor ranging in concentration, for example between 0.1 mg/mL and 20 mg/mL. The concentration of inhibitor can be adjusted depending on the potency of the inhibitor. Examples of inhibitors that may be tested are dextran sulphate, PLURONIC F127, CMC, TWEEN 40, propylene glycol, fucoidan, indomethicin, heparin and sodium aurothiomalate. Formulations are sterilized according to standard techniques. Athymic mice are obtained and prepared for subcutaneous injection of either the control hyaluronic acid formulation or inhibitor loaded formulation. The amount of formulation to be injection can be varied between 0.1 mL and 0.5 mL. The weight of the formulation is noted and the prescribed amount injected subcutaneously in a bolus to form a pouch of gel typically on the posterior back of the animal on either side of the spine. The location of the injection should be identical for each animal injected. At various periods of time, between one week and several months, animals are sacrificed, the skin at the injection site opened and the remaining formulation removed and weighed. The weight of formulation is related to dose of inhibitor, and duration of time in the animal to determine the inhibitor's effect on breakdown of hyaluronic acid formulation. The inhibitor type and dose range which lengthens the time the hyaluronic acid formulation remains intact relative to control, demonstrates an improvement in the reduction of hyaluronic acid degradation. The experiment can be varied by adding hyaluronidase to the formulation prior to injection into the animals to better control the rate of breakdown of hyaluronic acid.

### Example 6

### Preparation of Indomethacin-Loaded Microspheres by Spray Drying

3.6 grams of (poly (DL-lactide-co-glycolide) [PLGA] (85:15, Absorbable Polymers International) is dissolved in 200 ml methylene chloride. 400 mg of indomethacin is added to the polymer solution and the resulting solution is spray dried using a Buchi bench top spray drier. The spray drier parameters used are as follows: inlet temperature 50° C., outlet temperature <39° C., aspirator 100%, flow rate 700 l/hr. The collected microspheres are dried overnight under vacuum at room temperature to produce uniform, spherical particles having size ranges of less than about 10 microns (typically about 0.5 to about 2 microns).

### Example 7

### Indomethacin-Loaded Microspheres (<10 Micron) Prepared by an Oil-in Water Method

800 mg PLG (85:15, Absorbable Polymers International) is dissolved in 20 ml dichloromethane. 160 mg of indomethacin is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 mL graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

### Example 8

### Indometracin Containing Microspheres (50-100 Micron) by the Oil-in-Water Emulsion Process

Microspheres having an average size of about 50-100 microns are prepared using a 1% PVA solution and 500 rpm stirring rate using the same procedure described in Example 7.

### Example 9

### Heparin-Loaded Microspheres Prepared by a Water-in-Oil-in Water Method

Heparin (20 to 40 mg) is added in 750 uL deionized water and is vortexed for 2 minutes. 200 mg PLGA (85:15, Absorbable Polymers International) is dissolved in 7 mL methylene chloride. The aqueous drug solution is added to the methylene chloride solution and the mixture is emulsified using a POLYTRON homogenizer (speed setting 4) for 20 sec. This solution is added to 50 mL of 5% PVA solution and is homogenized with the POLYTRON homogenizer (speed setting 2) for 10 sec. The resulting double emulsion is then diluted in 100 mL of 1% PVA solution, and the system is stirred magnetically for 3 h to allow the evaporation of the methylene chloride. The microsphere solution is transferred to several disposable 50 mL graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

### Example 10

### Dextran Sulphate-Loaded Microspheres Prepared by a Water-in-Oil-in Water Method

Dextran sulphate (20 mg) is added in 750 FL deionized water and is vortexed for 2 minutes. 200 mg PDLLA (Absorbable Polymers International) is dissolved in 7 mL methylene chloride. The aqueous HI solution is added to the methylene chloride solution and the mixture is emulsified using a POLYTRON homogenizer (speed setting 4) for 20 sec. This solution is added to 50 mL of 5% PVA solution and is homogenized with the Polytron homogenizer (speed setting 2) for 10 sec. The resulting double emulsion is then diluted in 100 mL of 1% PVA solution, and the system is stirred magnetically for 3 h to allow the evaporation of the methylene chloride. The microsphere solution is transferred to several disposable 50 mL graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

### Example 11

### Fucoidan-Loaded Microspheres

200 mg PLGA (85:15, Absorbable Polymers International) is dissolved in 7 mL methylene chloride. 2 g fucoidan is placed in freezer mill tube and is cryomilled using a 6850 Freezer/Mill (AST Scientific). 40 mg of the milled fucoidan is added to the polymer solution. The solution is homogenized using a POLYTRON homogenizer (Model PT6100). 100 ml of freshly prepared 5% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 mL graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

### Example 12

### Aurothiomalate-Loaded Microspheres Prepared by a Water-in-Oil-in Water Method

Sodium aurothiomalate hydrate [Aldrich, cat: 157201] (20 to 40 mg) is added in 750 μL deionized water and is vortexed for 2 minutes. 200 mg PLGA (85:15, Absorbable Polymers International) is dissolved in 7 mL methylene chloride. The aqueous drug solution is added to the methylene chloride solution and the mixture is emulsified using a POLYTRON homogenizer (speed setting 4) for 20 sec. This solution is added to 50 mL of 5% PVA solution and is homogenized with the POLYTRON homogenizer (speed setting 2) for 10 sec. The resulting double emulsion is then diluted in 100 mL of 1% PVA solution, and the system is stirred magnetically for 3 h to allow the evaporation of the methylene chloride. The microsphere solution is transferred to several disposable 50 mL graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

### Example 13

### Preparation of Fucoidan-Loaded Microspheres by Spray Drying

3.6 grams of (poly (DL-lactide-co-glycolide) [PLGA] (85:15, Absorbable Polymers International) is dissolved in 200 ml methylene chloride. 2 g fucoidan is placed in freezer mill tube and is cryomilled using a 6850 Freezer/Mill (AST Scientific). 400 mg of the milled fucoidan is added to the polymer solution. The solution is homogenized using a POLYTRON homogenizer (Model PT6100). The resulting solution is spray dried using a Buchi bench top spray drier with the polymer solution being stirred to ensure the fucoidan did not settle out. The spray drier parameters used are as follows: Inlet temperature 50° C., outlet temperature <39° C., aspirator 100%, flow rate 700 l/hr. The collected microspheres are dried overnight under vacuum at room temperature to produce uniform, spherical particles having size ranges of less than about 10 microns (typically about 0.5 to about 2 microns).

### Example 14

### HI-Loaded SYNVISC

20 mg of the HI loaded microspheres, as prepared in Examples 6-13, are weighed into separate end-capped 3 mL syringe. The plunger is placed into the syringe and the syringe is inverted. The end-cap is removed and the plunger is pushed to the 0.1 ml mark. The end-cap is added. A 2 mL syringe containing SYNVISC is connected to the syringe containing the microspheres using a dual syringe connector. The SYNVISC is then transferred from one syringe to the next and back again at least 20 times. Once the SYNVISC/microsphere mixture is in the original syringe, the syringe is disconnected from the dual syringe connector and the formulation is ready for use.

### Example 15

### Method for Preventing Scarring Following Vertebral Disc Surgery or Laminectomy with HI-Loaded Hyaluronic Acid Orthopedic Implants

A hyaluronic acid implant that includes a hyaluronidase inhibitor is used in disc or laminectomy surgery to prevent scarring around, and compression of, the spinal nerve root in order to reduce pain and other neurological symptoms following surgery. In this indication, a hyaluronic acid containing composition containing a hyaluronidase inhibitor is injected into the tissue around a spinal nerve root as part of a surgical procedure designed to decompress an entrapped spinal nerve.

The hyaluronic acid-hyaluronidase inhibitor material is prepared as follows:

1. A 2.25 ml glass syringe containing 2 ml of hyaluronic acid (e.g., SYNVISC); HYLAN G-F 20 (Genzyme Biosurgery, Ridgefield, N.J.)—it should be noted that other sources of HA such as RESTYLANE, HYLAFORM, PERLANE, SEPRAFILM, SEPRACOAT, INTERGEL, and LUBRICOAT can also be utilized) is prepared to contain a hyaluronidase inhibitor as follows:
a. Utilizing aurothiomalate as the hyaluronidase inhibitor, the agent is incorporated into a sustained release delivery system (such as the polymeric microspheres described in Example 7) such that one achieves a concentration of 5 mg/ml of aurothiomalate in the hyaluronic acid (i.e. a total of 10 mg of aurothiomalate contained in microspheres are incorporated into 2 ml of SYNVISC). It should be noted that a range of about 0.2 mg to about 100 mg of aurothiomalate would be of clinical benefit, but about 10 mg is the preferred dosage.
b. Utilizing indomethacin as the hyaluronidase inhibitor, the agent is incorporated into a sustained release delivery system (such as the polymeric microspheres described in Examples 5, 6 and 7) such that one achieves a concentration of 1 mg/ml of indomethacin in the hyaluronic acid (i.e., a total of 2 mg of indomethacin contained in microspheres are incorporated into 2 ml of SYNVISC). It should be noted that a range of about 0.2 mg to about 20 mg of indomethacin would be of clinical benefit, but about 2 mg is the preferred dosage.
c. Utilizing propylene glycol as the hyaluronidase inhibitor, the agent is incorporated into a sustained release delivery system (such as the polymeric microspheres described in Examples 6 to 13) such that one achieves a concentration of 10 mg/ml of propylene glycol in the hyaluronic acid (i.e., a total of 20 mg of propylene glycol contained in microspheres are incorporated into 2 ml of SYNVISC). It should be noted that a range of about 0.5 mg to about 200 mg of propylene glycol would be of clinical benefit, but about 20 mg is the preferred dosage.
d. Utilizing dextran sulphate as the hyaluronidase inhibitor, the agent is incorporated into a sustained release delivery system (such as the polymeric microspheres described in Example 10) such that one achieves a concentration of 10 mg/ml of dextran sulphate in the hyaluronic acid (i.e. a total of 20 mg of dextran sulphate contained in microspheres are incorporated into 2 ml of SYNVISC). It should be noted that a range of about 0.5 mg to about 200 mg of dextran sulphate would be of clinical benefit, but about 20 mg is the preferred dosage.
e. Utilizing fucoidan as the hyaluronidase inhibitor, the agent is incorporated into a sustained release delivery system (such as the polymeric microspheres described in Example 11 and 13) such that one achieves a concentration of 5 mg/ml of fucoidan in the hyaluronic acid (i.e., a total of 10 mg of fucoidan contained in microspheres are incorporated into 2 ml of SYNVISC). It should be noted that a range of about 0.2 mg to about 100 mg of fuicoidan would be of clinical benefit, but about 10 mg is the preferred dosage.
f. Utilizing heparin as the hyaluronidase inhibitor, the agent is incorporated into a sustained release delivery system (such as the polymeric microspheres described in Example 9) such that one achieves a concentration of 1 mg/ml of heparin in the hyaluronic acid (i.e., a total of 2 mg of heparin contained in microspheres are incorporated into 2 ml of SYNVISC). It should be noted that a range of about 0.2 mg to about 100 mg of heparin would be of clinical benefit, but about 2 mg is the preferred dosage.

2. The SYNVISC/microsphere hyaluronidase inhibitor material is sterilized and administered to the patient in the manner described below. Strict aseptic administration technique must be followed during the entire surgical procedure.

Open surgery to relieve pressure on a spinal nerve typically involves resection of a ruptured lumbar disc (and portions of the bone surrounding a spinal nerve root—known as laminectomy). The patient is placed in a modified kneeling position under general anesthesia. An incision is made in the posterior midline and the tissue is dissected away to expose the appropriate interspace; the ligamentum flavum is dissected and in some cases portions of the bony lamina are removed to allow adequate visualization. The nerve root is carefully retracted away to expose the herniated fragment and the defect in the annulus. Typically, the cavity of the disc is entered from the tear in the annulus and the loose fragments of the nucleus pulposus are removed with pituitary forceps. Any additional fragments of disc sequestered inside or outside of the disc space are also carefully removed and the disc space is forcefully irrigated to remove to remove any residual fragments. If tears are present in the dura, the dura is closed with sutures that are often augmented with fibrin glue. The tissue is then closed with absorbable sutures.

As an alternative to open surgery, microlumbar disc excision (microdiscectomy) can be performed as an outpatient procedure and has largely replaced laminectomy as the intervention of choice for herniated discs. A one inch incision is made from the spinous process above the disc affected to the spinous process below. Using an operating microscope, the tissue is dissected down to the ligamentum flavum and bone is removed from the lamina until the nerve root can be clearly identified. The nerve root is carefully retracted and the tears in the annulus are visualized under magnification. Microdisc forceps are used to remove disc fragments through the annular tear and any sequestered disc fragments are also removed. As with laminectomy, the disc space is irrigated to remove any disc fragments, any dural tears are repaired and the tissue is closed with absorbable sutures. It should be noted that anterior (abdominal) approaches can also be used for both open and endoscopic lumbar disc excision. Cervical and thoracic disc excisions are similar to lumbar procedures and can also be performed from a posterior approach (with laminectomy) or as an anterior discectomy with fusion.

Unfortunately, regardless of the surgical procedure performed, in a significant number of patients, post-surgical scarring in the tissues surrounding the nerve root exerts pressure on the nerve, causes irritation, and leads to a recurrence of pain and other neurological symptoms. To reduce the incidence of this complication, the area surrounding the nerve is infiltrated with the HA/microspheric hyaluronidase inhibitor implant (described above) during open or microdiscectomy. The HA—hyaluronidase inhibitor (HI) implant prevents adjacent tissues from coming into contact with the nerve and scar tissue from forming on, and ultimately constricting around, the spinal nerve. The HA-HI implant can reduce the incidence of spinal surgery failure, prevent the recurrence of pain and neurological symptoms, and reduce the need to perform repeat surgical interventions to remove scar tissue.

### Example 16

### Method for Inhibiting Surgical Adhesions with HI-Loaded Hyaluronic Acid SURGICAL ADHESION BARRIER

A HI-containing hyaluronic acid formulation is delivered to a surface of a target tissue or organ; typically during an abdominal or gynecological surgical procedure to prevent the formation of an adhesion. The HI is added to a hyaluronic acid-containing surgical adhesion barrier (e.g., film, gel, or spray) in a sustained-release form, to decrease the rate of degradation of the HA and prolong the composition/implant's activity in vivo beyond that seen with HA alone.

Adhesions can arise as part of any surgical procedure, but are recognized to be a leading cause of bowel obstruction following abdominal surgery and a leading cause of pain and infertility following gynecological surgery. Although virtually and organ can be the site of an adhesion, the female reproductive tract (particularly the fallopian tubes) and the bowel (small and large intestine) are particularly prone to adhesion formation.

For adhesion prevention in endoscopic and open surgical procedures, a hyaluronic acid—hyaluronidase inhibitor adhesion barrier material is prepared the following way:

1. For endoscopic procedures, 2 ml of fluid hyaluronic acid (SEPRAGEL; chemically modified sodium hyaluronate/carboxymethylcellulose absorbable adhesion barrier from Genzyme Biosurgery (Ridgefield, N.J.)—it should be noted that other sources of HA such as RESTYLANE, HYLAFORM, PERLANE, SEPRACOAT, INTERGEL, and LUBRICOAT can also be utilized) is prepared to contain a hyaluronidase inhibitor as follows:
a. Utilizing aurothiomalate as the hyaluronidase inhibitor, the agent is incorporated into a sustained release delivery system (such as the polymeric microspheres described in Example 12) such that one achieves a concentration of 5 mg/ml of aurothiomalate in the hyaluronic acid (i.e., a total of 10 mg of aurothiomalate contained in microspheres are incorporated into 2 ml of SEPRAGEL). It should be noted that a range of about 0.2 mg to about 100 mg of aurothiomalate would be of clinical benefit, but about 10 mg is the preferred dosage.
b. Utilizing indomethacin as the hyaluronidase inhibitor, the agent is incorporated into a sustained release delivery system (such as the polymeric microspheres described in Examples 6, 7 and 8) such that one achieves a concentration of 1 mg/ml of indomethacin in the hyaluronic acid (i.e. a total of 2 mg of indomethacin contained in microspheres are incorporated into 2 ml of SEPRAGEL). It should be noted that a range of about 0.2 mg to about 20 mg of indomethacin would be of clinical benefit, but about 2 mg is the preferred dosage.
c. Utilizing propylene glycol as the hyaluronidase inhibitor, the agent is incorporated into a sustained release delivery system (such as the polymeric microspheres described in Examples 6 to 13) such that one achieves a concentration of 10 mg/ml of propylene glycol in the hyaluronic acid (i.e., a total of 20 mg of propylene glycol contained in microspheres are incorporated into 2 ml of SEPRAGEL). It should be noted that a range of about 0.5 mg to about 200 mg of propylene glycol would be of clinical benefit, but about 20 mg is the preferred dosage.
d. Utilizing dextran sulphate as the hyaluronidase inhibitor, the agent is incorporated into a sustained release delivery system (such as the polymeric microspheres described in Example 5) such that one achieves a concentration of about 10 mg/ml of dextran sulphate in the hyaluronic acid (i.e. a total of 20 mg of dextran sulphate contained in microspheres are incorporated into 2 ml of SEPRAGEL). It should be noted that a range of about 0.5 mg to about 200 mg of dextran sulphate would be of clinical benefit, but about 20 mg is the preferred dosage.
e. Utilizing fucoidan as the hyaluronidase inhibitor, the agent is incorporated into a sustained release delivery system (such as the polymeric microspheres described in Examples 6 and 8) such that one achieves a concentration of about 5 mg/ml of fucoidan in the hyaluronic acid (i.e. a total of 10 mg of fucoidan contained in microspheres are incorporated into 2 ml of SEPRAGEL). It should be noted that a range of about 0.2 mg to about 100 mg of fucoidan would be of clinical benefit, but about 10 mg is the preferred dosage.
f. Utilizing heparin as the hyaluronidase inhibitor, the agent is incorporated into a sustained release delivery system (such as the polymeric microspheres described in Example 4) such that one achieves a concentration of about 1 mg/ml of heparin in the hyaluronic acid (i.e. a total of about 2 mg of heparin contained in microspheres are incorporated into 2 ml of SEPRAGEL). It should be noted that a range of about 0.2 mg to about 100 mg of heparin would be of clinical benefit, but about 2 mg is the preferred dosage.

2. For open surgical procedures, a HA film containing a hyaluronidase inhibitor can be used and may be prepared the following way: a 3″×5″ or 5″×6″ hyaluronic acid film (SEPRAFILM; chemically modified sodium hyaluronate/carboxymethylcellulose absorbable adhesion barrier from Genzyme Biosurgery, Ridgefield, N.J.—it should be noted that other sources of HA films such as INTERCEED can also be utilized) is prepared to contain a hyaluronidase inhibitor as follows:
a. Utilizing aurothiomalate as the hyaluronidase inhibitor, the agent is incorporated into a sustained release delivery system (such as the polymeric microspheres described in Example 12) such that one achieves a concentration of 0.5 mg of aurothiomalate per square inch of hyaluronic acid film (i.e., a total of 7.5 mg of aurothiomalate contained in microspheres is incorporated into a 3″×5″ sheet of SEPRAFILM or a total of about 15 mg of aurothiomalate contained in microspheres is incorporated into a 5″×6″ sheet of SEPRAFILM). It should be noted that a range of about 0.01 mg to about 5 mg of aurothiomalate per square inch of hyaluronic acid film would be of clinical benefit, but about 0.5 mg/sq. in is the preferred dosage.
b. Utilizing indomethacin as the hyaluronidase inhibitor, the agent is incorporated into a sustained release delivery system (such as the polymeric microspheres described in Examples 5, 6 and 7) such that one achieves a concentration of 1 mg of indomethacin per square inch of hyaluronic acid film (i.e., a total of 15 mg of indomethacin contained in microspheres is incorporated into a 3″×5″ sheet of SEPRAFILM or a total of about 30 mg of indomethacin contained in microspheres is incorporated into a 5″×6″ sheet of SEPRAFILM). It should be noted that a range of about 0.01 mg to about 5 mg of indomethacin per square inch of hyaluronic acid film would be of clinical benefit, but about 1 mg/sq. in is the preferred dosage.
c. Utilizing propylene glycol as the hyaluronidase inhibitor, the agent is incorporated into a sustained release delivery system (such as the polymeric microspheres described in Examples 6 to 13) such that one achieves a concentration of 1 mg of propylene glycol per square inch of hyaluronic acid film (i.e., a total of 15 mg of propylene glycol contained in microspheres is incorporated into a 3″×5″ sheet of SEPRAFILM or a total of about 30 mg of propylene glycol contained in microspheres is incorporated into a 5″×6″ sheet of SEPRAFILM). It should be noted that a range of about 0.01 mg to about 20 mg of propylene glycol per square inch of hyaluronic acid film would be of clinical benefit, but about 1 mg/sq. in is the preferred dosage.
d. Utilizing dextran sulphate as the hyaluronidase inhibitor, the agent is incorporated into a sustained release delivery system (such as the polymeric microspheres described in Example 10) such that one achieves a concentration of 1 mg of dextran sulphate per square inch of hyaluronic acid film (i.e., a total of 15 mg of dextran sulphate contained in microspheres is incorporated into a 3″×5″ sheet of SEPRAFILM or a total of 30 mg of dextran sulphate contained in microspheres is incorporated into a 5″×6″ sheet of SEPRAFILM). It should be noted that a range of about 0.01 mg to about 20 mg of dextran sulphate per square inch of hyaluronic acid film would be of clinical benefit, but about 1 mg/sq. in is the preferred dosage.
e. Utilizing fucoidan as the hyaluronidase inhibitor, the agent is incorporated into a sustained release delivery system (such as the polymeric microspheres described in Examples 11 and 13) such that one achieves a concentration of 0.5 mg of fucoidan per square inch of hyaluronic acid film (i.e., a total of 7.5 mg of fucoidan contained in microspheres is incorporated into a 3″×5″ sheet of SEPRAFILM or a total of about 15 mg of fucoidan contained in microspheres is incorporated into a 5″×6″ sheet of SEPRAFILM). It should be noted that a range of about 0.005 mg to about 10 mg of fucoidan per square inch of hyaluronic acid film would be of clinical benefit, but about 0.5 mg/sq. in is the preferred dosage.
f. Utilizing heparin as the hyaluronidase inhibitor, the agent is incorporated into a sustained release delivery system (such as the polymeric microspheres described in Example 9) such that one achieves a concentration of 0.1 mg of heparin per square inch of hyaluronic acid film (i.e., a total of 1.5 mg of heparin contained in microspheres is incorporated into a 3″×5″ sheet of SEPRAFILM or a total of 3.0 mg of heparin contained in microspheres is incorporated into a 5″×6″ sheet of SEPRAFILM). It should be noted that a range of about 0.001 mg to about 5 mg of heparin per square inch of hyaluronic acid film would be of clinical benefit, but about 0.1 mg/sq. in is the preferred dosage.

3. The HA/microsphere hyaluronidase inhibitor material is sterilized and administered to the patient in the manner described below. Strict aseptic administration technique must be followed during the entire surgical procedure.

As the number of potential applicable suitable surgical procedures is vast, a generic laparoscopy and laparotomy procedure will be described. The HI-loaded adhesion barrier (gel or film as described above) is applied to the mesentery of the abdominal and pelvic organs incised, abraided or manipulated during the operation. For endoscopic procedures a sprayable formulation (such as a liquid or gel) delivered through the sideport of an endoscope is preferred. For open surgical procedures, the HI-HA sheets are applied over the disrupted intraperitoneal tissues. Regardless, the surgical field should be as dry as possible and excess fluid should be thoroughly aspirated.

For HI-HA films, the membrane is cut to the desired size and shape while handling gently with dry instruments and/or gloves. Expose 1-2 cm of the membrane through the open end of the holder included with the product. When necessary, facilitate entry into the abdominopelvic cavity by slightly curving or arching the membrane/holder. When applying, avoid contact with tissue surfaces until directly at site of application. If contact occurs, moderate application of standard irrigation solution may be used to gently dislodge membrane from unintended tissue surfaces. Allow exposed barrier to first adhere to desired position on the tissue or organ by gently pressing the membrane down with a dry glove or instrument and then withdraw the holder. Extend the barrier sufficiently beyond the margins of incision and associated surgical trauma to achieve adequate coverage. When necessary, lightly moisten the barrier with standard irrigation solution to facilitate its coverage around the contours of tissue or organs. Allow sufficient overlap of individual barrier to ensure complete, continuous coverage of traumatized tissue surface. Abdominopelvic cavity should be closed according to the standard technique of the surgeon.

The addition of the hyaluronidase inhibitor to the hyaluronic acid adhesion barrier allows the barrier to function longer in vivo and reduce the likelihood of scar tissue from forming between adjacent organs or tissues. An HI-HA implant can reduce the incidence of and/or the severity of adhesions that may form following abdominal and gynecological surgery. Adhesion reduction may prevent the occurrence of pain, bowel obstruction, and infertility, and reduce the need to perform repeat surgical interventions to remove scar tissue. It should be noted that HI-HA containing surgical adhesion barriers may be used in a variety of surgical procedures including abdominal surgery, gynecologic and pelvic surgery, spinal surgery, cardiac surgery, tendon and peripheral nerve surgery, and sinus surgery.

### Example 17

### Method for Augmenting Soft Tissue Defects with HI-Loaded Hyaluronic Acid Implants

A HA-HI implant is used for mid-to-deep dermal implantation for the correction of moderate to severe facial wrinkles and folds. An injectable hyaluronic acid composition containing a hyaluronidase inhibitor (HI) can result in increased durability (i.e., decrease the rate of degradation of the HA) and prolong the composition's activity in vivo beyond that seen with HA alone, reducing reduce the number of subsequent repeat injections.

The hyaluronic acid-hyaluronidase inhibitor material for dermal injection is prepared the following way:

1. A pre-loaded disposable glass syringe containing 0.5 ml or 1.0 ml of implant material (RESTYLANE hyaluronic acid gel material, stabilized and suspended in physiologic buffer at pH=7 and at a concentration of 20 mg/ml available from Q-Med AB, Sweden), is prepared to contain a hyaluronidase inhibitor and fitted with a sterilized fine gauge needle (30 G×½″). It should be noted that other sources of HA such as HYLAFORM (Genzyme Corporation), PERLANE, SEPRAGEL and INTERGEL can also be utilized. The hyaluronidase is incorporated into the HA injectable as follows:
a. Utilizing aurothiomalate as the hyaluronidase inhibitor, the agent is incorporated into a sustained release delivery system (such as the polymeric microspheres described in Example 12) such that one achieves a concentration of 5 mg/ml of aurothiomalate in the hyaluronic acid (i.e., a total of 5 mg of aurothiomalate contained in microspheres are incorporated into 1 ml of RESTYLANE). It should be noted that a range of about 0.2 mg to about 100 mg of aurothiomalate would be of clinical benefit, but about 5 mg is the preferred dosage.
b. Utilizing indomethacin as the hyaluronidase inhibitor, the agent is incorporated into a sustained release delivery system (such as the polymeric microspheres described in Examples 6, 7 and 8) such that one achieves a concentration of 1 mg/ml of indomethacin in the hyaluronic acid (i.e., a total of 1 mg of indomethacin contained in microspheres are incorporated into 1 ml of RESTYLANE). It should be noted that a range of about 0.2 mg to about 20 mg of indomethacin would be of clinical benefit, but about 1 mg is the preferred dosage.
c. Utilizing propylene glycol as the hyaluronidase inhibitor, the agent is incorporated into a sustained release delivery system (such as the polymeric microspheres described in Examples 6 to 13) such that one achieves a concentration of 10 mg/ml of propylene glycol in the hyaluronic acid (i.e., a total of 10 mg of propylene glycol contained in microspheres are incorporated into 1 ml of RESTYLANE). It should be noted that a range of about 0.5 mg to about 200 mg of propylene glycol would be of clinical benefit, but about 10 mg is the preferred dosage.
d. Utilizing dextran sulphate as the hyaluronidase inhibitor, the agent is incorporated into a sustained release delivery system (such as the polymeric microspheres described in Example 10) such that one achieves a concentration of 10 mg/ml of dextran sulphate in the hyaluronic acid (i.e., a total of 10 mg of dextran sulphate contained in microspheres are incorporated into 1 ml of RESTYLANE). It should be noted that a range of about 0.5 mg to about 200 mg of dextran sulphate would be of clinical benefit, but about 10 mg is the preferred dosage.
e. Utilizing fucoidan as the hyaluronidase inhibitor, the agent is incorporated into a sustained release delivery system (such as the polymeric microspheres described in Examples 11 and 13) such that one achieves a concentration of 5 mg/ml of fucoidan in the hyaluronic acid (i.e., a total of 5 mg of fucoidan contained in microspheres are incorporated into 1 ml of RESTYLANE). It should be noted that a range of about 0.2 mg to about 100 mg of fucoidan would be of clinical benefit, but about 5 mg is the preferred dosage.
f. Utilizing heparin as the hyaluronidase inhibitor, the agent is incorporated into a sustained release delivery system (such as the polymeric microspheres described in Example 9) such that one achieves a concentration of 1 mg/ml of heparin in the hyaluronic acid (i.e., a total of 1 mg of heparin contained in microspheres are incorporated into 1 ml of RESTYLANE). It should be noted that a range of about 0.2 mg to about 100 mg of heparin would be of clinical benefit, but about 1 mg is the preferred dosage.

3. The RESTYLANE/microsphere hyaluronidase inhibitor material is sterilized and administered to the patient in the manner described below. Strict aseptic administration technique must be followed during the entire surgical procedure.

The patient is placed in a sitting position with the table back slightly reclined. The patient's need for pain management is assessed. Topical lidocaine and/or prilocalne can be used for anesthesia, if necessary. The area to be treated is cleaned with alcohol or another suitable antiseptic solution.

The RESTYLANE-HI implant is administered through the thin gauge needle (30 g or 32 g). Typical usage for each treatment session is less than 2 mL per treatment site. Before injecting, press the plunger rod of the syringe carefully until a small droplet is visible at the tip of the needle. The needle is inserted at an approximate angle of 300 parallel to the length of the wrinkle or fold. The bevel of the needle should face upwards and the substance should be injected into the middle of the dermis. For mid-dermis placement, the contour of the needle should be visible but not the color of it. If RESTYLANE-HI is injected too deep or intramuscularly, the duration of the effect will be shorter. If RESTYLANE-HI is injected too superficially this may result in visible lumps and/or grayish discoloration.

The RESTYLANE-HI implant is applied with even pressure on the plunger rod, while slowly pulling the needle backwards. The wrinkle should be lifted and eliminated by the end of the injection. It is important that the injection is stopped just before the needle is pulled out of the skin to prevent material from leaking out or ending up too superficially in the skin.

It is important to only correct to 100% of the desired volume effect and not to overcorrect. With cutaneous contour deformities the best results are obtained if the defect can be manually stretched to the point where it is eliminated. The degree and duration of the correction depend on the character of the defect treated, the tissue stress at the implant site, the depth of the implant in the tissue and the injection technique. Markedly indurated defects may be difficult to correct.

The injection technique with regard to the depth of injection and the administered quantity may vary. The linear threading technique, serial puncture injections or a combination of the two have been used with success.

When the injection is completed, the treated site should be gently massaged so that it conforms to the contour of the surrounding tissues. If an overcorrection has occurred, massage the area firmly between your fingers or against an underlying superficial bone to obtain optimal results.

If so called “blanching” is observed, i.e. the overlying skin turns a whitish color, the injection should be stopped immediately and the area massaged until it returns to a normal color.

If the wrinkle needs further treatment, the same procedure should be repeated with several punctures of the skin until a satisfactory result is obtained. Additional treatment with the RESTYLANE-HI implant may be necessary to achieve the desired correction. With patients who have localized swelling the degree of correction is sometimes difficult to judge at the time of treatment. In these cases, it is better to invite the patient to a touch-up session after 1-2 weeks.

If the treated area is swollen directly after the injection, an ice pack can be applied on the site for a short period. Patients may have mild to moderate injection site reactions, which typically resolve in few days.

Examples of other suitable commercial HA products that may be combined with an HI for use in cosmetic injections include: ACHYAL from Meiji Seika Kaisha, Ltd. (Japan), JUVEDERM from L.E.A. Derm (France), MACDERMOL from Laboratoires O.R. GE V. MacDermol (France), and ROFILAN Hylan Gel from Rofil Medical International (Holland). The HA-HI composition may further comprise an anesthetic such as lidocaine, benzocaine or prilocalne and/or a neurotoxin such as a botulinum toxin.

### Example 18

### Method for Using HI-Loaded Hyaluronic Acid in Ophthalmic Surgery

A hyaluronic acid solution containing a hyaluronidase inhibitor is used in conjunction with insertion of an intraocular lens in ocular surgery.

An HI-loaded viscoelastic substance is prepared by combining a hyaluronidase inhibitor with hyaluronic acid. A variety of HA ocular products can be combined with a hyaluronidase inhibitor. For example, AMVISC, AMVISC PLUS and OCUCOAT (Bausch & Lomb) are high molecular weight, viscoelastic and injectable HA solutions used to maintain eye shape and protect delicate tissues during cataract removal, corneal transplant and glaucoma surgery. Other HA-based ophthalmic viscoelastic products include PROVIS, VISCOAT, DUOVISC, and CELLUGEL from Alcon Laboratories; HEALON, HEALON G, and HEALON 5 from Pharmacia & Upjohn, VITRAX from Allergan; BIOLON from Bio-Technology General; STAARVISC from Anika Therapeutics/Staar Surgical; SHELLGEL from Anika Therapeutics/Cytosol Opthalmics; and UNIVISC from Novartis.

Although any of the above HA products could be potentially used, in the following example HEALON GV (from Advanced Medical Optics) is combined with a hyaluronidase inhibitor in the following manner:

1. A disposable 0.85 ml and 0.55 ml glass syringe of HEALON GV (from Advanced Medical Optics; each ml of HEALON GV contains 14 mg sodium hyaluronate 7000) is prepared to contain a hyaluronidase inhibitor as follows:
a. Utilizing aurothiomalate as the hyaluronidase inhibitor, the agent is incorporated into a sustained release delivery system (such as the polymeric microspheres described in Example 12) such that one achieves a concentration of 5 mg/ml of aurothiomalate in the hyaluronic acid (i.e., a total of 4.25 mg of aurothiomalate contained in microspheres are incorporated into 0.85 ml of HEALON GV). It should be noted that a range of about 0.2 mg to about 100 mg of aurothiomalate would be of clinical benefit, but about 4.25 mg is the preferred dosage.
b. Utilizing indomethacin as the hyaluronidase inhibitor, the agent is incorporated into a sustained release delivery system (such as the polymeric microspheres described in Examples 6, 7 and 8) such that one achieves a concentration of 1 mg/ml of indomethacin in the hyaluronic acid (i.e., a total of 0.85 mg of indomethacin contained in microspheres are incorporated into 0.85 ml of HEALON GV). It should be noted that a range of about 0.05 mg to about 20 mg of indomethacin would be of clinical benefit, but about 0.85 mg is the preferred dosage.
c. Utilizing propylene glycol as the hyaluronidase inhibitor, the agent is incorporated into a sustained release delivery system (such as the polymeric microspheres described in Examples 6 to 13) such that one achieves a concentration of 10 mg/ml of propylene glycol in the hyaluronic acid (i.e., a total of 8.5 mg of propylene glycol contained in microspheres are incorporated into 0.85 ml of HEALON GV). It should be noted that a range of about 0.5 mg to about 200 mg of propylene glycol would be of clinical benefit, but about 8.5 mg is the preferred dosage.
d. Utilizing dextran sulphate as the hyaluronidase inhibitor, the agent is incorporated into a sustained release delivery system (such as the polymeric microspheres described in Example 10) such that one achieves a concentration of 10 mg/ml of dextran sulphate in the hyaluronic acid (i.e., a total of 8.5 mg of dextran sulphate contained in microspheres are incorporated into 0.85 ml of HEALON GV). It should be noted that a range of about 0.5 mg to about 200 mg of dextran sulphate would be of clinical benefit, but about 8.5 mg is the preferred dosage.
e. Utilizing fuicoidan as the hyaluronidase inhibitor, the agent is incorporated into a sustained release delivery system (such as the polymeric microspheres described in Examples 11 and 13) such that one achieves a concentration of 5 mg/ml of fucoidan in the hyaluronic acid (i.e., a total of 4.25 mg of fucoidan contained in microspheres are incorporated into 0.85 ml of HEALON GV). It should be noted that a range of about 0.2 mg to about 100 mg of fucoidan would be of clinical benefit, but about 4.25 mg is the preferred dosage.
f. Utilizing heparin as the hyaluronidase inhibitor, the agent is incorporated into a sustained release delivery system (such as the polymeric microspheres described in Example 9) such that one achieves a concentration of 1 mg/ml of heparin in the hyaluronic acid (i.e., a total of 0.85 mg of heparin contained in microspheres are incorporated into 0.85 ml of HEALON GV). It should be noted that a range of about 0.05 mg to about 100 mg of heparin would be of clinical benefit, but about 0.85 mg is the preferred dosage.

2. The HEALON GV/microsphere hyaluronidase inhibitor material is sterilized and administered to the patient in the manner described below. Strict aseptic administration technique must be followed during the entire surgical procedure.

Viscoelastic solutions of HA have been used to act as a tissue lubricant and also to maintain the volume of the eye fluid during surgery on the inside of the eye (e.g., as a vitreous substitute during cataract extraction surgery, intraocular lens implantation, retinal reattachment, phacoemulsification surgery, corneal transplantation, and glaucoma filtering surgery). Typically, a sufficient amount of HI-HEALON is slowly introduced into the vitreous cavity via a syringe fitted with a 27 gauge cannula. By directing the injection, HI-HEALON can be used to separate membranes (e.g., epiretinal membranes) away from the retina for safe excision and release of traction. HEALON also serves to maneuver tissues into the desired position, e.g., to gently push back a detached retina or unroll a retinal flap, and aids in holding the retina against the sclera for reattachment.

### Example 19

### Method for Management of Osteoarthritis with an HI-Loaded Hyaluronic Acid Implant

A hyaluronic acid containing composition containing a hyaluronidase inhibitor is delivered intra-articularly for the symptomatic management (reduction of pain, stiffness, swelling) of osteoarthritis. The presence of the HI controls the rate of degradation of the hyaluronic acid and prolongs the composition's activity in vivo beyond that seen with HA alone (e.g., consistently longer than 6 months in many patients and longer than 1 year in some patients).

An HI-loaded intra-articular hyaluronic acid is prepared by combining a hyaluronidase inhibitor with hyaluronic acid. A variety of HA intra-articular products can be combined with a hyaluronidase inhibitor. Numerous commercially available HA-containing materials are suitable for combining with an HI including: SYNVISC; ORTHOVISC; DUROLANE; HYALGAN (from Fidia/Sanofi-Synthelabo); and HPS and SUPARTZ. It should be noted that some HA products (notably HYVISC by Boehringer Ingelheim Vetmedica, St. Joseph, Mo.) are used in veterinary applications (typically in horses to treat osteoarthritis and lameness).

Although any of the above HA products could be potentially used, in the following example SYNVISC is combined with a hyaluronidase inhibitor in the following manner:

1. A 2.25 ml glass syringe containing 2 ml of hyaluronic acid (SYNVISC; Hylan G-F 20 from Genzyme Biosurgery, or other sources of HA such as DUROLANE) is prepared to contain a hyaluronidase inhibitor as follows:
a. Utilizing aurothiomalate as the hyaluronidase inhibitor, the agent is incorporated into a sustained release delivery system (such as the polymeric microspheres described in Example 12) such that one achieves a concentration of 5 mg/ml of aurothiomalate in the hyaluronic acid (i.e., a total of 10 mg of aurothiomalate contained in microspheres are incorporated into 2 ml of SYNVISC). It should be noted that a range of about 0.2 mg to about 100 mg of aurothiomalate would be of clinical benefit, but about 10 mg is the preferred dosage.
b. Utilizing indomethacin as the hyaluronidase inhibitor, the agent is incorporated into a sustained release delivery system (such as the polymeric microspheres described in Examples 6, 7 and 8) such that one achieves a concentration of 1 mg/ml of indomethacin in the hyaluronic acid (i.e., a total of 2 mg of indomethacin contained in microspheres are incorporated into 2 ml of SYNVISC). It should be noted that a range of about 0.2 mg to about 20 mg of indomethacin would be of clinical benefit, but about 2 mg is the preferred dosage.
c. Utilizing propylene glycol as the hyaluronidase inhibitor, the agent is incorporated into a sustained release delivery system (such as the polymeric microspheres described in Examples 6 to 13) such that one achieves a concentration of 10 mg/ml of propylene glycol in the hyaluronic acid (i.e., a total of 20 mg of propylene glycol contained in microspheres are incorporated into 2 ml of SYNVISC). It should be noted that a range of about 0.5 mg to about 200 mg of propylene glycol would be of clinical benefit, but about 20 mg is the preferred dosage.
d. Utilizing dextran sulphate as the hyaluronidase inhibitor, the agent is incorporated into a sustained release delivery system (such as the polymeric microspheres described in Example 10) such that one achieves a concentration of 10 mg/ml of dextran sulphate in the hyaluronic acid (i.e., a total of 20 mg of dextran sulphate contained in microspheres are incorporated into 2 ml of SYNVISC). It should be noted that a range of about 0.5 mg to about 200 mg of dextran sulphate would be of clinical benefit, but about 20 mg is the preferred dosage.
e. Utilizing fucoidan as the hyaluronidase inhibitor, the agent is incorporated into a sustained release delivery system (such as the polymeric microspheres described in Examples 11 and 13) such that one achieves a concentration of 5 mg/ml of fucoidan in the hyaluronic acid (i.e. a total of 10 mg of fucoidan contained in microspheres are incorporated into 2 ml of SYNVISC). It should be noted that a range of about 0.2 mg to about 100 mg of fucoidan would be of clinical benefit, but about 10 mg is the preferred dosage.
f. Utilizing heparin as the hyaluronidase inhibitor, the agent is incorporated into a sustained release delivery system (such as the polymeric microspheres described in Example 9) such that one achieves a concentration of 1 mg/ml of heparin in the hyaluronic acid (i.e., a total of 2 mg of heparin contained in microspheres are incorporated into 2 ml of SYNVISC). It should be noted that a range of about 0.2 mg to about 100 mg of heparin would be of clinical benefit, but about 2 mg is the preferred dosage.

2. The SYNVISC/microsphere hyaluronidase inhibitor material is sterilized and administered to the patient in the manner described below. Strict aseptic administration technique must be followed during the entire surgical procedure.

A hyaluronic acid containing composition containing a hyaluronidase inhibitor is injected into the joint space for the management of osteoarthritis in a knee joint. The injection site is swabbed with alcohol or other suitable anti-septic solution before injection. The synovial fluid or effusion is removed before injection of the HA-HI implant. The same syringe for removing synovial fluid and for injecting HI-loaded SYNVISC should not be used; however, the same needle should be used. The HA-HI implant is injected using strict aseptic technique into the knee joint through an 18 to 22 gauge needle. To ensure a tight seal and prevent leakage during administration, secure the needle tightly while firmly holding the luer hub. Do not over tighten or apply excessive leverage when attaching the needle or removing the needle guard, as this may break the tip of the syringe. Do not inject anesthetics or any other medications intra-articularly into the knee while administering SYNVISC-HI therapy. This may dilute implant material and affect its safety and effectiveness.

The syringe containing HI-SYNVISC is intended for single use. The contents of the syringe must be used immediately after the syringe has been removed from its packaging. Inject the full 2 ml in one knee only. If treatment is bilateral, a separate syringe must be used for each knee. Discard any unused material. The HA-HI implant is administered by intra-articular injection once a week (one week apart) for a total of three injections for the treatment of painful osteoarthritis of the knee.

It should be apparent to one of skill in the art that other joints (e.g. shoulder, hip, ankle, wrist, etc.) and other species (horses, dogs, cats, etc.) can be administered the material in a similar manner.

### Example 20

### Method for Management of Urinary Incontinence with an HI-Loaded Hyaluronic Acid Bulking Agent

Periurethral and transurethral injections using an HI-loaded HA-bulking agent can be used in the treatment of urinary incontinence. A HI-loaded HA injection can decrease the rate of degradation of the implant and prolong its activity in vivo beyond that seen with HA alone (i.e., consistently greater than 1 year in the majority of patients), such as to sustain the activity of the implant and reduce the need for, and frequency of, subsequent periurethral and transurethral injections.

DEFLUX is a sterile, highly viscous gel of dextranomer microspheres (50 mg/ml) in a carrier gel of non-animal stabilized hyaluronic acid (NASHA, 17 mg/ml), constituting a biocompatible and biodegradable implant. The dextranomer microspheres range in size between 80-250 microns with an average size of about 130 microns. The NASHA acts mainly as a carrier, leaving the dextranomer microspheres at the implant site. DEFLUX (Q-Med/Priority Healthcare) is supplied in a single use disposable sterilized syringe containing 1 ml and is suitable for combining with a hyaluronidase inhibitor in the following manner:

1. A 1 ml syringe of DEFLUX is prepared to contain a hyaluronidase inhibitor as follows:
a. Utilizing aurothiomalate as the hyaluronidase inhibitor, the agent is incorporated into a sustained release delivery system (such as the polymeric microspheres described in Example 12 or incorporated into the dextranomer microspheres) such that one achieves a concentration of 5 mg/ml of aurothiomalate in the hyaluronic acid (i.e., a total of 5 mg of aurothiomalate contained in microspheres are incorporated into 1 ml of DEFLUX). It should be noted that a range of about 0.2 mg to about 100 mg of aurothiomalate would be of clinical benefit, but about 5 mg is the preferred dosage.
b. Utilizing indomethacin as the hyaluronidase inhibitor, the agent is incorporated into a sustained release delivery system (such as the polymeric microspheres described in Examples 6, 7 and 8) such that one achieves a concentration of 1 mg/ml of indomethacin in the hyaluronic acid (i.e., a total of 1 mg of indomethacin contained in microspheres are incorporated into 1 ml of DEFLUX). It should be noted that a range of about 0.2 mg to about 20 mg of indomethacin would be of clinical benefit, but about 1 mg is the preferred dosage.
c. Utilizing propylene glycol as the hyaluronidase inhibitor, the agent is incorporated into a sustained release delivery system (such as the polymeric microspheres described in Examples 6 to 13 or incorporated into the dextranomer microspheres) such that one achieves a concentration of 10 mg/ml of propylene glycol in the hyaluronic acid (i.e., a total of 10 mg of propylene glycol contained in microspheres are incorporated into 1 ml of DEFLUX). It should be noted that a range of about 0.5 mg to about 200 mg of propylene glycol would be of clinical benefit, but about 10 mg is the preferred dosage.
d. Utilizing dextran sulphate as the hyaluronidase inhibitor, the agent is incorporated into a sustained release delivery system (such as the polymeric microspheres described in Example 10 or incorporated into the dextranomer microspheres) such that one achieves a concentration of 10 mg/ml of dextran sulphate in the hyaluronic acid (i.e., a total of 10 mg of dextran sulphate contained in microspheres are incorporated into 1 ml of DEFLUX). It should be noted that a range of about 0.5 mg to about 200 mg of dextran sulphate would be of clinical benefit, but about 10 mg is the preferred dosage.
e. Utilizing fucoidan as the hyaluronidase inhibitor, the agent is incorporated into a sustained release delivery system (such as the polymeric microspheres described in Examples 11 and 13 or incorporated into the dextranomer microspheres) such that one achieves a concentration of 5 mg/ml of fucoidan in the hyaluronic acid (i.e., a total of 5 mg of fucoidan contained in microspheres are incorporated into 1 ml of DEFLUX). It should be noted that a range of about 0.2 mg to about 100 mg of fucoidan would be of clinical benefit, but about 5 mg is the preferred dosage.
f. Utilizing heparin as the hyaluronidase inhibitor, the agent is incorporated into a sustained release delivery system (such as the polymeric microspheres described in Example 9 or incorporated into the dextranomer microspheres) such that one achieves a concentration of 1 mg/ml of heparin in the hyaluronic acid (i.e., a total of 1 mg of heparin contained in microspheres are incorporated into 1 ml of DEFLUX). It should be noted that a range of about 0.2 mg to about 100 mg of heparin would be of clinical benefit, but about 1 mg is the preferred dosage.

2. The DEFLUX/microsphere hyaluronidase inhibitor material is sterilized and administered to the patient in the manner described below.

Transurethral injection of the HI-loaded DEFLUX composition may proceed in the following manner. A single use, pre-loaded syringe with a fine gauge needle (23 gauge transurethral injection needle with a stabilizing cannula) containing 1 ml of the implant material is used. The patient is placed in the lithotomy position and 10 ml of 2% lidocaine is inserted into the urethra for anesthesia. In women, the bladder neck is visualized cystoscopically. Via the injection port of the cystoscope, the needle is inserted at the 4 o'clock position, at a sharp angle, 1-1.5 cm distal to the bladder neck, into the plane just beneath the bladder mucosa. The needle is then advanced with the cystoscope parallel to the long axis of the urethra until it lies just below the mucosa of the bladder neck. The HI-loaded DEFLUX is injected slowly into this site. The procedure is then repeated at the 8 o'clock position. Methylene blue, or other nontoxic coloring agents, can be added to the implant to assist with visualization of the injection.

Periurethral injection of an HI-loaded DEFLUX composition may proceed in the following manner. A single use, pre-loaded syringe with a fine gauge needle (periurethral injection needle) containing 1 ml of the implant material is used. The patient is placed in the lithotomy position, 10 ml of 2% lidocaine is inserted into the urethra for anesthesia, and the bladder neck is visualized cystoscopically (in men the urethra can also be visualized via suprapubic cystoscopic approach). The needle is inserted transvaginally or suprapubically into the area immediately adjacent and lateral to the urethra. When it reaches the appropriate position near the bladder neck (as seen cystoscopically and described above), the HI-loaded HA is injected slowly into this site. Methylene blue, or other nontoxic coloring agents, can be added to the implant to assist with visualization of the injection.

### Example 21

### Method for Management of Fecal Incontinence with an HI-Loaded Hyaluronic Acid Bulking Agent

Fecal incontinence is a common and socially disabling condition that affects up to 11% of North American adults. Incontinence to flatus or feces can be caused by a variety of factors, but is more common in women where the anal sphincter can be damaged during child birth (especially those who have suffered a third degree vaginal tear, required forceps, had large babies, and/or experienced long labor as part of a vaginal delivery). Although the etiology of fecal incontinence is often multifactorial, causes include sphincter injury (obstetric, surgical, accidental), anorectal disease (hemorrhoids, rectal prolapse, inflammatory bowel disease, fistulas, tumors, colon resection, fecal impaction, diarrhea), congenital (spina bifida, meningocele, Hirshsprung's disease), idiopathic, or behavioral (resistance to defecation, dementia, mental retardation). Passive fecal incontinence (i.e., occurring without the patient's awareness) is primarily due to dysfunction of the internal anal sphincter, while urge fecal incontinence (the inability to voluntarily suppress defecation) is usually due to external anal sphincter dysfunction. An HI-loaded HA composition can be injected into the region around the internal or external sphincter to increase sphincter pressure and reduce fecal incontinence. Utilizing a HI-loaded HA injection can sustain the activity of the implant and reduce the need for, and frequency of, peri-anal injections.

DEFLUX is a sterile, highly viscous gel of dextranomer microspheres (50 mg/ml) in a carrier gel of non-animal stabilized hyaluronic acid (NASHA, 17 mg/ml), constituting a biocompatible and biodegradable implant. The dextranomer microspheres range in size between 80-250 microns with an average size of about 130 microns. The NASHA acts mainly as a carrier, leaving the dextranomer microspheres at the implant site. DEFLUX is supplied in a single use disposable sterilized syringe containing 1 ml and is suitable for combining with a hyaluronidase inhibitor in the following manner:

1. A 1 ml syringe of DEFLUX is prepared to contain a hyaluronidase inhibitor as follows:
a. Utilizing aurothiomalate as the hyaluronidase inhibitor, the agent is incorporated into a sustained release delivery system (such as the polymeric microspheres described in Example 12 or incorporated into the dextranomer microspheres) such that one achieves a concentration of 5 mg/ml of aurothiomalate in the hyaluronic acid (i.e., a total of 5 mg of aurothiomalate contained in microspheres are incorporated into 1 ml of DEFLUX). It should be noted that a range of about 0.2 mg to about 100 mg of aurothiomalate would be of clinical benefit, but about 5 mg is the preferred dosage.
b. Utilizing indomethacin as the hyaluronidase inhibitor, the agent is incorporated into a sustained release delivery system (such as the polymeric microspheres described in Examples 6, 7 and 8) such that one achieves a concentration of 1 mg/ml of indomethacin in the hyaluronic acid (i.e., a total of 1 mg of indomethacin contained in microspheres are incorporated into 1 ml of DEFLUX). It should be noted that a range of about 0.2 mg to about 20 mg of indomethacin would be of clinical benefit, but about 1 mg is the preferred dosage.
c. Utilizing propylene glycol as the hyaluronidase inhibitor, the agent is incorporated into a sustained release delivery system (such as the polymeric microspheres described in Examples 6 to 13 or incorporated into the dextranomer microspheres) such that one achieves a concentration of 10 mg/ml of propylene glycol in the hyaluronic acid (i.e., a total of 10 mg of propylene glycol contained in microspheres are incorporated into 1 ml of DEFLUX). It should be noted that a range of about 0.5 mg to about 200 mg of propylene glycol would be of clinical benefit, but about 10 mg is the preferred dosage.
d. Utilizing dextran sulphate as the hyaluronidase inhibitor, the agent is incorporated into a sustained release delivery system (such as the polymeric microspheres described in Example 10 or incorporated into the dextranomer microspheres) such that one achieves a concentration of 10 mg/ml of dextran sulphate in the hyaluronic acid (i.e., a total of 10 mg of dextran sulphate contained in microspheres are incorporated into 1 ml of DEFLUX). It should be noted that a range of about 0.5 mg to about 200 mg of dextran sulphate would be of clinical benefit, but about 10 mg is the preferred dosage.
e. Utilizing fucoidan as the hyaluronidase inhibitor, the agent is incorporated into a sustained release delivery system (such as the polymeric microspheres described in Examples 11 and 13 or incorporated into the dextranomer microspheres) such that one achieves a concentration of 5 mg/ml of fucoidan in the hyaluronic acid (i.e., a total of 5 mg of fucoidan contained in microspheres are incorporated into 1 ml of DEFLUX). It should be noted that a range of about 0.2 mg to about 100 mg of fucoidan would be of clinical benefit, but about 5 mg is the preferred dosage.
f. Utilizing heparin as the hyaluronidase inhibitor, the agent is incorporated into a sustained release delivery system (such as the polymeric microspheres described in Example 9 or incorporated into the dextranomer microspheres) such that one achieves a concentration of 1 mg/ml of heparin in the hyaluronic acid (i.e., a total of 1 mg of heparin contained in microspheres are incorporated into 1 ml of DEFLUX). It should be noted that a range of about 0.2 mg to about 100 mg of heparin would be of clinical benefit, but about 1 mg is the preferred dosage.

2. The DEFLUX/Microsphere Hyaluronidase Inhibitor material is sterilized and administered to the patient in the manner described below.

The HI-DEFLUX implant is administered via direct injection under endoscopic vision in the following manner. A single use, pre-loaded syringe with a fine gauge needle containing 1 ml of the implant material is used. Approximately 10 ml of 2% lidocaine is inserted into the perineal skin or the rectal mucosa depending upon the region of injection selected. The needle is inserted through the skin or the rectal mucosa into the submucosal plane surrounding the anal sphincter. When needle reaches the appropriate position, the HI-loaded HA is injected slowly into the site (typically in 3 injections placed circumferentially, trans-sphincterally, entering away from the anal margin and injecting at, or just above, the dentate line) until symmetry is achieved around the anal canal. Methylene blue, or other nontoxic coloring agents, can be added to the implant to assist with visualization of the injection.

### Example 22

### Hyaluronic Acid Degradation: GPC Molecular Weight Assay

A gel permeation chromatography (GPC) assay (GPC Molecular Weight Assay) was used to determine the effect of various compounds on the degradation of hyaluronic acid over time (as measured by a decrease in the molecular weight of hyaluronic acid upon cleavage by hyaluronidase).

Method:

The GPC system used in the analysis was a BREEZE computerized GPCinstrument (Waters Corporation, Milford, Mass.) equipped with refractive index detection and tandem ULTRAHYDROGEL 1000 and ULTRAHYDROGEL 2000 (Waters Corporation) columns. Water was used as the mobile phase at a flow rate of 1 ml per minute. The injection volume was 50 μl, and the run time was 25 minutes.

A linear calibration curve of of retention time as a function of log molecular weight was prepared using polysaccharide standards ranging from 11,000 to 2 million Daltons (Polymer Laboratories; Church Stretton, UK).

Hyaluronic acid having a molecular weight of between 2 million and 3.5 million daltons (sodium hyaluronate, Lifecore, Chaska Minn.) was diluted to a concentration of 0.5% w/v in water. Hyaluronidase (Sigma Chemical Co. St Louis, Mo.) was diluted in water at a concentration of 1000 units per ml and added in the appropriate volume to achieve the desired final concentration. Hyaluronidase was used at 100 units per ml over a 15 hour incubation time in all experiments. At a concentration of 100 units per ml, the enzyme reduced the molecular weight from 2.5 million to less than 100,000 daltons within 5 hours. HA at a concentration of 0.1% w/v in water (with and without 100 units/ml enzyme) was used as a control sample in each experiment.

Results:

The following compounds were tested to determine their effect on degradation of hyaluronic acid by hyaluronidase: heparin (sodium salt), aurothiomalate, carboxymethylcellulose, dextransulphate, fucoidan. Inhibitors were either added directly to the sample solutions or in small volumes of concentrated solutions. Each of the five compounds tested inhibited degradation of hyaluronic acid by hyaluronidase.

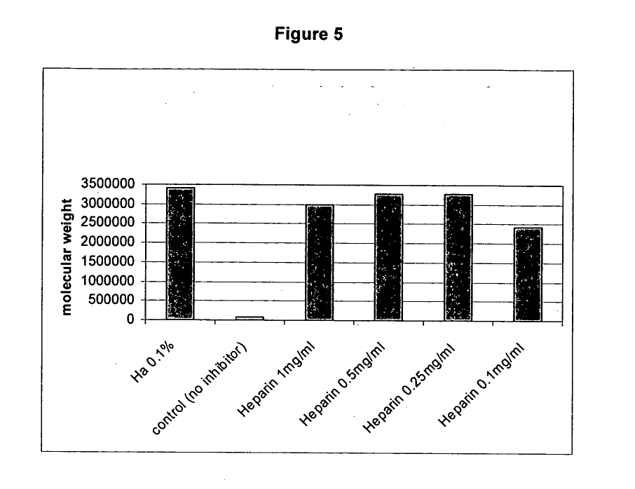

Heparin (sodium salt, Sigma Chemical Co.) was tested at concentrations of 1 mg/ml, 0.5 mg/ml, 0.25 mg/ml and 0.1 mg/ml. Heparin inhibited hyaluronic acid degradation at each of the concentrations tested. Even at concentrations as low as 0.1 mg/ml, the molecular weight of HA decreased from approximately 3.4 million to approximately 2.4 million (Table 5, FIG. 5), representing approximately 70% inhibition of degradation.

TABLE 5

| Sample A | MW (% of HA value) |
|---|---|
| HA control | 100 |
| HA/enzyme control | 2.5 |
| heparin 1 mg/ml | 87 |
| heparin 0.5 mg/ml | 96 |
| heparin 0.25 mg/ml | 96 |
| heparin 0.1 mg/ml | 71 |

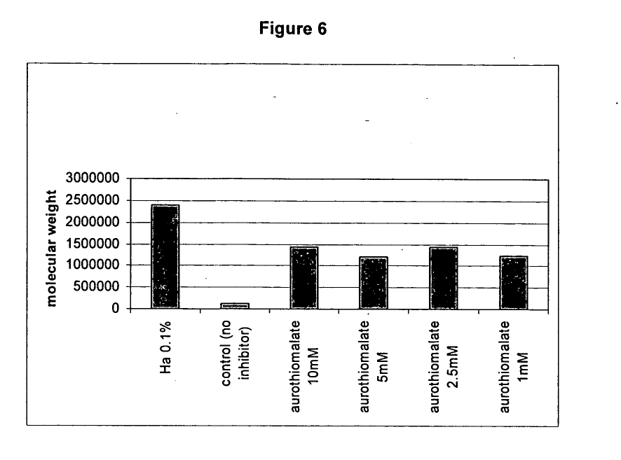

Aurothiomalate (sodium salt, Sigma Chemical Co.) inhibited degradation of HA by more than 50% at concentrations of 10 mM, 5 mM, 2.5 mM, and 1 mM (Table 6, FIG. 6)

TABLE 6

| Sample B | MW (% of HA value) |
|---|---|
| HA control | 100 |
| HA/enzyme control | 5 |
| aurothiomalate 10 mM | 62 |
| aurothiomalate 5 mM | 51 |
| aurothiomalate 2.5 mM | 59 |
| aurothiomalate 1 mM | 51 |

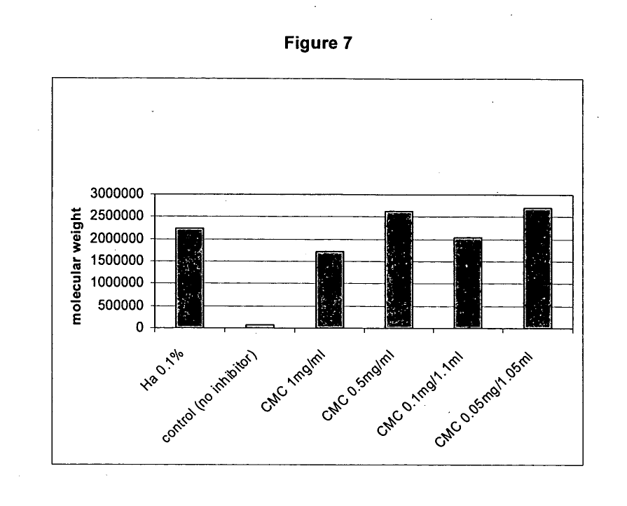

Carboxymethylcellulose (Fisher Scientific) inhibited degradation by hyaluroimidase at concentrations in the range of 0.05 to 1 mg/ml. The molecular weight of the hyaluronic acid after incubation remained close to the original value of 2.25 million (Table 7, FIG. 7).

TABLE 7

| Sample C | MW (% of HA value) |
|---|---|
| HA control | 100 |
| HA/enzyme control | 3 |
| CMC 1 mg/ml | 71 |
| CMC 0.5 mg/ml | 100 |
| CMC 0.1 mg/1.1 ml | 92 |
| CMC 0.05 mg/1.05 ml | 100 |

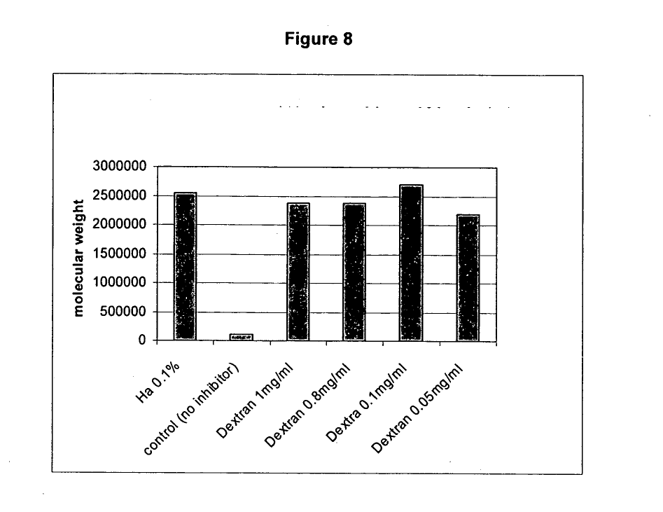

Dextran sulphate (Sigma Chemical Co.) inhibited degradation of hyaluronic acid over a concentration range of 0.05 to 1 mg/ml (Table 8, FIG. 8).

TABLE 8

| Sample D | MW (% of HA value) |
|---|---|
| HA control | 25 |
| HA/enzyme control | 4 |
| dextran 1 mg/ml | 94 |
| dextran 0.8 mg/ml | 93 |
| dextran 0.1 mg/ml | 100 |
| dextran 0.05 mg/ml | 86 |

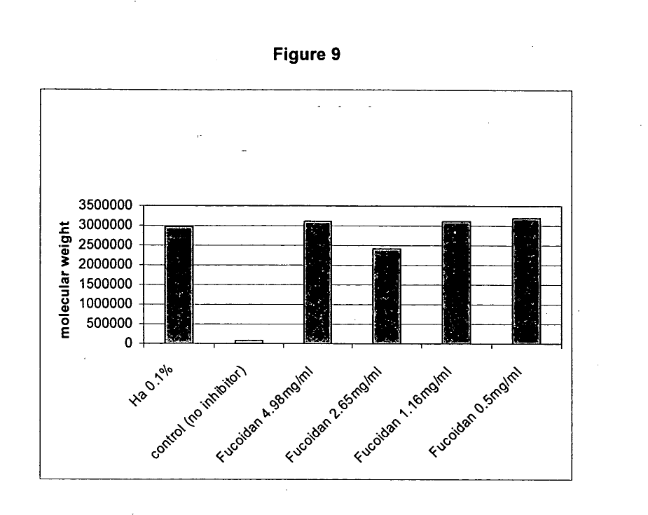

Fucoidan (Sigma Chemical Co.) inhibited degradation of hyaluronic acid over a concentration range of 0.5 to about 5 mg/ml (Table 9, FIG. 9).

TABLE 9

| Sample E | MW (% of HA value) |
|---|---|
| HA control | 100 |
| HA/enzyme control | 2 |
| fucoidan 4.98 mg/ml | 100 |
| fucoidan 2.65 mg/ml | 83 |
| fucoidan 1.16 mg/ml | 100 |
| fucoidan 0.5 mg/ml | 100 |

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A composition comprising hyaluronic acid, a gold compound and a polymer, wherein the gold compound inhibits degradation of hyaluronic acid.

2. The composition of claim 1 wherein the compound inhibits enzyme-induced degradation of hyaluronic acid.

3. The composition of claim 2 wherein the enzyme is hyaluronidase.

4. The composition of claim 1 wherein the gold compound is an organo-gold compound.

5. The composition of claim 1 wherein the gold compound is aurothiomalate or an analogue or derivative thereof.

6. A composition comprising hyaluronic acid, a sulphate-containing polysaccharide and a polymer, wherein the sulphate-containing polysaccharide inhibits degradation of hyaluronic acid.

7. The composition of claim 6 wherein the sulphate-containing polysaccharide inhibits enzyme-induced degradation of hyaluronic acid.

8. The composition of claim 7 wherein the enzyme is hyaluronidase.

9. The composition of claim 6 wherein the sulphate-containing polysaccharide is a fucan.

10. The composition of claim 9 wherein the fucan is fucoidan or an analogue or derivative thereof.

11. The composition of claim 6 wherein the sulphate-containing polysaccharide is dextran sulphate or an analogue or derivative thereof.

12. The composition of claim 6 wherein the sulphate-containing polysaccharide is heparin or an analogue or derivative thereof.

13-15. (canceled)

16. A composition comprising hyaluronic acid and a polymer, wherein the polymer inhibits degradation of hyaluronic acid.

17. The composition of claim 16 wherein the polymer inhibits enzyme-induced degradation of hyaluronic acid.

18. The composition of claim 17 wherein the enzyme is hyaluronidase.

19. The composition of claim 16 wherein the polymer comprises lactic acid residues having the structure (—O—CH(CH₃)—CO—).

20. The composition of claim 16 wherein the polymer comprises ethylene oxide residues having the structure (—OCH₂CH₂—).

21. The composition of claim 16 wherein the polymer comprises poly(lactic acid)-co-poly(ethylene glycol) (PLA-PEG).

22. The composition of claim 16 wherein the polymer comprise poly(L-lactic acid)-co-methoxypoly(ethylene glycol) (MePEG-PLLA) (60:40).

23. The composition of claim 16 wherein the polymer comprises poly(lactic-co-glycolic acid)-co-poly(ethylene glycol) (PLGA-PEG).

24. The composition of claim 16 wherein the polymer comprises poly(caprolactone)-co-poly(ethylene glycol) (PCL-PEG).

25. The composition of claim 16 wherein the polymer is a blend of polymers.

26. The composition of claim 16 wherein the polymer is a blend of poly(lactic acid)-co-poly(ethylene glycol) (PLA-PEG) and poly(L-lactic acid)-co-methoxypoly(ethylene glycol) (MePEG-PLLA).

27. The composition of claim 16, further comprising a gold compound.

28-70. (canceled)

71. A method for reducing pain associated with post-surgical scarring, comprising infiltrating an area surrounding a nerve during a surgical procedure with the composition of any one of claims 1, 6 and 16.

72. A method for preventing surgical adhesions, comprising delivering to a patient in need thereof at a desired location the composition of any one of claims 1 to 48 claims 16 and 16.

73. A method for the repair or augmentation of skin or tissue, comprising injecting into the skin or tissue of a patient in need thereof the composition of any one of claims 1, 6 and 16.

74. The method of claim 73 wherein the injection is into the lips.

75. The method of claim 73 wherein the injection is into the skin on the face.

76. A method of maintaining volume in eye fluid during ocular surgery, comprising delivering to the inside of an eye during an ocular surgery the composition of any one of claims 1, 6 and 16.

77. The method of claim 76 wherein the ocular surgery is cataract extraction surgery, intraocular lens implantation, retinal reattachment, phacoemulsification surgery, corneal transplantation or glaucoma filtering surgery.

78. A method of reducing pain associated with osteoarthritis, comprising injecting into a joint of a patient in need thereof the composition of any one of claims 1, 6 and 16.

79. A method of treating gastroesophageal reflux disease comprising injecting the composition of any one of claims 1, 6 and 16 into the vicinity of the lower esophageal sphincter of a patient.

80. A method for treating or preventing urinary incontinence, comprising administering to a patient in need thereof the composition of any one of claims 1, 6 and 16, such that the urinary incontinence is treated or prevented.

81. The method of claim 80 wherein the composition is administered periurethrally.

82. The method of claim 80 wherein the composition is administered transurethrally.

83. A method of treating or preventing fecal incontinence comprising injecting the composition of any one of claims 1, 6 and 16 into the vicinity of the anal sphincter of a patient, such that the fecal incontinence is treated or prevented.

84-129. (canceled)
